SEQUENCE LISTING (127 sequences)

SEQ ID NO: 1 - DNA, 29 bp, Synthetic construct
  source 1..29: mol_type = other DNA
aattctaatacgactcactatagggtcca

SEQ ID NO: 2 - RNA, 39 nt, Synthetic construct
  source 1..39: mol_type = other RNA
ggggatttagactaccccaaaaacgaaggggactaaaac

SEQ ID NO: 3 - DNA, 28 bp, Synthetic construct
  source 1..28: mol_type = other DNA
ctttcagcggggaggaagggagtaaagt

SEQ ID NO: 4 - DNA, 28 bp, Synthetic construct
  source 1..28: mol_type = other DNA
ttgcgtctggtgtgaaagcttactgctt

SEQ ID NO: 5 - DNA, 28 bp, Synthetic construct
  source 1..28: mol_type = other DNA
ggcgtaaagggagcgcaggcggtcagga

SEQ ID NO: 6 - DNA, 28 bp, Synthetic construct
  source 1..28: mol_type = other DNA
agtggcgaaagcggctctctggtctgtc

SEQ ID NO: 7 - DNA, 28 bp, Synthetic construct
  source 1..28: mol_type = other DNA
ggcagcttgccatactgcgactgacact

SEQ ID NO: 8 - DNA, 28 bp, Synthetic construct
  source 1..28: mol_type = other DNA
ctttcagttgggaagaaaaaaatgacgg

SEQ ID NO: 9 - DNA, 28 bp, Synthetic construct
  source 1..28: mol_type = other DNA
atgggagtttgttgcaccagaagtagct

SEQ ID NO: 10 - DNA, 28 bp, Synthetic construct
  source 1..28: mol_type = other DNA
gagatggattggtgccttcgggaactgt

SEQ ID NO: 11 - DNA, 28 bp, Synthetic construct
  source 1..28: mol_type = other DNA
cagggctacacacgtgctacaatggcat

SEQ ID NO: 12 - DNA, 28 bp, Synthetic construct
  source 1..28: mol_type = other DNA
tagggagctttgagacaggtgctgcatg

SEQ ID NO: 13 - DNA, 28 bp, Synthetic construct
  source 1..28: mol_type = other DNA
ggatgtactgacgctgatgtgcgaaagc

SEQ ID NO: 14 - RNA, 66 nt, Synthetic construct
  source 1..66: mol_type = other RNA
ggggatttagactaccccaaaaacgaaggggactaaaacactttactcccttcctccccgctgaaa

SEQ ID NO: 15 - RNA, 67 nt, Synthetic construct
  source 1..67: mol_type = other RNA
ggggatttagactaccccaaaaacgaaggggactaaaacaagcagtaagctttcacaccagacgcaa

SEQ ID NO: 16 - RNA, 67 nt, Synthetic construct
  source 1..67: mol_type = other RNA
ggggatttagactaccccaaaaacgaaggggactaaaactcctgaccgcctgcgctccctttacgcc

SEQ ID NO: 17 - RNA, 67 nt, Synthetic construct
  source 1..67: mol_type = other RNA
ggggatttagactaccccaaaaacgaaggggactaaaacgacagaccagagagccgctttcgccact

SEQ ID NO: 18 - RNA, 67 nt, Synthetic construct
  source 1..67: mol_type = other RNA
ggggatttagactaccccaaaaacgaaggggactaaaacagtgtcagtcgcagtatggcaagctgcc

SEQ ID NO: 19 - RNA, 67 nt, Synthetic construct
  source 1..67: mol_type = other RNA
ggggatttagactaccccaaaaacgaaggggactaaaacccgtcatttttttcttcccaactgaaag

SEQ ID NO: 20 - RNA, 67 nt, Synthetic construct
  source 1..67: mol_type = other RNA
ggggatttagactaccccaaaaacgaaggggactaaaacagctacttctggtgcaacaaactcccat

SEQ ID NO: 21 - RNA, 67 nt, Synthetic construct
  source 1..67: mol_type = other RNA
ggggatttagactaccccaaaaacgaaggggactaaaacacagttcccgaaggcaccaatccatctc

SEQ ID NO: 22 - RNA, 67 nt, Synthetic construct
  source 1..67: mol_type = other RNA
ggggatttagactaccccaaaaacgaaggggactaaaacatgccattgtagcacgtgtgtagccctg

SEQ ID NO: 23 - RNA, 67 nt, Synthetic construct
  source 1..67: mol_type = other RNA
ggggatttagactaccccaaaaacgaaggggactaaaaccatgcagcacctgtctcaaagctcccta

SEQ ID NO: 24 - RNA, 67 nt, Synthetic construct
  source 1..67: mol_type = other RNA
ggggatttagactaccccaaaaacgaaggggactaaaacgctttcgcacatcagcgtcagtacatcc

SEQ ID NO: 25 - RNA, 28 nt, Synthetic construct
  source 1..28: mol_type = other RNA
actttactcccttcctccccgctgaaag

SEQ ID NO: 26 - RNA, 28 nt, Synthetic construct
  source 1..28: mol_type = other RNA
aagcagtaagctttcacaccagacgcaa

SEQ ID NO: 27 - RNA, 28 nt, Synthetic construct
  source 1..28: mol_type = other RNA
tcctgaccgcctgcgctccctttacgcc

SEQ ID NO: 28 - RNA, 28 nt, Synthetic construct
  source 1..28: mol_type = other RNA
gacagaccagagagccgctttcgccact

SEQ ID NO: 29 - RNA, 28 nt, Synthetic construct
  source 1..28: mol_type = other RNA
agtgtcagtcgcagtatggcaagctgcc

SEQ ID NO: 30 - RNA, 28 nt, Synthetic construct
  source 1..28: mol_type = other RNA
ccgtcatttttttcttcccaactgaaag

SEQ ID NO: 31 - RNA, 28 nt, Synthetic construct
  source 1..28: mol_type = other RNA
agctacttctggtgcaacaaactcccat

SEQ ID NO: 32 - RNA, 28 nt, Synthetic construct
  source 1..28: mol_type = other RNA
acagttcccgaaggcaccaatccatctc

SEQ ID NO: 33 - RNA, 28 nt, Synthetic construct
  source 1..28: mol_type = other RNA
atgccattgtagcacgtgtgtagccctg

SEQ ID NO: 34 - RNA, 28 nt, Synthetic construct
  source 1..28: mol_type = other RNA
catgcagcacctgtctcaaagctcccta

SEQ ID NO: 35 - RNA, 28 nt, Synthetic construct
  source 1..28: mol_type = other RNA
gctttcgcacatcagcgtcagtacatcc

SEQ ID NO: 36 - DNA, 28 bp, Synthetic construct
  source 1..28: mol_type = other DNA
actttactcccttcctccccgctgaaag

SEQ ID NO: 37 - DNA, 28 bp, Synthetic construct
  source 1..28: mol_type = other DNA
aagcagtaagctttcacaccagacgcaa

SEQ ID NO: 38 - DNA, 28 bp, Synthetic construct
  source 1..28: mol_type = other DNA
tcctgaccgcctgcgctccctttacgcc

SEQ ID NO: 39 - DNA, 28 bp, Synthetic construct
  source 1..28: mol_type = other DNA
gacagaccagagagccgctttcgccact

SEQ ID NO: 40 - DNA, 28 bp, Synthetic construct
  source 1..28: mol_type = other DNA
agtgtcagtcgcagtatggcaagctgcc

SEQ ID NO: 41 - DNA, 28 bp, Synthetic construct
  source 1..28: mol_type = other DNA
ccgtcatttttttcttcccaactgaaag

SEQ ID NO: 42 - DNA, 28 bp, Synthetic construct
  source 1..28: mol_type = other DNA
agctacttctggtgcaacaaactcccat

SEQ ID NO: 43 - DNA, 28 bp, Synthetic construct
  source 1..28: mol_type = other DNA
acagttcccgaaggcaccaatccatctc

SEQ ID NO: 44 - DNA, 28 bp, Synthetic construct
  source 1..28: mol_type = other DNA
atgccattgtagcacgtgtgtagccctg

SEQ ID NO: 45 - DNA, 28 bp, Synthetic construct
  source 1..28: mol_type = other DNA
catgcagcacctgtctcaaagctcccta

SEQ ID NO: 46 - DNA, 28 bp, Synthetic construct
  source 1..28: mol_type = other DNA
gctttcgcacatcagcgtcagtacatcc

SEQ ID NO: 47 - DNA, 52 bp, Synthetic construct
  source 1..52: mol_type = other DNA
aattctaatacgactcactatagggtccagggaatattgcaatgggcgcaag

SEQ ID NO: 48 - DNA, 55 bp, Synthetic construct
  source 1..55: mol_type = other DNA
aattctaatacgactcactatagggtccagttgtccggatttattgggcgtaaag

SEQ ID NO: 49 - DNA, 59 bp, Synthetic construct
  source 1..59: mol_type = other DNA
aattctaatacgactcactatagggtccagtagcttaccagaaagggacggctaactac

SEQ ID NO: 50 - DNA, 58 bp, Synthetic construct
  source 1..58: mol_type = other DNA
aattctaatacgactcactatagggtccagtgaaatgcgtagatatatggaggaacac

SEQ ID NO: 51 - DNA, 58 bp, Synthetic construct
  source 1..58: mol_type = other DNA
aattctaatacgactcactatagggtccagtgaaatgcatagatatcacgaggaactc

SEQ ID NO: 52 - DNA, 59 bp, Synthetic construct
  source 1..59: mol_type = other DNA
aattctaatacgactcactatagggtccagaatattggacaatggaccgagagtctgat

SEQ ID NO: 53 - DNA, 57 bp, Synthetic construct
  source 1..57: mol_type = other DNA
aattctaatacgactcactatagggtccagaatcgctagtaatcgcggatcagaatg

SEQ ID NO: 54 - DNA, 59 bp, Synthetic construct
  source 1..59: mol_type = other DNA
aattctaatacgactcactatagggtccacttacctggtcttgacatccacagaacttt

SEQ ID NO: 55 - DNA, 58 bp, Synthetic construct
  source 1..58: mol_type = other DNA
aattctaatacgactcactatagggtccagtgataaactggaggaaggtggggatgac

SEQ ID NO: 56 - DNA, 59 bp, Synthetic construct
  source 1..59: mol_type = other DNA
aattctaatacgactcactatagggtccacttacctactcttgacatccgaagaagaac

SEQ ID NO: 57 - DNA, 56 bp, Synthetic construct
  source 1..56: mol_type = other DNA
aattctaatacgactcactatagggtccagaaatgcgtagagatgtggaggaatac

SEQ ID NO: 58 - DNA, 27 bp, Synthetic construct
  source 1..27: mol_type = other DNA
cgggtaacgtcaatgagcaaaggtatt

SEQ ID NO: 59 - DNA, 30 bp, Synthetic construct
  source 1..30: mol_type = other DNA
ttacaccgagaattccagtctcccctactg

SEQ ID NO: 60 - DNA, 26 bp, Synthetic construct
  source 1..26: mol_type = other DNA
gttgagccatagccttttactccaga

SEQ ID NO: 61 - DNA, 26 bp, Synthetic construct
  source 1..26: mol_type = other DNA
gactaccagggtatctaatcctgttc

SEQ ID NO: 62 - DNA, 30 bp, Synthetic construct
  source 1..30: mol_type = other DNA
ctagtaatcatcgtttactgcgtggactac

SEQ ID NO: 63 - DNA, 31 bp, Synthetic construct
  source 1..31: mol_type = other DNA
cacgtatttagccgtcacttcttctgttggt

SEQ ID NO: 64 - DNA, 26 bp, Synthetic construct
  source 1..26: mol_type = other DNA
gtggtaaccgccctctttgcagttag

SEQ ID NO: 65 - DNA, 28 bp, Synthetic construct
  source 1..28: mol_type = other DNA
gacttaacccaacatttcacaacacgag

SEQ ID NO: 66 - DNA, 26 bp, Synthetic construct
  source 1..26: mol_type = other DNA
ggactacgacatactttatgaggtcc

SEQ ID NO: 67 - DNA, 28 bp, Synthetic construct
  source 1..28: mol_type = other DNA
gacttaacccaacatttcacaacacgag

SEQ ID NO: 68 - DNA, 25 bp, Synthetic construct
  source 1..25: mol_type = other DNA
atccccaaatcgacaccgtttacag

SEQ ID NO: 69 - DNA, 28 bp, Synthetic construct
  source 1..28: mol_type = other DNA
tcaagtcattttttattcattgtgttat

SEQ ID NO: 70 - DNA, 28 bp, Synthetic construct
  source 1..28: mol_type = other DNA
atattattcttattcttcttttttcttt

SEQ ID NO: 71 - DNA, 28 bp, Synthetic construct
  source 1..28: mol_type = other DNA
tcatattattcttattcttcttttttct

SEQ ID NO: 72 - DNA, 28 bp, Synthetic construct
  source 1..28: mol_type = other DNA
cattacttcattatactagcctcgttat

SEQ ID NO: 73 - DNA, 28 bp, Synthetic construct
  source 1..28: mol_type = other DNA
tgctgtatcttatctcctttgtccactt

SEQ ID NO: 74 - DNA, 28 bp, Synthetic construct
  source 1..28: mol_type = other DNA
ggtctgtttgctatattttcaataattt

SEQ ID NO: 75 - DNA, 28 bp, Synthetic construct
  source 1..28: mol_type = other DNA
cagtgatggcagtgtttatttaaaaaat

SEQ ID NO: 76 - RNA, 67 nt, Synthetic construct
  source 1..67: mol_type = other RNA
ggggatttagactaccccaaaaacgaaggggactaaaacataacacaatgaataaaaaatgacttga

SEQ ID NO: 77 - RNA, 67 nt, Synthetic construct
  source 1..67: mol_type = other RNA
ggggatttagactaccccaaaaacgaaggggactaaaacaaagaaaaaagaagaataagaataatat

SEQ ID NO: 78 - RNA, 67 nt, Synthetic construct
  source 1..67: mol_type = other RNA
ggggatttagactaccccaaaaacgaaggggactaaaacagaaaaaagaagaataagaataatatga

SEQ ID NO: 79 - RNA, 67 nt, Synthetic construct
  source 1..67: mol_type = other RNA
ggggatttagactaccccaaaaacgaaggggactaaaacataacgaggctagtataatgaagtaatg

SEQ ID NO: 80 - RNA, 67 nt, Synthetic construct
  source 1..67: mol_type = other RNA
ggggatttagactaccccaaaaacgaaggggactaaaacaagtggacaaaggagataagatacagca

SEQ ID NO: 81 - RNA, 67 nt, Synthetic construct
  source 1..67: mol_type = other RNA
ggggatttagactaccccaaaaacgaaggggactaaaacaaattattgaaaatatagcaaacagacc

SEQ ID NO: 82 - RNA, 67 nt, Synthetic construct
  source 1..67: mol_type = other RNA
ggggatttagactaccccaaaaacgaaggggactaaaacattttttaaataaacactgccatcactg

SEQ ID NO: 83 - RNA, 28 nt, Synthetic construct
  source 1..28: mol_type = other RNA
ataacacaatgaataaaaaatgacttga

SEQ ID NO: 84 - RNA, 28 nt, Synthetic construct
  source 1..28: mol_type = other RNA
aaagaaaaaagaagaataagaataatat

SEQ ID NO: 85 - RNA, 28 nt, Synthetic construct
  source 1..28: mol_type = other RNA
agaaaaaagaagaataagaataatatga

SEQ ID NO: 86 - RNA, 28 nt, Synthetic construct
  source 1..28: mol_type = other RNA
ataacgaggctagtataatgaagtaatg

SEQ ID NO: 87 - RNA, 28 nt, Synthetic construct
  source 1..28: mol_type = other RNA
aagtggacaaaggagataagatacagca

SEQ ID NO: 88 - RNA, 28 nt, Synthetic construct
  source 1..28: mol_type = other RNA
aaattattgaaaatatagcaaacagacc

SEQ ID NO: 89 - RNA, 28 nt, Synthetic construct
  source 1..28: mol_type = other RNA
attttttaaataaacactgccatcactg

SEQ ID NO: 90 - DNA, 28 bp, Synthetic construct
  source 1..28: mol_type = other DNA
ataacacaatgaataaaaaatgacttga

SEQ ID NO: 91 - DNA, 28 bp, Synthetic construct
  source 1..28: mol_type = other DNA
aaagaaaaaagaagaataagaataatat

SEQ ID NO: 92 - DNA, 28 bp, Synthetic construct
  source 1..28: mol_type = other DNA
agaaaaaagaagaataagaataatatga

SEQ ID NO: 93 - DNA, 28 bp, Synthetic construct
  source 1..28: mol_type = other DNA
ataacgaggctagtataatgaagtaatg

SEQ ID NO: 94 - DNA, 28 bp, Synthetic construct
  source 1..28: mol_type = other DNA
aagtggacaaaggagataagatacagca

SEQ ID NO: 95 - DNA, 28 bp, Synthetic construct
  source 1..28: mol_type = other DNA
aaattattgaaaatatagcaaacagacc

SEQ ID NO: 96 - DNA, 28 bp, Synthetic construct
  source 1..28: mol_type = other DNA
attttttaaataaacactgccatcactg

SEQ ID NO: 97 - DNA, 59 bp, Synthetic construct
  source 1..59: mol_type = other DNA
aattctaatacgactcactatagggtccagtagatgggcatttatctttattatcacga

SEQ ID NO: 98 - DNA, 59 bp, Synthetic construct
  source 1..59: mol_type = other DNA
aattctaatacgactcactatagggtccatcccttattctttcttatttcctctgtttt

SEQ ID NO: 99 - DNA, 59 bp, Synthetic construct
  source 1..59: mol_type = other DNA
aattctaatacgactcactatagggtccattcccttattctttcttatttcctctgttt

SEQ ID NO: 100 - DNA, 59 bp, Synthetic construct
  source 1..59: mol_type = other DNA
aattctaatacgactcactatagggtccaccctttgttaaaaacgatagcagaaaaata

SEQ ID NO: 101 - DNA, 59 bp, Synthetic construct
  source 1..59: mol_type = other DNA
aattctaatacgactcactatagggtccagattttcttatcggatttcacaaatgactg

SEQ ID NO: 102 - DNA, 59 bp, Synthetic construct
  source 1..59: mol_type = other DNA
aattctaatacgactcactatagggtccatacaataatgctagatttatcgcactagtg

SEQ ID NO: 103 - DNA, 59 bp, Synthetic construct
  source 1..59: mol_type = other DNA
aattctaatacgactcactatagggtccagatacgatgatcttcaggcgttatacaatg

SEQ ID NO: 104 - DNA, 59 bp, Synthetic construct
  source 1..59: mol_type = other DNA
aattctaatacgactcactatagggtccagaaacctttgctataatacctatcgtcttt

SEQ ID NO: 105 - DNA, 30 bp, Synthetic construct
  source 1..30: mol_type = other DNA
ggcaagtccaaaaataaaataaggattgaa

SEQ ID NO: 106 - DNA, 30 bp, Synthetic construct
  source 1..30: mol_type = other DNA
atgaagaggaataaggatgaagatgagatg

SEQ ID NO: 107 - DNA, 30 bp, Synthetic construct
  source 1..30: mol_type = other DNA
gaagaggaataaggatgaagatgagatgag

SEQ ID NO: 108 - DNA, 30 bp, Synthetic construct
  source 1..30: mol_type = other DNA
tgcaatatgaataatagaggtatcaggaga

SEQ ID NO: 109 - DNA, 30 bp, Synthetic construct
  source 1..30: mol_type = other DNA
aatcactttttcactcgtataggtcagttg

SEQ ID NO: 110 - DNA, 30 bp, Synthetic construct
  source 1..30: mol_type = other DNA
gcagtacaaatgaaaattgaaccagattta

SEQ ID NO: 111 - DNA, 30 bp, Synthetic construct
  source 1..30: mol_type = other DNA
gattttataaattgatgccatctgctgatt

SEQ ID NO: 112 - DNA, 30 bp, Synthetic construct
  source 1..30: mol_type = other DNA
aagcgacaatctaacattaaagaagtgata

SEQ ID NO: 113 - DNA, 507 bp, Streptococcus mutans
  source 1..507: mol_type = genomic DNA
atgtctatctttaaaaataaattatttgtcattttatttgcctttcttatgttacttgtagatgggcatttatctttattatcacgaatcttatttcaaaatcagtttattgtgtcaagtcattttttattcattgtgttattgttttatactcttgtattcaatccttattttatttttggacttgcctgtgttttaggaattatttatgatttttattatttaggggtatataatttgggaattgcaactatgttgtatcctttaacaattgttattatgtttaaattatggaaacatattccaaatggtcctgttcaacgctttctagttttttttatcctaatcttttttcttgattttgctagtattggtatggcctatttatatcaattgacagcttatcctttaaatgattttatcacttataatttagcaccttccttaatttttaatatactggcttttctcttctttcaaaaacttttagaaaggatttacctatga

SEQ ID NO: 114 - DNA, 549 bp, Scardovia wiggsiae
  source 1..549: mol_type = genomic DNA
atgatagttaaggggcgggaaataatcccgccccttaactatttcccttattctttcttatttcctctgttttcatattattcttattcttcttttttctttttaccctttcgcactcgccctcttcacctgcgttctccttcttccttccctcatctcatcttcatccttattcctcttcatcctcgctccggtcgtcatcccgcttcaccttccaatctcactctgcccttccttacaccctgccttttcttactctccaatctcatctcacctccttatttttcgatctcaccccgttcttccttactctccgatttcatctcacttctttactttccgaccttttatccacatacatatgtagccccacttccgacctcactccatctctccctaccttctgacctcactccataccctccttactttccgatcgtactccaccatctccttactctccaatctcaccccatcccctcacccaactcgaagatacggatcctcctccgacggcctcctcccacatccgtacagctgaaactataa

SEQ ID NO: 115 - DNA, 1023 bp, Aggregatibacter actinomycetemcomitans
  source 1..1023: mol_type = genomic DNA
atgaaagcattactccgaaaaatccgtttagccttaggcaaaatgttgctagacaaaaatgttcaggggcaggctttgccggcaaatccgaaaattattgtgttacaacaagacggaaaaatcggagattatattgtcagttcatttattttcagagaattaaaacgacataaccctaaaatgcaggttgatgttgtatgttcacctaaaaatgtcaatttatttgaacaaaatccatctattgatcattgttttattttaaatagaaaagaacattgcgcttacagcagaatgggaaaacaactttcacatgaacattatgatgtattaattaatttaccggtattattacggaatcgtgatttgtggctaacccgattaattcatgcaaaaaataatatcggctataaaaaacaaaactacaaactatttaatttgaatgttactcaagatcaattacatttttccaaagtttatgcagaagccattaaattatgtggtgtaaaagatattaatcttgaatatgacattccaaatcacagtgataaaaaagaagatattgcaaatttcatacaaaaaaatcatcttgtcgattgcatcgctataaacttttttggtgcagccggcaccagaaaatttacggaacaaaacatttatcggtttatggaaaaatttaaagccgaaaataaaaaagcgctgttactaacttaccccgaagtaacccctttgttaaaaacgatagcagaaaaatataccaatgcttttatctatgaaaatactgaaaatatctttgatacgataacattacttcattatactagcctcgttatttctcctgatacctctattattcatattgcagcaggattaaataaaaaaattattggattttataagctcgctgataaagaaaactttacgcattggaatccaaactgtaaaaataaaacatatatccttaattttattgaaaatgtcaatgaaatctctcccgatgaaattaaatcagaatggttaaaataa

SEQ ID NO: 116 - DNA, 972 bp, Porphyromonas gingivalis
  source 1..972: mol_type = genomic DNA
gtggcaaaatccgatagaacgtttttaatgccggttacgatattgatcgccctcgtgtgtaggatgctgtcctacgcatgggggctttctgctgatatttctcttcaagagtgtagtcttcccctcttcggcacgatccctttcttatggcaaaccgttatctcgtttgccattggcttggttgcatcttttattgcggttcgcttcagtgctttctatcttctgcttcatgaaggtggattccggccatttgctttcttgatgattcttttgctgaacacccatcaggttttcttccccatgcagccatactcgctgtcgatactgctgctgatagtactctttttctgcctttttggcacttatggccgtaataatatcccgcccaagatgcttaacgtaggcttttgtgtcgggctttcggctgtgttgtggtctccttcgctgcttcttgcaccgtttgtgctcatacagttttatctgatgaagagtttgtctttcaaaaacttgattgcatttttcttcggactgttgcttcctctgtggtgctgtcttcctttattggtattggccggacaggagcagtttgttatcgacaatatgacccttttgactcggtggggattttcttatcggatttcacaaatgactgcgtggaaggggctgtatccggctctactgtcagtgctgtatcttatctcctttgtccacttgcaactgacctatacgagtgaaaaagtgattgcacgcatttacttcttcagccttttgtgtgccggttgttatatgctgctactctctgccgtgatgcctgctgcttccgaaggattcgtatatctggctaccttccctgtgtcggtacatgcagctcgtttcttgaattctctgaatcgtcgtgttgccaatatcttgattccttttacgtttttggttttcttgtcctcttttttgctgtcttcttttttcctttag

SEQ ID NO: 117 - DNA, 504 bp, Acinetobacter baumannii
  source 1..504: mol_type = genomic DNA
atggctaaacgctacttacccttctacaataatgctagatttatcgcactagtgttagtcggtctgtttgctatattttcaataatttttaaatatttagagttaaatattacaataaatctggttcaattttcatttgtactgcttctccctttaagtcaaatttatttggcttataaaggtatgctcgatgcattaaagcttgatggtttaaatcagtcagaacgagataggttgacttcaactgtggacataagaagtaagtcatctttatatgtggctatgctttttattattcttgtttttagtatgtatatacttaatttattaggcttactttcagctaagcatcttttagctctaatactttctgttggactcacctcaatttttagcttcttcttagcttggtctgacttaagagaaatctctttgcttgaaaaaacattaaaagatcgcaaagaatcaagagaggcaaaagcaaaagtattgagcaataagtaa

SEQ ID NO: 118 - DNA, 1167 bp, Klebsiella pneumoniae
  source 1..1167: mol_type = genomic DNA
gtgaaacagacccattatttcaccgtgaattttaccggcttcaccactgccgcctcggaagagcaaagctatttacgcctcattgcaggcgagcatgccttttataccgataaacgccattttaaagacccctcgctttttgaccgtctgaggctcggccagccgctacatatcggcacttgccgcctaaaggatggcagttactggatccactggttaagcgatggccacattttgctcgaaccttcccggcagccgctgagtattaaaaagcttcttcgacctgtcggcttggtcttttttgtttgcttgatcattctcgcttgtgctccatggttggatatttttttattattatgttttatcacaatgttggtgaatcccttcggcatcttctctttttgctcttgcaatttctcactcaggttacgtgttctcaatgcgaaaatgcagcgaacgaagcaaggcgatatttcattctgtcagcgtctggaaattctacccgctacttctgaaaggcatccaccatcggccaacaacgagatagcgctgccaaaagaatttgcgcttgaagaaggcgtgattctgaatagttattatcaaatatggatcgctacgagggtaaggccttattttatcaaagaaggcgcctgttttcagctggcgactcgtcactcgtttttttcagcgcgcttgacctggctgagcaatgccctgaataatcgttgtcgcccgccatttcttgccgatggggactgggttatcgccgcacgccggcatggatacgatgatcttcaggcgttatacaatgtcagtgatggcagtgtttatttaaaaaatagcccctttcatcccggcaatcagcagatggcatcaatttataaaatcgcgtacggcctcgtactcatagtgatgttttttacgctcaccatcaaactgcacgaccccatgcttaatctgggcagttggccattttatcgtgaaatgcttaccaggctgacaggcaccctgctcccgattaacctgctgttaatagtgatggagtttgtccgtatgattgcccatcccctgtcacgtcgggtggccggtagagtgaaggtacatcaggccatattacgttatgccagacgtcagggcgccagaatgacgccatgggaactgaagtaa

SEQ ID NO: 119 - DNA, 708 bp, Staphylococcus aureus
  source 1..708: mol_type = genomic DNA
ttggtagtattgttatcttatagttgcagttgtatctttagttttctatatcaatttatatctatagaagaaacgtcatttgattatttacatagaagatcgaaatgtgattattgtaattcatcactcaaatggtatgaattaatgccgattattagttttttattattaaaagggcgatgtcgaaactgtcgaaagcgtatttccctaacacatttcttaggggaaacctttgctataatacctatcgtctttattaagtatgatttcacatacgtaaatgctacgctatttataactacatatgtttttctgcttatttttactatgaccgatatcacttctttaatgttagattgtcgcttaattataatttattgtatcgtttctctctcgttaagtatgatttatccagtagcttttatcattattagtatgaccacgcatatattctactttttatttcgggcatatattggttatggtgacgttttactaatatctgcactttctttgtttttccctctccaattcactatttatgtcattttatttacatttgtcattgctggtttagttgctttaattaccatgatatttaagccgattaaactattaccccttgttccatttatatttatttcattttttatcaattcacttttttataatgatatccatcaatttttaggaggcgtatatttttga

SEQ ID NO: 120 - DNA, 28 bp, Synthetic construct
  source 1..28: mol_type = other DNA
aactacatatgtttttctgcttattttt

SEQ ID NO: 121 - RNA, 67 nt, Synthetic construct
  source 1..67: mol_type = other RNA
ggggatttagactaccccaaaaacgaaggggactaaaacaaaaataagcagaaaaacatatgtagtt

SEQ ID NO: 122 - RNA, 28 nt, Synthetic construct
  source 1..28: mol_type = other RNA
aaaaataagcagaaaaacatatgtagtt

SEQ ID NO: 123 - DNA, 28 bp, Synthetic construct
  source 1..28: mol_type = other DNA
aaaaataagcagaaaaacatatgtagtt

SEQ ID NO: 124 - DNA, 22 bp, Synthetic construct
  source 1..22: mol_type = other DNA
tatagtgagtcgtattaatttc

SEQ ID NO: 125 - DNA, 3457 bp, Leptotrichia wadei
  source 1..3457: mol_type = genomic DNA
atgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacgatatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagctggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaat

SEQ ID NO: 126 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 127 - DNA, 25 bp, Synthetic construct
  source 1..25: mol_type = other DNA
gaaattaatacgactcactataggg

(19) United States
(12) Patent Application Publication
(10) Pub. No.: US 2025/0305070 A1
(43) Pub. Date: Oct. 2, 2025

(54) METHODS AND SYSTEMS FOR DETECTION OF ORAL BACTERIA

(71) Applicant: ADA FORSYTH INSTITUTE, INC., Cambridge, MA (US)

(72) Inventors: Wenyuan Shi, Cambridge, MA (US); Batbileg Bor, Cambridge, MA (US); Jet Liu, Cambridge, MA (US); Camden Carmichael, Cambridge, MA (US)

(73) Assignee: ADA FORSYTH INSTITUTE, INC., Cambridge, MA (US)

(21) Appl. No.: 19/074,178

(22) Filed: Mar. 7, 2025

Related U.S. Application Data
(63) Continuation of application No. PCT/US2023/073807, filed on Sep. 8, 2023.
(60) Provisional application No. 63/375,214, filed on Sep. 9, 2022.
(60) Provisional application No. 63/384,920, filed on Nov. 23, 2022.

(51) Int. Cl. C12Q 1/689; C12N 9/22; C12N 15/11

(52) U.S. Cl. CPC C12Q 1/689; C12N 9/226; C12N 15/11; C12N 2310/20; C12N 2320/10; C12Q 2600/158

(57) ABSTRACT

Provided are methods and systems for detection of oral bacteria.

### PRIORITY CLAIM

This application is a continuation of International Patent Application No. PCT/US2023/073807, filed Sep. 8, 2023, which claims the benefit of U.S. Provisional Patent Application Nos. 63/375,214, filed Sep. 9, 2022, and 63/384,920, filed Nov. 23, 2022, all of which are incorporated herein by reference in their entirety.

### SEQUENCE LISTING

This application contains an ST.26 compliant Sequence Listing, which is submitted concurrently in xml format via EFS-Web or Patent Center and is hereby incorporated by reference in its entirety. The .xml copy, created on Sep. 8, 2023 is named 145231-8001WO00 Sequence Listing.xml and is 124,984 bytes in size.

### BACKGROUND

Both globally and in the United States, two main oral diseases account for a large part of the oral disease load and cause a tremendous amount of economic and medical burden: dental caries and periodontal (gum) disease. These diseases have an etiology in microbial pathogenesis and are known to be correlated with well-associated microorganisms. Microorganisms within the oral cavity are also known to include pathogens that can exacerbate systemic diseases in other body sites. Despite the overwhelming evidence that the human oral microbiome contains harmful pathogens, the ability to reliably detect and diagnose the presence of these oral microorganisms via saliva is limited. Furthermore, a majority of the current diagnostic tests available for oral microorganisms are qPCR or Next Generation Sequencing (NGS) based. These two methods, however, require complex time-consuming procedures, specialized equipment, and technical analysis. Thus, there is a clear need for a rapid, low-cost detection option for oral bacteria that is suitable in point-of-care situations.

### SUMMARY

The present technology relates to methods and systems for detection of oral bacteria. Rapid, accessible, and accurate diagnostics are recognized as critical to early disease intervention and precision treatment. Yet in the dental field, where decades of research have linked oral microbes to numerous diseases, detection of oral bacterial pathogens is largely limited to high-cost, complex, and time-intensive methods. Widely employed oral bacterial detection techniques such as qPCR and sequencing often require shipment to a central facility, incur high costs, and remain unsuitable for broad patient profiling. This is despite decades of evidence that specific species or strains of oral bacteria may be the likely keystone agents of oral diseases and are associated with an increasing number of systemic diseases. Specific oral bacteria such as F. nucleatum, S. mutans, and P. gingivalis have been implicated in systemic diseases including various cancers, digestive diseases, cardiovascular diseases, and neurodegenerative diseases. In order to facilitate precision oral medicine that can effectively treat conditions associated with highly pathogenic species or strains of oral bacteria, there is a clear need for the field of dentistry to move towards novel diagnostics suitable for widespread use.

Here, the inventors have tailored the CRISPR-Cas based assay SHERLOCK into a rapid detection platform for oral bacteria that can be easily adaptable to clinical diagnostics. As described herein, the inventors have developed a computational pipeline to generate constructs suitable with SHERLOCK and experimentally validated detection using the constructs for seven prominent oral bacterial pathogens. As shown in the examples, with SHERLOCK, the constructs detected their target nucleic acid sequence within the single-molecule range and remained specific in the presence of off-target oral bacterial and human DNA. Further, the assay was adapted to detect target bacteria from unprocessed saliva samples. This platform is broadly scalable for the detection of oral microorganisms and marks an expansion of molecular diagnostics into the field of oral health.

In some aspects, provided are CRISPR-RNAs (crRNAs) comprising a spacer that binds a target nucleic acid sequence in a gene in an oral bacterium. In certain embodiments, the gene is a species-specific gene. In certain embodiments, the oral bacterium may be from a species selected from the group consisting of Aggregatibacter actinomycetemcomitans, Acinetobacter baumannii, Klebsiella pneumoniae, Porphyromonas gingivalis, Staphylococcus aureus, Streptococcus mutans, and Scardovia wiggsiae. In certain embodiments, the species-specific gene may comprise a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus). In certain embodiments, the spacer may comprise a sequence that is the same as, or differs no more than 1, 2, 3, 4, or 5 nucleotides from, a sequence selected from the group consisting of the spacer RNA sequences set forth in SEQ ID NOs:83-89, and 122. In certain embodiments, the crRNA comprises a sequence that is the same as, or differs no more than 1, 2, 3, 4, or 5 nucleotides from, a sequence selected from the group consisting of the crRNA sequences set forth in SEQ ID NOs:76-82, and 121. In certain embodiments, the target nucleic acid comprises a sequence that is the same as, or differs no more than 1, 2, 3, 4, or 5 nucleotides from, a sequence selected from the group consisting of the protospacer sequences set forth in SEQ ID NOs:69-75, and 120. In certain embodiments, the species-specific gene may be from an oral bacterium from a genera or species set forth below in the Detailed Description section under the heading “Oral Bacteria.” In certain embodiments, the crRNA may further comprise a crRNA backbone sequence. In certain embodiments, the crRNA backbone sequence may comprise a sequence that is the same as, or differs no more than 1, 2, 3, 4, or 5 nucleotides from, a sequence set forth in SEQ ID NO:2.

In certain aspects, provided herein are nucleic acids comprising one or more sequences encoding one or more crRNAs as described herein. In certain embodiments, the nucleic acid comprises a sequence encoding a crRNA comprising a spacer that binds a target nucleic acid sequence in a gene in an oral bacterium. In certain embodiments, the gene is a species-specific gene. In certain embodiments, the oral bacterium may be from a species selected from the group consisting of Aggregatibacter actinomycetemcomitans, Acinetobacter baumannii, Klebsiella pneumoniae, Porphyromonas gingivalis, Staphylococcus aureus, Streptococcus mutans, and Scardovia wiggsiae. In certain embodiments, the species-specific gene may comprise a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus). In certain embodiments, the spacer may comprise a sequence that is the same as, or differs no more than 1, 2, 3, 4, or 5 nucleotides from, a sequence selected from the group consisting of the spacer RNA sequences set forth in SEQ ID NOs:83-89, and 122. In certain embodiments, the crRNA comprises a sequence that is the same as, or differs no more than 1, 2, 3, 4, or 5 nucleotides from, a sequence selected from the group consisting of the crRNA sequences set forth in SEQ ID NOs:76-82, and 121. In certain embodiments, the target nucleic acid comprises a sequence that is the same as, or differs no more than 1, 2, 3, 4, or 5 nucleotides from, a sequence selected from the group consisting of the protospacer sequences set forth in SEQ ID NOs:69-75, and 120. In certain embodiments, the species-specific gene may be from an oral bacterium from a genera or species set forth below in the Detailed Description section under the heading “Oral Bacteria.” In certain embodiments, the crRNA may further comprise a crRNA backbone sequence. In certain embodiments, the crRNA backbone sequence may comprise a sequence that is the same as, or differs no more than 1, 2, 3, 4, or 5 nucleotides from, a sequence set forth in SEQ ID NO:2. In certain embodiments, the nucleic acid further comprises a sequence encoding an RNA-guided nuclease described herein, for example, a Cas13a protein. In certain embodiments, the Cas13a protein may be any one of the Cas13a proteins disclosed herein. In certain embodiments, nucleic acid further comprises one or more sequences encoding a third, fourth, fifth, sixth, seventh, eighth, ninth, or tenth crRNA.

In certain aspects, provided herein are compositions comprising one or more crRNAs as described herein. In certain embodiments, the composition further comprises an RNA-guided nuclease described herein (e.g., Cas13a protein). In certain embodiments, the compositions may comprise any of the nucleic acids described herein, for example, the nucleic acid comprising a sequence encoding a crRNA as described herein, a sequence encoding an RNA-guided nuclease molecule described herein, or a combination thereof. In certain embodiments, the compositions further comprise a third, fourth, fifth, sixth, seventh, eighth, ninth, or tenth crRNA.

In certain aspects, provided herein are CRISPR-nuclease detection systems comprising (i) an RNA-guided nuclease; and (ii) a first crRNA as described herein. In certain embodiments, the first crRNA comprises a spacer that binds a target nucleic acid sequence in a gene in an oral bacterium. In certain embodiments, the gene is a species-specific gene. In certain embodiments, the oral bacterium may be from a species selected from the group consisting of Aggregatibacter actinomycetemcomitans, Acinetobacter baumannii, Klebsiella pneumoniae, Porphyromonas gingivalis, Staphylococcus aureus, Streptococcus mutans, and Scardovia wiggsiae. In certain embodiments, the species-specific gene may comprise a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus). In certain embodiments, the spacer may comprise a sequence that is the same as, or differs no more than 1, 2, 3, 4, or 5 nucleotides from, a sequence selected from the group consisting of the spacer RNA sequences set forth in SEQ ID NOs:83-89, and 122. In certain embodiments, the crRNA comprises a sequence that is the same as, or differs no more than 1, 2, 3, 4, or 5 nucleotides from, a sequence selected from the group consisting of the crRNA sequences set forth in SEQ ID NOs:76-82, and 121. In certain embodiments, the target nucleic acid comprises a sequence that is the same as, or differs no more than 1, 2, 3, 4, or 5 nucleotides from, a sequence selected from the group consisting of the protospacer sequences set forth in SEQ ID NOs:69-75, and 120. In certain embodiments, the species-specific gene may be from an oral bacterium from a genera or species set forth below in the Detailed Description section under the heading “Oral Bacteria.” In certain embodiments, the first crRNA may further comprise a crRNA backbone sequence. In certain embodiments, the first crRNA backbone sequence may comprise a sequence that is the same as, or differs no more than 1, 2, 3, 4, or 5 nucleotides from, a sequence set forth in SEQ ID NO:2. In certain embodiments, the CRISPR-nuclease detection system further comprises a second, third, fourth, fifth, sixth, seventh, eighth, ninth, tenth, etc. crRNA of any crRNA described herein. In certain embodiments, the RNA-guided nuclease may be a Cas13 protein. In certain embodiments, the RNA-guided nuclease may be a Cas13a protein. In certain embodiments, the Cas13a protein may be encoded by a nucleic acid sequence set forth in SEQ ID NO:24. In certain embodiments, the Cas13a protein may comprise the amino acid sequence set forth in SEQ ID NO:25. In certain embodiments, the CRISPR-nuclease detection system may further comprise one or more components selected from the group consisting of a forward primer, a reverse primer, a reporter, replication protein A (RPA), RNAse inhibitor, ribonucleoside tri-phosphate (rNTP) mix, and T7 RNA Polymerase. In certain embodiments, the forward primer may comprise a sequence that binds the gene 5′ of the spacer sequence. In certain embodiments, the forward primer may comprise a sequence that is the same as, or differs no more than 1, 2, 3, 4, or 5 nucleotides from, a sequence set forth in Table 5 or Table 7. In certain embodiments, the reverse primer may comprise a sequence that binds the gene 3′ of the spacer sequence. In certain embodiments, the reverse primer comprises a sequence that is the same as, or differs no more than 1, 2, 3, 4, or 5 nucleotides from, a sequence set forth in Table 5 or Table 7. In certain embodiments, the reporter may comprise a molecule that releases a signal upon cleavage of the reporter by the RNA-guided nuclease. In certain embodiments, the detection system may be a specific high-sensitivity enzymatic reporter unlocking (SHERLOCK) detection platform.

In certain aspects, provided herein are methods of detecting one or more oral bacteria in a biological sample. In certain embodiments, the methods of detecting may comprise (a) contacting the biological sample with a CRISPR nuclease detection system as set forth herein and (b) detecting the presence of the one or more oral bacteria in the biological sample. In certain aspects, provided herein are methods for treating and/or preventing an oral disease caused by an oral bacterium in a subject in need thereof. In certain embodiments, the method of treating and/or preventing may comprise (a) contacting a biological sample from the subject with the CRISPR-nuclease detection system as set forth herein; (b) detecting the presence of the one or more bacteria in the biological sample; and (c) treating and/or preventing the disease. In certain embodiments, the CRISPR-nuclease detection system of the methods may further comprise one or more components selected from the group consisting of a replication protein A (RPA), RNAse inhibitor, and ribonucleoside tri-phosphate (rNTP) mix. In certain embodiments, the forward primer comprises a sequence that binds the gene 5′ of the spacer sequence. In certain embodiments, the forward primer may comprise a sequence that is the same as, or differs no more than 1, 2, 3, 4, or 5 nucleotides from, a sequence set forth in Table 5 or Table 7. In certain embodiments, the reverse primer may comprise a sequence that binds the gene 3′ of the spacer sequence. In certain embodiments, the reverse primer may comprise a sequence that is the same as, or differs no more than 1, 2, 3, 4, or 5 nucleotides from, a sequence set forth in Table 5 or Table 7. In certain embodiments, the reporter may comprise a molecule that releases a signal upon cleavage of the reporter by the RNA-guided nuclease. In certain embodiments, the biological sample may be saliva. In certain embodiments, ethylene glycol-bis(β-aminoethyl ether)-N,N,N′,N′-tetraacetic acid (EGTA) and dithiothreitol (DTT) are added to the biological sample prior to step (a) of contacting the biological sample with the CRISPR-nuclease detection system. In certain embodiments, EGTA, DTT, and the biological sample are heated at about 90° C. to about 120° C., for example, about 95° C., for about 15 minutes prior to step (a). In certain embodiments, EGTA may be added at a concentration of about 100 mM, 200 mM, 300 mM, 400 mM, 500 mM, 600 mM, 700 mM, 800 mM, about 900 mM, or about 1000 mM. In certain embodiments, DTT may be added at a concentration of about 0.5 mM, 1 mM, 5 mM, or 10 mM. In certain embodiments, the oral disease may be one or more selected from the group comprising a gum disease (e.g., periodontitis, gingivitis), systemic disease, halitosis, cavities, sensitive and loose teeth, cancer, strep throat, oro-dental trauma, noma, ulcers, sores, acute necrotizing ulcerative gingivitis, root caries, and a combination thereof. In certain embodiments, detecting the presence of the one or more oral bacteria in the biological sample occurs upon release of the signal.

In certain aspects, provided herein are kits comprising any one of the CRISPR-nuclease detection systems disclosed herein. In certain embodiments, the kit may further comprise EGTA and DTT. In certain embodiments, the kit may comprise a laminar flow strip. In certain embodiments, the components of the kit may be lyophilized on the laminar flow strip. In certain embodiments, the kit may be a rapid-detection system.

In certain aspects, provided herein are methods of lysing bacteria. In certain embodiments, the methods of lysing bacteria may comprise contacting a biological sample comprising bacteria with ethylene glycol-bis(β-aminoethyl ether)-N,N,N′,N′-tetraacetic acid (EGTA) and dithiothreitol (DTT); and heating the biological sample at a temperature for a period of time. In certain embodiments, the period of time may be about 15 minutes. In certain embodiments, the temperature is about 90° C. to about 120°. In certain embodiments the temperature is about 95° C. In certain embodiments, EGTA may be at a concentration of about 100 mM, 200 mM, 300 mM, 400 mM, 500 mM, 600 mM, 700 mM, 800 mM, about 900 mM, or about 1000 mM. In certain embodiments, DTT may be at a concentration of about 0.5 mM, 1 mM, 5 mM, or 10 mM.

### BRIEF DESCRIPTION OF THE DRAWINGS

FIGS. 1A-1C show design and testing of CRISPR-RNAs (crRNAs) targeting the DNA of the 16S ribosomal RNA (16S rRNA) gene of different bacteria. FIG. 1A shows a workflow diagram of the computation pipeline that generates species-specific crRNA and primers targeting bacterial 16S rRNA gene to be used with SHERLOCK. FIG. 1B is a schematic displaying the protocol for cross-sensitivity assays against other oral pathogens. FIG. 1C is a heat map displaying cross-reactivities of designed 16S rRNA gene targeting crRNAs against other pathogens. Normalized signal at 35 minutes of detection is displayed. White boxes indicate untested pairs. Target genomic DNA (gDNA) was added at a concentration of 1 picomolar (pM) per reaction. E. coli (Ec), S. mutans (Sm), P. gingivalis (Pg), K. pneumoniae (Kp), A. actinomycetemcomitans (Aa), A. baumannii (Ab), S. wiggsiae (Sw), and F. nucleatum (Fn).

FIGS. 2A-2B show a computational pipeline to generate crRNAs and primer pairs targeting conserved species-specific genes for oral pathogens. FIG. 2A shows a workflow diagram of the computation pipeline that generates specifies-specific crRNA and primers for SHERLOCK. FIG. 2B is a schematic illustrating the placement and architecture of crRNA and primer design for SHERLOCK. FIG. 2C is a schematic showing synthesis of crRNA.

FIGS. 3A-3I show the validation of species-specific designed crRNA and primer pairs for detection of seven established oral pathogens from saliva. FIG. 3A shows detection of target gDNA of seven oral pathogens using each synthesized crRNA. Dotted line indicates the 35-minute mark where most of the signals reach saturation. Target gDNA was added at a concentration of 1 picomolar (pM) per reaction (600,000 copies/rxn). FIG. 3B shows a representative example of the specificity of SHERLOCK detection using designed crRNAs against other common oral pathogen targets. This specific example displays K. pneumoniae crRNA detection against other common oral pathogens. S. mutans (Sm), P. gingivalis (Pg), K. pneumoniae (Kp), A. actinomycetemcomitans (Aa), A. baumannii (Ab), and S. wiggsiae (Sw). FIG. 3C is a heat map displaying cross-reactivity of designed crRNAs against all seven pathogens. To generate this heat map, for each crRNA, cross-reactivity assays were performed to generate fluorescent readout traces (as represented in FIG. 3A). The signals from those traces at 35 minutes of detection were then extracted and normalized to 100%. Target gDNA was added at a concentration of 1 pM per reaction. S. mutans (Sm), P. gingivalis (Pg), K. pneumoniae (Kp), A. actinomycetemcomitans (Aa), A. baumannii (Ab), and S. wiggsiae (Sw) were targeted. For FIG. 3C, all individual plots are shown in FIGS. 3D-3I.

FIGS. 3D-3I shows fluorescence trace plots displaying the specificity of the Pg (FIG. 3D), Kp (FIG. 3E), Sw (FIG. 3F), Sa (FIG. 3G), Ab (FIG. 3H), Aa (FIG. 3I) crRNA-primer sets against other bacterial targets.

FIGS. 4A-4I show specificity of species-specific crRNAs and detection in saliva crRNA background. FIG. 4A is a schematic displaying the steps for specificity assays against background saliva gDNA in addition to what is shown in FIGS. 3A-3I. FIG. 4B shows a representative readout of specificity testing against background saliva gDNA. This specific example displays K. pneumoniae crRNA detection against background saliva gDNA. FIG. 4C is a heat map displaying the specificity of designed crRNAs against isolated saliva gDNA. To generate this heat map, for each crRNA, cross-reactivity assays with background saliva gDNA were performed to generate fluorescent readout traces (as represented in FIG. 4B). The signals from those traces at 35 minutes of detection were then extracted and normalized to 100%, 10 ng of background saliva gDNA was added per reaction. Target gDNA was added at a concentration of 1 picomolar (pM) per reaction. S1, S2, and S3 indicate saliva samples collected from three different healthy volunteers.

FIGS. 4D-4I shows individual fluorescence trace plot of FIG. 4C as the specific pathogens Pg (FIG. 40), Kp (FIG. 4E), Sw (FIG. 4F), Sa (FIG. 4G), Ab (FIG. 4H), Aa (FIG. 4I) crRNA-primer set against background saliva gDNA.

FIGS. 5A-5E show sensitivity of species-specific crRNAs and detection. FIG. 5A shows sensitivity of species-specific Scardovia wiggsiae (Sw) crRNA. Sw crRNA was tested against isolated gDNA from a patient saliva sample spiked with Sw gDNA at varying concentrations (10 ng background concentration). FIG. 5B shows fluorescent signal at 30 minutes against varying concentrations of Sw gDNA. Detection was measured against 10 ng of background patient saliva gDNA. FIG. 5C further shows SHERLOCK detection sensitivity at 35 minutes of decreasing 10-fold concentrations of Sm gDNA alone or combined with 10 ng of saliva gDNA. In addition to FIG. 5B, a control experiment without background gDNA was tested to show the difference between sensitivity. FIG. 5D shows individual traces of SHERLOCK detection from FIG. 5C at 35 minutes of decreasing 10-fold concentrations of Sm gDNA alone. FIG. 5E shows individual traces of SHERLOCK detection from FIG. 5C at 35 minutes of decreasing 10-fold concentrations of Sm gDNA alone combined with 10 ng of saliva gDNA.

FIGS. 6A-6K show oral pathogen detection directly from saliva samples. FIG. 6A is a schematic of the steps of the SHERLOCK assay when testing directly on saliva samples. To process the saliva bacteria for the SHERLOCK, two steps were incorporated: addition of ethylene glycol-bis(β-aminoethyl ether)-N,N,N′,N′-tetraacetic acid (EGTA) and dithiothreitol (DTT), as well as treatment of the samples at 95° C. for 15 min. This process is referred to herein as SHERLOCK-EGTA+DTT. FIG. 6B shows the readout when using SHERLOCK-EGTA+DTT to detect pathogens directly from saliva sample at varying 95° C. heating times. K. pneumoniae (Kp) crRNA was used to detect artificially spiked 300 Kp colony forming units (CFUs)/reaction in saliva. FIG. 6C shows a representative readout using SHERLOCK to detect oral pathogens directly from saliva samples. This specific example displays A. actinomycetemcomitans (Aa) specific detection directly from saliva samples. 6 μL of saliva collected from healthy volunteers were spiked with 300 Aa CFUs then added to each experimental reaction. 6 μL of unspiked saliva collected from the same volunteer as the experimental trial were used as a negative control. FIG. 6C shows direct testing on saliva background of four of the seven oral pathogens. Saliva was added at a volume of 6 microliters per reaction. Saliva was spiked at a concentration of 300 CFUs/reaction of indicated bacteria. “Saliva” and “Saliva and Bacteria” indicate saliva samples and saliva samples spiked with target bacteria, respectively. Normalized fluorescent signal at 35 minutes is displayed. A. actinomycetemcomitans (Aa), A. baumannii (Ab), K. pneumoniae (Kp), S. mutans (Sm). FIG. 6D shows specificity and fluorescent signal of all seven crRNA-primer sets targeting all seven pathogens when used with SHERLOCK-EGTA+DTT and unprocessed saliva samples. Saliva was spiked at a concentration of 300 indicated target bacterial cells per reaction. Fluorescent signal at 35 minutes is displayed. Each point represents a technical replicate. All individual plots from FIG. 6D are shown in FIGS. 6E-6K. Specificity and fluorescent signal of crRNA-primer sets when used with SHERLOCK-EGTA+DTT and unprocessed saliva samples spiked at a concentration of 300 Pg (FIG. 6E), Kp (FIG. 6F), Sw (FIG. 6G), Sa, (FIG. 6H), Ab (FIG. 6I), Aa (FIG. 6J), Sm (FIG. 6K) bacterial cells per reaction.

FIGS. 7A-7D illustrate direct comparison between fluorescent signal from isolated target bacterial gDNA compared to live bacterial cells that spiked into unprocessed saliva. FIGS. 7A-7B show fluorescent signal at 35 minutes produced by SHERLOCK with Sm (FIG. 7A) or Pg (FIG. 7B) gDNA and SHERLOCK-EGTA+DTT spiked with an equivalent concentration of live Sm or Pg cells. FIGS. 7C-7D shows individual fluorescent traces of FIGS. 7A and 7B produced by SHERLOCK with Sm (FIG. 7C) or Pg (FIG. 70) gDNA and SHERLOCK-EGTA+DTT spiked with an equivalent concentration of live Sm (FIG. 7C) or Pg (FIG. 70) cells.

FIGS. 8A-8G show clinical validation of SHERLOCK-EGTA+DTT detection. FIG. 8A shows 16S rRNA sequencing of taxonomic predictions for 30 clinical saliva samples. This is a well adopted and industry used alternative method to detect bacteria.

FIG. 8B shows individual traces of SHERLOCK-EGTA+DTT detection of 30 healthy subject samples using the Sm crRNA-primer set. 6 μl of 1:4 diluted healthy subject saliva was added per reaction. FIG. 8C shows 16S rRNA sequencing methods compared to SHERLOCK-EGTA+DTT Sm detection. Top bar graph displays 16S rRNA relative abundance (%) of Sm from 16S rRNA sequencing. Lower heatmap displays the min-max normalized signal of Sm SHERLOCK-EGTA+DTT detection at 35 minutes. FIG. 8D shows individual traces of SHERLOCK-EGTA+DTT detection of 30 healthy subject samples using the Pg crRNA-primer set. 6 μl of 1:4 diluted healthy subject saliva were added per reaction. FIG. 8E shows 16S rRNA sequencing compared to SHERLOCK-EGTA+DTT Pg detection. Upper bar graph displays 16S rRNA relative abundance (%) of Pg from 16S rRNA sequencing. Bottom heatmap displays the min-max normalized signal of Pg SHERLOCK-EGTA+DTT detection at 35 minutes. FIG. 8F shows individual traces of SHERLOCK-EGTA+DTT detection of 30 healthy subject samples using the Sw crRNA-primer set. 6 μl of 1:4 diluted healthy subject saliva were added per reaction. FIG. 8G shows 16S rRNA sequencing compared to SHERLOCK-EGTA+DTT Sw detection. Upper bar graph displays 16S rRNA relative abundance (%) of Sw from 16S rRNA sequencing. Bottom heatmap displays the min-max normalized signal of Sw SHERLOCK-EGTA+DTT detection at 35 minutes.

### DETAILED DESCRIPTION

Next-generation sequencing (NGS) and novel imaging techniques have revealed complex ways in which opportunistic microbial pathogens play a role in oral diseases. Given the high cost and extended time of NGS, however, point-of-care patient testing and large-scale profiling of the microbiome remains impractical.

In certain aspects, the present technology addresses this need by engineering the newly pioneered specific high-sensitivity enzymatic reporter unlocking (SHERLOCK) detection platform into a rapid detection system capable of identifying oral bacteria without the need for complex equipment or sequencing. The SHERLOCK system specifically uses Clustered Regularly Interspaced Short Palindromic Repeats (CRISPR) associated protein 13 (Cas13) combined with recombinase polymerase amplification (RPA) to detect DNA within the femtomolar range.

In certain aspects, the present technology tailors the SHERLOCK platform to detect oral bacteria directly from a biological sample (e.g., saliva sample). In certain embodiments, primer pairs and crRNAs compatible with SHERLOCK have been designed that target conserved species-specific genes of oral bacteria. Unlike crRNAs that target 16S rRNA genes, which often incur non-specific signal, the specifically designed crRNAs surprisingly display a much higher propensity for the specificity required of a diagnostic. As demonstrated in the working examples, the detection of seven oral bacterial pathogens directly from saliva samples were experimentally validated using specifically designed constructs for the bacteria. With rapid, easy-to-use detection of oral bacteria directly from saliva samples, a potential paradigm shift from standard oral care is possible where dentists are capable of detecting oral bacteria with ease at routine appointments. With patient oral microbiome profiles in hand, one could imagine dentists empowered to prescribe individualized care capable of treating each patient's unique abundance of bacteria.

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value, such as an amount or concentration and the like, is meant to encompass variations of 20%, 10%, 5%, 1%, 0.5%, or even 0.1% of the specified amount.

The term “subject” refers to a mammalian subject, preferably a human. A “subject in need thereof” may refer to a subject who has been diagnosed with a disease, or is at an elevated risk of developing a disease. The phrases “subject” and “patient” are used interchangeably herein.

### Computational Pipeline

As provided herein, to detect oral bacteria using the SHERLOCK system, the inventors designed a computational pipeline that can generate species-specific primers and guide RNAs for any known oral bacteria by targeting genes. In certain embodiments, the genes may be species-specific genes. As shown in the examples below, detection for seven oral pathogens that have established etiologies for either caries, periodontitis, or systemic disease was experimentally validated. The detection remained consistent and specific even in the presence of off-target human and oral microbial DNA. Further, detection was achieved directly from saliva samples. These results, achievable within an hour, demonstrate the clinical utility of this platform and open the door to future applications at routine dental appointments.

Given a bacterial species, the computational pipeline described herein extracted all MetaPhIAn 4.0 marker genes attributed to that particular species (Blanco-Miguez 2022). The pipeline divided each marker gene into every possible 28 bp protospacer. For each possible protospacer, the pipeline searched for ancillary primers compatible with RPA using Primer3 (Untergasser 2012) with the previously described thresholds of (Kellner 2019): primer length between 25-35 bp; Tm between 54-67° C.; and GC % between 20-80%. To the 5′ end of each left primer, a T7 polymerase promoter sequence was added (5′-AATTCTAATACGACTCACTATAGGGTCCA-3′) (SEQ ID NO:1) (Kellner 2019). crRNA sequences were then generated from each protospacer by adding a crRNA backbone sequence (5′-GGGGAUUUAGACUACCCCAAAAACGAAGGGGACUAAAAC-3′) (SEQ ID NO:2) (Gootenberg 2017) to the 5′ of the reverse complement of the protospacer.

For each candidate crRNA and its corresponding primer pair (crRNA-primer set), the putative RNA amplicon produced by RPA and T7 transcription was predicted. The minimum free energy (MFE) of the RNA amplicon secondary structure was computed using ViennaRNA v2.5.1 (Lorenz 2011). As a higher secondary structure MFE of the crRNA target has been suggested to increase crRNA-protospacer binding (Kellner 2019; Gootenberg 2017), crRNA-primer sets were sorted in decreasing RPA amplicon secondary structure MFE. For the synthesis of crRNAs, a ssDNA template sequence was generated by adding a T7 promoter sequence to the 3′ end of the reverse complement of the crRNA sequence (5′-TATAGTGAGTCGTATTAATTTC-3′) (SEQ ID NO:124).

To test the efficacy of the computational pipeline to produce viable constructs, synthesized seven crRNA-primer sets were synthesized. Each set was designed to target one of seven oral pathogens (Table 4). All primers and crRNA-ssDNA templates were ordered from IDT. crRNAs were synthesized using the HiScribe T7 kit (NEB, #E2050S).

In certain embodiments, crRNAs and corresponding primer pairs may be designed for any target using the computational pipeline described herein. In certain embodiments, crRNAs may be designed using the computational pipeline to bind a target nucleic acid in a species-specific gene.

### CRISPR-Nuclease Detection Systems

In certain aspects, provided herein are CRISPR-nuclease detection systems. In certain embodiments, the detection system may be the SHERLOCK system, which is described in Kellner 2019, which is incorporated by reference herein. Detection of nucleic acids through the SHERLOCK system consists of four key steps: (1) amplification of a target region; (2) Cas13 guide/CRISPR RNA (crRNA) recognition of the target region; (3) cleavage of reporter RNAs by Cas13; and (4) fluorescence readout of cleaved reporter RNAs. The first step, amplification of a target region, utilizes the isothermal amplification technique, recombinase polymerase amplification (RPA), to generate many copies of a target region. Next, a crRNA bound to Cas13a in a complex recognizes an about 28 bp region on the target amplicon, also called a protospacer. Upon crRNA binding to the protospacer, Cas13a exhibits indiscriminate RNAse activity, cleaving nearby RNAs. SHERLOCK leverages the collateral cleavage activity of Cas13a by including within the reaction reporter RNAs that contain fluorophores. While the fluorophores of the reporter RNAs are typically quenched upon cleavage of the reporter RNAs by Cas13a, the fluorophores emit a fluorescent signal. This fluorescent signal can be quantified over time and equated to detection. In combining Cas13a detection with RPA, SHERLOCK is capable of detecting targets in the single molecule range in a rapid fashion without the need for complex equipment.

In certain embodiments, CRISPR-nuclease detection system may comprise (i) an RNA-guided nuclease; and (ii) a first crRNA comprising a spacer that binds a target nucleic acid sequence in a gene in an oral bacterium. In certain embodiments, the crRNA may comprise a crRNA as set forth herein. In certain embodiments, the CRISPR-nuclease detection system may comprise any one or more of the components listed in Table 3. In certain embodiments, the one or more components may be selected from the group consisting of a forward primer, a reverse primer, a reporter, replication protein A (RPA), RNAse inhibitor, ribonucleoside tri-phosphate (rNTP) mix, and T7 RNA Polymerase.

The forward primer may be any of the forward primers described herein, such as those found in Table 5 or Table 7. In certain embodiments, the forward primer may comprise a sequence that binds the gene 5′ of the spacer sequence. The reverse primer may be any of the reverse primers described herein, such as those found in Table 5 or Table 7. In certain embodiments, the reverse primer may comprise a sequence that binds the gene 3′ of the spacer sequence.

The reporter may comprise a molecule that releases a signal upon cleavage of the reporter by the RNA-guided nuclease.

The CRISPR-nuclease detection system disclosed herein may further comprise a second, third, fourth, fifth, sixth, seventh, eighth, ninth, or tenth crRNA.

### CRISPR-RNAs

In some aspects, provided herein are CRISPR-RNAs (crRNAs) (also referred to herein as guide RNAs (gRNAs)). In certain embodiments, the crRNA comprises a spacer (complementary region) that binds to a target nucleic acid sequence. In certain embodiments, the target nucleic acid sequence may be in a gene in an oral bacterium. In certain embodiments, the gene may be a species-specific gene.

A “species-specific gene” as used herein refers to a gene or a set of genes that are unique to a particular species of bacteria and are not found in other species. The species-specific gene is an element within the genome of a given biological species that lacks a homologous counterpart in the genomes of other distinct species. In certain embodiments, a species of bacteria may comprise one or more species-specific genes.

In certain embodiments, the target nucleic acid sequence may comprise a protospacer. In certain embodiments, the crRNA may bind the reverse complement of the protospacer.

The length of the spacer is generally between 15 and 30 nucleotides in length. In certain embodiments, the spacer may be 15, 16, 17, 18, 29, 20, 21, 22, 23, 24, 25, 26, 27, 28, 29, or 30 nucleotides in length. In certain embodiments, the spacer may be 28 nucleotides in length. In certain embodiments, the spacer may be fully complementary to the target nucleic acid sequence. In certain embodiments, the spacer may be partially complementary to the target nucleic acid sequence (e.g., at least 80%, 85%, 90%, 95%, 98%, or 99% complementary).

In certain embodiments, the spacer may comprise a sequence that is the same as, or differs no more than 1, 2, 3, 4, or 5 nucleotides from, a sequence selected from the group consisting of the spacer RNA sequences set forth in SEQ ID NOs:83-89, and 122.

In certain embodiments, the crRNA may comprise a sequence that is the same as, or differs no more than 1, 2, 3, 4, or 5 nucleotides from, a sequence selected from the group consisting of the crRNA sequences set forth in SEQ ID NOs:76-82, and 121.

In certain embodiments, the species-specific gene comprises a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus). In certain embodiments, the target nucleic acid may comprise a sequence that is the same as, or differs no more than 1, 2, 3, 4, or 5 nucleotides from, a sequence selected from the group consisting of the protospacer sequences set forth in SEQ ID NOs:69-75, and 120.

In certain embodiments, the crRNA may comprise a crRNA backbone sequence. In certain embodiments, the crRNA backbone sequence may comprise a sequence that is the same as, or differs no more than 1, 2, 3, 4, or 5 nucleotides from a sequence set forth in SEQ ID NO:2.

### RNA-Guided Nucleases

In certain embodiments, the RNA-guided nuclease used herein may be a Cas13 protein. In certain embodiments, the Cas13 protein may be a Cas13a protein. In certain embodiments, the Cas13a protein may be from the genus Leptotrichia. In certain embodiments, the Cas13a protein may be Leptotrichia wadei Cas13a (LwCas13a) protein. In certain embodiments, the Cas13a protein may be encoded by the nucleotide sequence set forth in SEQ ID NO:125 (i.e., human LwCas13a). In certain embodiments, the Cas13a protein may be encoded by a nucleotide sequence comprising one or more nucleotide substitutions, or comprising a sequence that is at least 80% identical (e.g., at least 80%, at least 85%, at least 90%, at least 95%, at least 96%, at least 97%, at least 98%, at least 99%, or 100% identical), to SEQ ID NO:125. In certain embodiments, the Cas13a protein may comprise the amino acid sequence set forth in SEQ ID NO:126 (i.e., human LwCas13a). In certain embodiments, the Cas13a protein may comprise one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to SEQ ID NO:126.

### Oral Bacteria

In certain embodiments, the crRNA may target a species-specific gene of an oral bacteria. In certain embodiments, the oral bacteria may be from a genus selected from the group consisting of Porphyromonas, Streptococcus, Fusobacterium, Aggregatibacter, Escherichia, Klebsiella, Treponema, Tannerella, Pseudomonas, Acinetobacter, Staphylococcus, Enterococcus, Lactobacilli, Actinomyces, Prevotella, Campylobacter, Saccharibacteria, Selenomonas, Corynebacterium, Leptotrichia, Veillonella, Haemophilius, Solobacterium, Atopobium, Eubacterium, Filifactor, Scardovia, Propionibacterium, Saccharibacteria, Propionivibrio, Pyramidobacter, Rothia, Solobacterium, Bacillus, Bacteroidaceae, Bacteroides, Bifidobacterium, Bacteroidetes, Capnocyophaga, Chloroflexi, Catonella, Colibacter, Dialister, Enterococcus, Fretibacterium, Gemella, Gracilibacteria, Helicobacter, Mogibacterium, Mycoplasma, Neisseria, Olsenella, Oribacterium, Paenibacillus, Parvimonas, Peptostreptococcaceae, Peptoniphilus, Absconditabacteria, and Anaerococcus.

In certain embodiments, the oral bacteria may be from a genus selected from the group consisting of Abiotrophia, Absconditabacteria, Achromobacter, Acidipropionibacterium, Acidovorax, Acinetobacter, Actinomyces, Aeriscardovia, Aerococcus, Afipia, Aggregatibacter, Agrobacterium, Alkalihalobacillus, Alloiococcus, Alloprevotella, Alloscardovia, Anaerococcus, Anaeroglobus, Anaerolineae, Anoxybacillus, Aquamicrobium, Arachnia, Arcanobacterium, Arsenicicoccus, Arthrospira, Atopobium, Bacillus, Bacteroidales, Bacteroides, Bacteroidetes, Bartonella, Bdellovibrio, Bifidobacterium, Bordetella, Bosea, Bradyrhizobium, Brevibacterium, Brevundimonas, Brochothrix, Brucella, Bulleidia, Burkholderia, Butyrivibrio, Campylobacter, Capnocytophaga, Cardiobacterium, Catonella, Caulobacter, Cellulosimicrobium, Centipeda, Chlamydia, Chryseobacterium, Cloacibacterium, Clostridiales, Colibacter, Comamonas, Corynebacterium, Cronobacter, Cryptobacterium, Cupriavidus, Cutibacterium, Delftia, Dermabacter, Desulfobulbus, Desulfomicrobium, Desulfovibrio, Dialister, Dietzia, Dolosigranulum, Eggerthella, Eggerthia, Eikenella, Enhydrobacter, Enterobacter, Enterococcus, Erysipelothrix, Erysipelotrichaceae, Erythrobacter, Escherichia, Eubacterium, Fannyhessea, Fastidiosipila, Filifactor, Finegoldia, Flavitalea, Fretibacterium, Fusobacterium, Gardnerella, Gemella, Gracilibacteria, Granulicatella, Haematobacter, Haemophilus, Helicobacter, Janibacter, Jeotgalicoccus, Johnsonella, Jonquetella, Kingella, Klebsiella, Kluyvera, Kocuria, Kytococcus, Lachnoanaerobaculum, Lachnospiraceae, Lacticaseibacillus, Lactiplantibacillus, Lactobacillus, Lactococcus, Lancefieldella, Lautropia, Lawsonella, Lentilactobacillus, Leptothrix, Leptotrichia, Levilactobacillus, Ligilactobacillus, Limosilactobacillus, Listeria, Lysinibacillus, Megasphaera, Mesorhizobium, Micavibrio, Microbacterium, Micrococcus, Mitsuokella, Mobiluncus, Mogibacterium, Mollicutes, Moraxella, Mycobacterium, Mycolicibacterium, Mycoplasma, Mycoplasmopsis, Neisseria, Neisseriaceae, Novosphingobium, Odoribacter, Olegusella, Olsenella, Oribacterium, Oryzomicrobium, Ottowia, Paenibacillus, Paracoccus, Parascardovia, Parvimonas, Pedobacter, Peptidiphaga, Peptococcus, Peptoniphilaceae, Peptoniphilus, Peptostreptococcaceae, Peptostreptococcus, Phocaeicola, Porphyromonas, Prevotella, Propionibacteriaceae, Proteus, Pseudoleptotrichia, Pseudomonas, Pseudoramibacter, Pyramidobacter, Ralstonia, Rhodobacter, Rhodopseudomonas, Riemerella, Roseomonas, Rothia, Ruminococcaceae, Saccharibacteria, Sanguibacter, Scardovia, Schaalia,Schlegelella,Segetibacter, Selenomonas, Serratia, Shuttleworthia, Simonsiella, Slackia, Sneathia, Solobacterium, Sphingomonas, Staphylococcus, Stenotrophomonas, Stomatobaculum, Streptococcus, Syntrophomonadaceae, Tannerella, Treponema, Variovorax, Veillonella, Veillonellaceae, Weeksellaceae, and Yersinia.

In certain embodiments, the oral bacteria may be from a genus selected from the group consisting of Abiotrophia, Absconditabacteria, Achromobacter, Acidipropionibacterium, Acidovorax, Aeriscardovia, Afipia, Aggregatibacter, Agrobacterium, Alkalihalobacillus, Alloiococcus, Alloprevotella, Alloscardovia, Anaerococcus, Anaeroglobus, Anaerolineae, Anoxybacillus, Aquamicrobium, Arachnia, Arcanobacterium, Arsenicicoccus, Arthrospira, Atopobium, Bacteroidales, Bdellovibrio, Bosea, Bradyrhizobium, Brevibacterium, Brevundimonas, Brochothrix, Bulleidia, Butyrivibrio, Cardiobacterium, Catonella, Caulobacter, Cellulosimicrobium, Centipeda, Chryseobacterium, Cloacibacterium, Clostridiales, Colibacter, Comamonas, Cronobacter, Cryptobacterium, Cupriavidus, Cutibacterium, Delftia, Dermabacter, Desulfobulbus, Desulfomicrobium, Desulfovibrio, Dialister, Dietzia, Dolosigranulum, Eggerthella, Eggerthia, Enhydrobacter, Erysipelotrichaceae, Erythrobacter, Fannyhessea, Fastidiosipila, Filifactor, Finegoldia, Flavitalea, Fretibacterium, Gracilibacteria, Granulicatella, Haematobacter, Janibacter, Jeotgalicoccus, Johnsonella, Jonquetella, Kluyvera, Kocuria, Kytococcus, Lachnoanaerobaculum, Lachnospiraceae, Lacticaseibacillus, Lactiplantibacillus, Lancefieldella, Lautropia, Lawsonella, Lentilactobacillus, Leptothrix, Leptotrichia, Levilactobacillus, Ligilactobacillus, Limosilactobacillus, Lysinibacillus, Megasphaera, Mesorhizobium, Micavibrio, Microbacterium, Mitsuokella, Mogibacterium, Mollicutes, Mycolicibacterium, Mycoplasmopsis, Novosphingobium, Odoribacter, Olegusella, Olsenella, Oribacterium, Oryzomicrobium, Ottowia, Paenibacillus, Paracoccus, Parascardovia, Parvimonas, Pedobacter, Peptidiphaga, Peptococcus, Peptoniphilaceae, Peptoniphilus, Phocaeicola, Propionibacteriaceae, Pseudoleptotrichia, Pseudoramibacter, Pyramidobacter, Ralstonia, Rhodobacter, Rhodopseudomonas, Riemerella, Roseomonas, Rothia, Saccharibacteria, Sanguibacter, Scardovia, Schaalia, Schlegelella, Segetibacter, Selenomonas, Shuttleworthia, Simonsiella, Slackia, Sneathia, Solobacterium, Sphingomonas, Stomatobaculum, Syntrophomonadaceae, Tannerella, Variovorax, Veillonellaceae, and Weeksellaceae.

In certain embodiments, the oral bacteria may be from a species selected from the group consisting of Porphyromonas gingivalis, Streptococcus mutans, Fusobacterium nucleatum, Aggregatibacter actinomycetemcomitans, Escherichia coli, Klebsiella pneumoniae, Treponema denticola, Tannerella forsythia, Pseudomonas aeruginosa, Acinetobacter baumannii, Klebsiella oxytoca, Staphylococcus aureus, Enterococcus faecalis, Enterococcus faecium, Lactobacilli fermentum, Lactobacilli rhamnosus, Lactobacilli gasseri, Lactobacilli casei, Lactobacilli salivarius, Streptococcus gordonii, Streptococcus parasanguinis, Streptococcus pyogenes, Actinomyces naeslundii, Prevotella denticola, Campylobacter sputorum, Saccharibacteria HMT-356, Selenomonas sputigena, Corynebacterium durum, Corynebacterium matruchotii, Leptotrichia HMT-498, Leptotrichia HMT-221, Leptotrichia HMT-417, Leptotrichia wadei, Leptotrichia buccalis, Leptotrichia trevisanii, Veillonella atypica, Veillonella parvula, dispar, Haemophilius influenzae, Prevotella nigrescens, Solobacterium moorei, Atopobium parvulum, Eubacterium sulci, Filifactor alocis, Scardovia wiggsiae, and Helicobacter pylori.

In certain embodiments, the oral bacteria may be from a species selected from the group consisting of Bartonella schoenbuchensis, Caulobacter sp. HMT-002, Sphingomonas glacialis, Sphingomonas sp. HMT-004, Acinetobacter Iwoffii, Novosphingobium humi, Erythrobacter tepidarius, Mogibacterium vescum, Acinetobacter radioresistens, Eikenella sp. HMT-011, Kingella sp. HMT-012, Neisseria bacilliformis, Neisseria oralis, Anaerococcus octavius, Neisseria sp. HMT-018, Corynebacterium accolens, Neisseria sp. HMT-020, Streptococcus vestibularis, Lautropia mirabilis, Delftia acidovorans, Schlegelella thermodepolymerans, Leptothrix sp. HMT-025, Schlegelella aquatica, Cupriavidus gilardii, Oryzomicrobium terrae, Treponema vincentii, Corynebacterium afermentans, Corynebacterium amycolatum, Pseudomonas sp. HMT-032, Corynebacterium appendicis, Corynebacterium aurimucosum, Haemophilus paraphrohaemolyticus, Haemophilus sp. HMT-036, Stenotrophomonas nitritireducens, Olsenella uli, Bdellovibrio sp. HMT-039, Desulfovibrio sp. HMT-040, Desulfobulbus sp. HMT-041, Mogibacterium timidum, Campylobacter sp. HMT-044, Alkalihalobacillus clausih, Gemella morbillorum, Corynebacterium jeikeium, Paenibacillus typhae, Corynebacterium kroppenstedtii, Corynebacterium macginleyi, Limosilactobacillus vaginalis, Limosilactobacillus sp. HMT-052, Corynebacterium minutissimum, Corynebacterium pilbarense, Streptococcus sp. HMT-056, Streptococcus sp. HMT-057, Streptococcus oralis, Corynebacterium propinquum, Corynebacterium pseudodiphtheriticum, Streptococcus sp. HMT-061, Corynebacterium simulans, Corynebacterium singulare, Streptococcus sp. HMT-064, Streptococcus sp. HMT-066, Corynebacterium striatum, Streptococcus australis, Streptococcus sp. HMT-074, Ruminococcaceae bacterium HMT-075, Staphylococcus warneri, Corynebacterium tuberculostearicum, Oribacterium sp. HMT-078, Butyrivibrio sp. HMT-080, Peptostreptococcaceae bacterium HMT-081, Lachnoanaerobaculum orale, Lachnoanaerobaculum sp. HMT-083, Kocuria palustris, Ruminococcaceae bacterium HMT-085, Lachnospiraceae bacterium HMT-086, Micrococcus luteus, Lachnospiraceae bacterium HMT-088, Lachnoanaerobaculum sp. HMT-089, Butyrivibrio sp. HMT-090, Peptostreptococcaceae bacterium HMT-091, Neisseria weaveri, Clostridiales bacterium HMT-093, Lachnospiraceae bacterium HMT-094, Shuttleworthia satelles, Lachnospiraceae bacterium HMT-096, Stomatobaculum sp. HMT-097, Moraxella nonliquefaciens, Neisseria macacae, Lachnospiraceae bacterium HMT-100, Neisseria perflava, Oribacterium sp. HMT-102, Peptostreptococcaceae bacterium HMT-103, Paracoccus yeei, Peptostreptococcaceae [Eubacterium] infirmum, Peptostreptococcaceae [Eubacterium] yurii, Lachnoanaerobaculum umeaense, Oribacterium asaccharolyticum, Peptoniphilus harei, Parvimonas sp. HMT-110, Parvimonas micra, Peptostreptococcus stomatis, Peptoniphilaceae bacterium HMT-113, Cutibacterium granulosum, Serratia marcescens, Staphylococcus capitis, Staphylococcus cohnii, Dialister invisus, Dialister sp. HMT-119, Staphylococcus haemolyticus, Anaeroglobus geminatus, Megasphaera micronuciformis, Colibacter massiliensis, Selenomonas artemidis, Selenomonas flueggei, Selenomonas sp. HMT-126, Staphylococcus hominis, Staphylococcus lugdunensis, Veillonellaceae bacterium HMT-129, Selenomonas noxia, Mitsuokella sp. HMT-131, Veillonellaceae bacterium HMT-132, Selenomonas sp. HMT-133, Selenomonas sp. HMT-134, Veillonellaceae bacterium HMT-135, Selenomonas sp. HMT-136, Selenomonas sp. HMT-137, Selenomonas sp. HMT-138, Selenomonas dianae, Staphylococcus pasteuri, Staphylococcus pettenkoferi, Veillonellaceae bacterium HMT-145, Selenomonas sp. HMT-146, Veillonellaceae bacterium HMT-148, Selenomonas sp. HMT-149, Veillonellaceae bacterium HMT-150, Selenomonas sputigena, Streptococcus thermophilus, Klebsiella aerogenes, Moraxella lincolnii, Veillonellaceae bacterium HMT-155, Veillonella rogosae, Veillonella dispar, Veillonella parvula, Lachnospiraceae bacterium HMT-163, Catonella sp. HMT-164, Catonella morbi, Johnsonella sp. HMT-166, Peptococcus sp. HMT-167, Peptococcus sp. HMT-168, Actinomyces sp. HMT-169, Actinomyces sp. HMT-170, Actinomyces sp. HMT-171, Schaalia sp. HMT-172, Lawsonella clevelandensis, Neisseriaceae bacterium HMT-174, Actinomyces sp. HMT-175, Actinomyces naeslundii, Schaalia sp. HMT-178, Actinomyces timonensis, Schaalia sp. HMT-180, Schaalia lingnae, Anoxybacillus flavithermus, Peptidiphaga sp. HMT-183, Corynebacterium tuscaniense, Microbacterium ginsengisoli, Microbacterium flavescens, Peptoniphilus sp. HMT-187, Rothia aeria, Kocuria atrinae, Arsenicicoccus bolidensis, Acidipropionibacterium acidifaciens, Propionibacteriaceae bacterium HMT-192, Cutibacterium sp. HMT-193, Arachnia rubra, Scardovia wiggsiae, Enhydrobacter aerosaccus, Kocuria rhizophila, Alloscardovia omnicolens, Lancefieldella sp. HMT-199, Fusobacterium nucleatum, Fusobacterium periodonticum, Fusobacterium sp. HMT-203, Fusobacterium sp. HMT-204, Fusobacterium sp. HMT-205, Cloacibacterium sp. HMT-206, Corynebacterium gottingense, Corynebacterium bovis, Acidovorax ebreus, Acidovorax caeni, Leptotrichia sp. HMT-212, Leptotrichia hongkongensis, Leptotrichia shahii, Leptotrichia sp. HMT-215, Acidovorax temperans, Leptotrichia sp. HMT-217, Leptotrichia sp. HMT-218, Pseudoleptotrichia sp. HMT-219, Pseudoleptotrichia sp. HMT-221, Leptotrichia wadei, Leptotrichia sp. HMT-223, Leptotrichia hofstadii, Leptotrichia sp. HMT-225, Treponema sp. HMT-226, Treponema sp. HMT-227, Treponema sp. HMT-228, Arthrospira platensis, Treponema sp. HMT-230, Treponema sp. HMT-231, Treponema sp. HMT-232, Staphylococcus schleiferi, Treponema sp. HMT-234, Treponema sp. HMT-235, Treponema sp. HMT-236, Treponema sp. HMT-237, Treponema sp. HMT-238, Treponema sp. HMT-239, Brochothrix thermosphacta, Treponema sp. HMT-242, Jeotgalicoccus huakuii, Roseomonas gilardii, Roseomonas mucosa, Treponema sp. HMT-246, Treponema sp. HMT-247, Fusobacterium sp. HMT-248, Treponema sp. HMT-249, Treponema sp. HMT-250, Treponema sp. HMT-251, Treponema sp. HMT-252, Treponema sp. HMT-253, Treponema sp. HMT-254, Treponema sp. HMT-256, Treponema sp. HMT-257, Treponema sp. HMT-258, Haemophilus sp. HMT-259, Treponema sp. HMT-260, Segetibacter aerophilus, Treponema sp. HMT-262, Treponema sp. HMT-263, Treponema sp. HMT-264, Treponema sp. HMT-265, Leptothrix sp. HMT-266, Treponema sp. HMT-268, Treponema sp. HMT-269, Treponema sp. HMT-270, Treponema sp. HMT-271, Phocaeicola abscessus, Porphyromonas endodontalis, Bacteroidales bacterium HMT-274, Porphyromonas sp. HMT-275, Moraxella sp. HMT-276, Porphyromonas sp. HMT-277, Porphyromonas sp. HMT-278, Porphyromonas pasteri, Bacteroidetes bacterium HMT-280, Bacteroidetes bacterium HMT-281, Acinetobacter junii, Porphyromonas catoniae, Porphyromonas sp. HMT-284, Porphyromonas sp. HMT-285, Tannerella sp. HMT-286, Prevotella oulorum, Prevotella maculosa, Anaerococcus sp. HMT-290, Prevotella denticola, Anaerococcus sp. HMT-294, Anaerococcus sp. HMT-295, Acinetobacter johnsonii, Prevotella histicola, Prevotella nanceiensis, Prevotella sp. HMT-300, Prevotella sp. HMT-301, Alloprevotella rava, Prevotella pleuritidis, Prevotella sp. HMT-304, Prevotella sp. HMT-305, Prevotella sp. HMT-306, Prevotella salivae, Alloprevotella sp. HMT-308, Prevotella sp. HMT-309, Prevotella oris, Aerococcus viridans, Prevotella jejuni, Prevotella sp. HMT-314, Prevotella sp. HMT-315, Haematobacter missouriensis, Prevotella sp. HMT-317, Pedobacter sp. HMT-318, Chryseobacterium sp. HMT-319, Flavitalea sp. HMT-320, Pedobacter sp. HMT-321, Riemerella sp. HMT-322, Capnocytophaga sp. HMT-323, Capnocytophaga sp. HMT-324, Capnocytophaga granulosa, Capnocytophaga endodontalis, Neisseriaceae bacterium HMT-327, Corynebacterium mastitidis, Capnocytophaga leadbetteri, Micavibrio aeruginosavorus, Staphylococcus auricularis, Capnocytophaga sp. HMT-332, Corynebacterium massiliense, Capnocytophaga sp. HMT-334, Capnocytophaga sp. HMT-335, Capnocytophaga sp. HMT-336, Capnocytophaga gingivalis, Capnocytophaga sp. HMT-338, Janibacterindicus, Brevibacterium paucivorans, Corynebacterium coyleae, Novosphingobium panipatense, Achromobacter xylosoxidans, Pseudomonas luteola, Absconditabacteria bacterium HMT-345, Saccharibacteria bacterium HMT-346, Saccharibacteria bacterium HMT-347, Saccharibacteria bacterium HMT-348, Saccharibacteria bacterium HMT-349, Saccharibacteria bacterium HMT-350, Saccharibacteria bacterium HMT-351, Saccharibacteria bacterium HMT-352, Saccharibacteria bacterium HMT-353, Dermabacter hominis, Saccharibacteria bacterium HMT-355, Saccharibacteria bacterium HMT-356, Pyramidobacter piscolens, Fretibacterium sp. HMT-358, Fretibacterium sp. HMT-359, Fretibacterium sp. HMT-360, Fretibacterium sp. HMT-361, Fretibacterium sp. HMT-362, Fretibacterium fastidiosum, Saccharibacteria bacterium HMT-364, Bacteroidetes bacterium HMT-365, Ruminococcaceae bacterium HMT-366, Saccharibacteria bacterium HMT-367, Dietzia cinnamea, Peptostreptococcaceae bacterium HMT-369, Fusobacterium sp. HMT-370, Saccharibacteria bacterium HMT-371, Stomatobaculum sp. HMT-373, Cellulosimicrobium cellulans, Peptoniphilus sp. HMT-375, Prevotella sp. HMT-376, Prevotella micans, Capnocytophaga sp. HMT-380, Ruminococcaceae bacterium HMT-381, Peptostreptococcaceae bacterium HMT-382, Peptostreptococcaceae bacterium HMT-383, Peptoniphilus sp. HMT-386, Selenomonas sp. HMT-388, Abiotrophia defectiva, Leptotrichia sp. HMT-392, Parvimonas sp. HMT-393, Prevotella sp. HMT-396, Clostridiales bacterium HMT-402, Ralstonia sp. HMT-406, Aeriscardovia bacterium HMT-407, Acinetobacter sp. HMT-408, Streptococcus parasanguinis, Capnocytophaga sp. HMT-412, Actinomyces sp. HMT-414, Fannyhessea sp. HMT-416, Leptotrichia sp. HMT-417, Lentilactobacillus parafarraginis, Stomatobaculum longum, Cloacibacterium normanense, Streptococcus sp. HMT-423, Lentilactobacillus kisonensis, Streptococcus infantis, Syntrophomonadaceae bacterium HMT-435, Bacteroidetes bacterium HMT-436, Anaerolineae bacterium HMT-439, Selenomonas sp. HMT-442, Prevotella sp. HMT-443, Actinomyces sp. HMT-448, Catonella sp. HMT-451, Butyrivibrio sp. HMT-455, Oribacterium sinus, Aggregatibacter sp. HMT-458, Kingella sp. HMT-459, Lactobacillus ultunensis, Leptotrichia sp. HMT-463, Bacteroides zoogleoformans, Alloprevotella tannerae, Peptostreptococcaceae [Eubacterium] sulci, Bacillus subtilis, Prevotella melaninogenica, Prevotella sp. HMT-472, Alloprevotella sp. HMT-473, Prevotella sp. HMT-475, Neisseria subflava, Pseudomonas stutzeri, Selenomonas sp. HMT-478, Selenomonas sp. HMT-479, Selenomonas sp. HMT-481, Veillonellaceae bacterium HMT-483, Erysipelothrix tonsillarum, Agrobacterium tumefaciens, Saccharibacteria bacterium HMT-488, Treponema sp. HMT-490, Peptostreptococcaceae bacterium HMT-493, Lachnoanaerobaculum saburreum, Peptostreptococcaceae bacterium HMT-495, Lachnoanaerobaculum sp. HMT-496, Leptotrichia sp. HMT-498, Neisseria sp. HMT-499, Lachnospiraceae bacterium HMT-500, Selenomonas sp. HMT-501, Bacteroidetes bacterium HMT-503, Mollicutes bacterium HMT-504, Bacteroidetes bacterium HMT-505, Bacteroidetes bacterium HMT-507, Treponema sp. HMT-508, Bacteroidetes bacterium HMT-509, Bacteroidetes bacterium HMT-511, Aggregatibacter sp. HMT-512, Aggregatibacter sp. HMT-513, Prevotella sp. HMT-515, Odoribacter bacterium HMT-516, Treponema sp. HMT-517, Treponema sp. HMT-518, Mitsuokella sp. HMT-521, Neisseria sp. HMT-523, Veillonella atypica, Actinomyces sp. HMT-525, Prevotella koreensis, Lactobacillus acidophilus, Cutibacterium acnes, Aggregatibacter actinomycetemcomitans, Granulicatella adiacens, Haemophilus aegyptius, Pseudomonas aeruginosa, Streptococcus agalactiae, Pseudoramibacter alactolyticus, Filifactor alocis, Cardiobacterium valvarum, Treponema amylovorum, Peptostreptococcus anaerobius, Streptococcus anginosus, Brucella anthropi, Aggregatibacter aphrophilus, Porphyromonas asaccharolytica, Peptoniphilus asaccharolyticus, Staphylococcus aureus, Cutibacterium avidum, Prevotella baroniae, Acinetobacter baumannii, Gemella bergeri, Prevotella bivia, Peptostreptococcaceae [Eubacterium] brachy, Levilactobacillus brevis, Afipia broomeae, Prevotella buccae, Mycoplasma buccale, Prevotella buccalis, Leptotrichia buccalis, Enterobacter cancerogenus, Staphylococcus caprae, Lacticaseibacillus casei, Eggerthia catenaformis, Burkholderia cepacia, Prevotella veroralis, Escherichia coli, Campylobacter concisus, Streptococcus constellatus, Eikenella corrodens, Streptococcus cristatus, Cryptobacterium curtum, Campylobacter curvus, Kingella denitrificans, Prevotella dentalis, Treponema denticola, Parascardovia denticolens, Rothia dentocariosa, Bifidobacterium dentium, Brevundimonas diminuta, Corynebacterium diphtheriae, Mogibacterium diversum, Streptococcus downei, Corynebacterium durum, Granulicatella elegans, Bradyrhizobium elkanii, Neisseria elongata, Prevotella enoeca, Staphylococcus epidermidis, Slackia exigua, Bulleidia extructa, Enterococcus faecalis, Desulfovibrio fairfieldensis, Mycoplasma faucium, Mycoplasmopsis fermentans, Limosilactobacillus fermentum, Neisseria flava, Neisseria flavescens, Pseudomonas fluorescens, Tannerella forsythia, Lysinibacillus fusiformis, Lactobacillus gasseri, Mycoplasma genitalium, Schaalia georgiae, Actinomyces gerencseriae, Porphyromonas gingivalis, Neisseria gonorrhoeae, Streptococcus gordonii, Campylobacter gracilis, Gemella haemolysans, Capnocytophaga haemolytica, Bacteroides heparinolyticus, Mycoplasma hominis, Cardiobacterium hominis, Enterobacter hormaechei, Johnsonella ignava, Bosea vestrisii, Selenomonas infelix, Haemophilus influenzae, Scardovia inopinata, Prevotella intermedia, Streptococcus intermedius, Actinomyces israelii, Kingella kingae, Neisseria lactamica, Rhodopseudomonas telluris, Treponema lecithinolyticum, Eggerthella lenta, Eubacterium limosum, Mycoplasmopsis lipophila, Prevotella loescheii, Mesorhizobium japonicum, Aquamicrobium lusatiense, Finegoldia magna, Stenotrophomonas maltophilia, Treponema maltophilum, Prevotella marshii, Corynebacterium matruchotii, Treponema medium, Neisseria meningitidis, Schaalia meyeri, Peptostreptococcaceae [Eubacterium] minutum, Atopobium minutum, Proteus mirabilis, Streptococcus mitis, Solobacterium moorei, Rothia mucilaginosa, Neisseria mucosa, Simonsiella muelleri, Mitsuokella multacida, Prevotella multiformis, Streptococcus mutans, Actinomyces viscosus, Fusobacterium naviforme, Fusobacterium necrophorum, Mogibacterium neglectum, Mycolicibacterium neoaurum, Prevotella nigrescens, Peptostreptococcaceae [Eubacterium] nodatum, Capnocytophaga ochracea, Schaalia odontolytica, Desulfomicrobium orale, Mycoplasma orale, Prevotella oralis, Kingella oralis, Actinomyces oricola, Limosilactobacillus oris, Moraxella osloensis, Prevotella pallens, Lacticaseibacillus paracasei, Variovorax paradoxus, Haemophilus parainfluenzae, Aggregatibacter paraphrophilus, Lancefieldella parvula, Treponema parvum, Treponema pectinovorum, Centipeda periodontii, Streptococcus peroris, Klebsiella pneumoniae, Mycoplasma pneumoniae, Chlamydia pneumoniae, Streptococcus pneumoniae, Dialister pneumosintes, Neisseria polysaccharea, Anaerococcus prevotii, Arachnia propionica, Pseudomonas oleovorans, Mogibacterium pumilum, Treponema putidum, Streptococcus pyogenes, Actinomyces radicidentis, Erythrobacter ramosus, Campylobacter rectus, Lacticaseibacillus rhamnosus, Lancefieldella rimae, Cronobacter sakazakii, Mycoplasma salivarium, Streptococcus salivarius, Ligilactobacillus salivarius, Gemella sanguinis, Streptococcus sanguinis, Peptostreptococcaceae [Eubacterium] saphenum, Aggregatibacter segnis, Campylobacter showae, Neisseria sicca, Streptococcus sinensis, Streptococcus sobrinus, Treponema socranskii, Capnocytophaga sputigena, Campylobacter sputorum, Jonquetella anthropi, Veillonella sp. HMT-780, Prevotella saccharolytica, Prevotella fusca, Porphyromonas uenonis, Paenibacillus phoenicis, Bacteroides pyogenes, Anaerococcus tetradius, Peptoniphilaceae bacterium HMT-790, Prevotella multisaccharivorax, Prevotella shahii, Enterococcus casseliflavus, Enterococcus saccharolyticus, Enterococcus italicus, Lactococcus lactis, Treponema pallidum, Olsenella profusa, Olsenella sp. HMT-807, Tannerella sp. HMT-808, Olsenella sp. HMT-809, Olegusella massiliensis, Arcanobacterium haemolyticum, Helicobacter pylori, Dolosigranulum pigrum, Fannyhessea vaginae, Limosilactobacillus coleohominis, Lactobacillus crispatus, Limosilactobacillus reuteri, Lactobacillus johnsonii, Prevotella sp. HMT-820, Haemophilus ducreyi, Mycobacterium tuberculosis, Mycobacterium leprae, Bacillus anthracis, Listeria monocytogenes, Yersinia pestis, Bordetella pertussis, Gardnerella vaginalis, Mobiluncus mulieris, Alloiococcus otitis, Corynebacterium otitidis, Moraxella catarrhalis, Pseudomonas otitidis, Corynebacterium mucifaciens, Peptoniphilus lacrimalis, Sneathia sanguinegens, Lactobacillus iners, Lactobacillus jensenii, Peptoniphilus indolicus, Megasphaera sp. HMT-841, Campylobacterureolyticus, Dialister micraerophilus, Sneathia vaginalis, Pseudoleptotrichia goodfellowii, Leptotrichia sp. HMT-847, Peptidiphaga gingivicola, Actinomyces johnsonii, Schaalia cardiffensis, Haemophilus haemolyticus, Actinomyces massiliensis, Corynebacterium urealyticum, Ralstonia pickettii, Kytococcus sedentarius, Sanguibacter keddieii, Rhodobacter capsulatus, Comamonas testosteroni, Anaerococcus lactolyticus, Fusobacterium gonidiaformans, Lactiplantibacillus plantarum, Bifidobacterium longum, Capnocytophaga sp. HMT-863, Capnocytophaga sp. HMT-864, Kluyvera ascorbata, Actinomyces graevenitzii, Saccharibacteria bacterium HMT-869, Saccharibacteria bacterium HMT-870, Gracilibacteria bacterium HMT-871, Gracilibacteria bacterium HMT-872, Gracilibacteria bacterium HMT-873, Absconditabacteria bacterium HMT-874, Absconditabacteria bacterium HMT-875, Clostridiales bacterium HMT-876, Schaalia sp. HMT-877, Capnocytophaga sp. HMT-878, Leptotrichia sp. HMT-879, Enterococcus durans, Lentilactobacillus buchneri, Limosilactobacillus panis, Lactiplantibacillus pentosus, Lentilactobacillus rapi, Prevotella scopos, Veillonella denticariosi, Actinomyces dentalis, Bifidobacterium breve, Bifidobacterium subtile, Bifidobacterium scardovii, Selenomonas sp. HMT-892, Actinomyces oris, Ottowia sp. HMT-894, Bifidobacterium animalis, Actinomyces sp. HMT-896, Actinomyces sp. HMT-897, Aggregatibacter sp. HMT-898, Bacteroidetes bacterium HMT-899, Weeksellaceae sp. HMT-900, Capnocytophaga sp. HMT-901, Capnocytophaga sp. HMT-902, Capnocytophaga sp. HMT-903, Erysipelotrichaceae bacterium HMT-904, Erysipelotrichaceae bacterium HMT-905, Mollicutes bacterium HMT-906, Weeksellaceae sp. HMT-907, Haemophilus sp. HMT-908, Leptotrichia sp. HMT-909, Stomatobaculum sp. HMT-910, Bacteroidetes bacterium HMT-911, Alloprevotella sp. HMT-912, Alloprevotella sp. HMT-913, Alloprevotella sp. HMT-914, Propionibacteriaceae bacterium HMT-915, Tannerella sp. HMT-916, Veillonella sp. HMT-917, Veillonellaceae bacterium HMT-918, Selenomonas sp. HMT-919, Selenomonas sp. HMT-920, Paenibacillus glucanolyticus, Peptostreptococcaceae bacterium HMT-922, Lachnospiraceae bacterium HMT-924, Treponema sp. HMT-927, Gemella sp. HMT-928, Peptoniphilaceae bacterium HMT-929, Porphyromonas sp. HMT-930, Weeksellaceae sp. HMT-931, Kingella sp. HMT-932, Pedobacter sp. HMT-933, Oribacterium parvum, Fastidiosipila sanguinis, Selenomonas sp. HMT-936, Selenomonas sp. HMT-937, Olsenella sp. HMT-939, Prevotella sp. HMT-942, Prevotella aurantiaca, Haemophilus sputorum, Haemophilus parahaemolyticus, Haemophilus pittmaniae, Streptococcus lactarius, Aggregatibacter sp. HMT-949, Peptostreptococcaceae bacterium HMT-950, Treponema sp. HMT-951, Saccharibacteria bacterium HMT-952, Fusobacterium hwasookii, Saccharibacteria bacterium HMT-954, Saccharibacteria bacterium HMT-955, Neisseria cinerea, and Saccharibacteria bacterium HMT-957.

In certain embodiments, the oral bacteria may be from a species selected from the group consisting of Abiotrophia defectiva, Absconditabacteria bacterium HMT-345, Absconditabacteria bacterium HMT-874, Absconditabacteria bacterium HMT-875, Achromobacter xylosoxidans, Acidipropionibacterium acidifaciens, Acidovorax caeni, Acidovorax ebreus, Acidovorax temperans, Acinetobacter johnsonii, Acinetobacter junii, Acinetobacter Iwoffii, Acinetobacter radioresistens, Acinetobacter sp. HMT-408, Actinomyces dentalis, Actinomyces gerencseriae, Actinomyces graevenitzii, Actinomyces johnsonii, Actinomyces massiliensis, Actinomyces oricola, Actinomyces oris, Actinomyces radicidentis, Actinomyces sp. HMT-169, Actinomyces sp. HMT-170, Actinomyces sp. HMT-171, Actinomyces sp. HMT-175, Actinomyces sp. HMT-414, Actinomyces sp. HMT-448, Actinomyces sp. HMT-525, Actinomyces sp. HMT-896, Actinomyces sp. HMT-897, Actinomyces timonensis, Actinomyces viscosus, Aeriscardovia bacterium HMT-407, Aerococcus viridans, Afipia broomeae, Aggregatibacter aphrophilus, Aggregatibacter paraphrophilus, Aggregatibacter segnis, Aggregatibacter sp. HMT-458, Aggregatibacter sp. HMT-512, Aggregatibacter sp. HMT-513, Aggregatibacter sp. HMT-898, Aggregatibacter sp. HMT-949, Agrobacterium tumefaciens, Alkalihalobacillus clausii, Alloiococcus otitis, Alloprevotella rava, Alloprevotella sp. HMT-308, Alloprevotella sp. HMT-473, Alloprevotella sp. HMT-912, Alloprevotella sp. HMT-913, Alloprevotella sp. HMT-914, Alloprevotella tannerae, Alloscardovia omnicolens, Anaerococcus lactolyticus, Anaerococcus octavius, Anaerococcus prevotii, Anaerococcus sp. HMT-290, Anaerococcus sp. HMT-294, Anaerococcus sp. HMT-295, Anaerococcus tetradius, Anaeroglobus geminatus, Anaerolineae bacterium HMT-439, Anoxybacillus flavithermus, Aquamicrobium lusatiense, Arachnia propionica, Arachnia rubra, Arcanobacterium haemolyticum, Arsenicicoccus bolidensis, Arthrospira platensis, Atopobium minutum, Bacteroidales bacterium HMT-274, Bacteroides heparinolyticus, Bacteroides pyogenes, Bacteroides zoogleoformans, Bacteroidetes bacterium HMT-280, Bacteroidetes bacterium HMT-281, Bacteroidetes bacterium HMT-365, Bacteroidetes bacterium HMT-436, Bacteroidetes bacterium HMT-503, Bacteroidetes bacterium HMT-505, Bacteroidetes bacterium HMT-507, Bacteroidetes bacterium HMT-509, Bacteroidetes bacterium HMT-511, Bacteroidetes bacterium HMT-899, Bacteroidetes bacterium HMT-911, Bartonella schoenbuchensis, Bdellovibrio sp. HMT-039, Bifidobacterium animalis, Bifidobacterium breve, Bifidobacterium dentium, Bifidobacterium longum, Bifidobacterium scardovii, Bifidobacterium subtile, Bosea vestrisii, Bradyrhizobium elkanii, Brevibacterium paucivorans, Brevundimonas diminuta, Brochothrix thermosphacta, Brucella anthropi, Bulleidia extructa, Butyrivibrio sp. HMT-080, Butyrivibrio sp. HMT-090, Butyrivibrio sp. HMT-455, Campylobacter concisus, Campylobacter curvus, Campylobacter gracilis, Campylobacter rectus, Campylobacter showae, Campylobacter sp. HMT-044, Campylobacter sputorum, Campylobacter ureolyticus, Capnocytophaga endodontalis, Capnocytophaga gingivalis, Capnocytophaga granulosa, Capnocytophaga haemolytica, Capnocytophaga leadbetteri, Capnocytophaga ochracea, Capnocytophaga sp. HMT-323, Capnocytophaga sp. HMT-324, Capnocytophaga sp. HMT-332, Capnocytophaga sp. HMT-334, Capnocytophaga sp. HMT-335, Capnocytophaga sp. HMT-336, Capnocytophaga sp. HMT-338, Capnocytophaga sp. HMT-380, Capnocytophaga sp. HMT-412, Capnocytophaga sp. HMT-863, Capnocytophaga sp. HMT-864, Capnocytophaga sp. HMT-878, Capnocytophaga sp. HMT-901, Capnocytophaga sp. HMT-902, Capnocytophaga sp. HMT-903, Capnocytophaga sputigena, Cardiobacterium valvarum, Catonella morbi, Catonella sp. HMT-164, Catonella sp. HMT-451, Caulobacter sp. HMT-002, Cellulosimicrobium cellulans, Centipeda periodontii, Chlamydia pneumoniae, Chryseobacterium sp. HMT-319, Cloacibacterium normanense, Cloacibacterium sp. HMT-206, Clostridiales bacterium HMT-093, Clostridiales bacterium HMT-402, Clostridiales bacterium HMT-876, Colibacter massiliensis, Comamonas testosteroni, Corynebacterium accolens, Corynebacterium afermentans, Corynebacterium amycolatum, Corynebacterium appendicis, Corynebacterium aurimucosum, Corynebacterium bovis, Corynebacterium coyleae, Corynebacterium durum, Corynebacterium gottingense, Corynebacterium jeikeium, Corynebacterium kroppenstedtii, Corynebacterium macginleyi, Corynebacterium massiliense, Corynebacterium mastitidis, Corynebacterium matruchotii, Corynebacterium minutissimum, Corynebacterium mucifaciens, Corynebacterium otitidis, Corynebacterium pilbarense, Corynebacterium propinquum, Corynebacterium pseudodiphtheriticum, Corynebacterium simulans, Corynebacterium singulare, Corynebacterium striatum, Corynebacterium tuberculostearicum, Corynebacterium tuscaniense, Corynebacterium urealyticum, Cronobacter sakazakii, Cryptobacterium curtum, Cupriavidus gilardii, Cutibacterium acnes, Cutibacterium avidum, Cutibacterium granulosum, Cutibacterium sp. HMT-193, Delftia acidovorans, Dermabacter hominis, Desulfobulbus sp. HMT-041, Desulfomicrobium orale, Desulfovibrio fairfieldensis, Desulfovibrio sp. HMT-040, Dialister invisus, Dialister micraerophilus, Dialister pneumosintes, Dialister sp. HMT-119, Dietzia cinnamea, Dolosigranulum pigrum, Eggerthella lenta, Eggerthia catenaformis, Eikenella sp. HMT-011, Enhydrobacter aerosaccus, Enterobacter cancerogenus, Enterobacter hormaechei, Enterococcus casseliflavus, Enterococcus durans, Enterococcus italicus, Enterococcus saccharolyticus, Erysipelothrix tonsillarum, Erysipelotrichaceae bacterium HMT-904, Erysipelotrichaceae bacterium HMT-905, Erythrobacter ramosus, Erythrobacter tepidarius, Eubacterium limosum, Fannyhessea sp. HMT-416, Fannyhessea vaginae, Fastidiosipila sanguinis, Filifactor alocis, Finegoldia magna, Flavitalea sp. HMT-320, Fretibacterium fastidiosum, Fretibacterium sp. HMT-358, Fretibacterium sp. HMT-359, Fretibacterium sp. HMT-360, Fretibacterium sp. HMT-361, Fretibacterium sp. HMT-362, Fusobacterium gonidiaformans, Fusobacterium hwasookii, Fusobacterium naviforme, Fusobacterium necrophorum, Fusobacterium periodonticum, Fusobacterium sp. HMT-203, Fusobacterium sp. HMT-204, Fusobacterium sp. HMT-205, Fusobacterium sp. HMT-248, Fusobacterium sp. HMT-370, Gemella bergeri, Gemella haemolysans, Gemella sanguinis, Gemella sp. HMT-928, Gracilibacteria bacterium HMT-871, Gracilibacteria bacterium HMT-872, Gracilibacteria bacterium HMT-873, Granulicatella adiacens, Granulicatella elegans, Haematobacter missouriensis, Haemophilus paraphrohaemolyticus, Haemophilus pittmaniae, Haemophilus sp. HMT-036, Haemophilus sp. HMT-259, Haemophilus sp. HMT-908, Haemophilus sputorum, Janibacter indicus, Jeotgalicoccus huakuii, Johnsonella ignava, Johnsonella sp. HMT-166, Jonquetella anthropi, Kingella denitrificans, Kingella kingae, Kingella oralis, Kingella sp. HMT-012, Kingella sp. HMT-459, Kingella sp. HMT-932, Klebsiella aerogenes, Kluyvera ascorbata, Kocuria atrinae, Kocuria palustris, Kocuria rhizophila, Kytococcus sedentarius, Lachnoanaerobaculum orale, Lachnoanaerobaculum saburreum, Lachnoanaerobaculum sp. HMT-083, Lachnoanaerobaculum sp. HMT-089, Lachnoanaerobaculum sp. HMT-496, Lachnoanaerobaculum umeaense, Lachnospiraceae bacterium HMT-086, Lachnospiraceae bacterium HMT-088, Lachnospiraceae bacterium HMT-094, Lachnospiraceae bacterium HMT-096, Lachnospiraceae bacterium HMT-100, Lachnospiraceae bacterium HMT-163, Lachnospiraceae bacterium HMT-500, Lachnospiraceae bacterium HMT-924, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus crispatus, Lactobacillus gasseri, Lactobacillus iners, Lactobacillus jensenii, Lactobacillus johnsonii, Lactobacillus ultunensis, Lactococcus lactis, Lancefieldella parvula, Lancefieldella rimae, Lancefieldella sp. HMT-199, Lautropia mirabilis, Lawsonella clevelandensis, Lentilactobacillus buchneri, Lentilactobacillus kisonensis, Lentilactobacillus parafarraginis, Lentilactobacillus rapi, Leptothrix sp. HMT-025, Leptothrix sp. HMT-266, Leptotrichia buccalis, Leptotrichia hofstadii, Leptotrichia hongkongensis, Leptotrichia shahii, Leptotrichia sp. HMT-212, Leptotrichia sp. HMT-215, Leptotrichia sp. HMT-217, Leptotrichia sp. HMT-218, Leptotrichia sp. HMT-223, Leptotrichia sp. HMT-225, Leptotrichia sp. HMT-392, Leptotrichia sp. HMT-417, Leptotrichia sp. HMT-463, Leptotrichia sp. HMT-498, Leptotrichia sp. HMT-847, Leptotrichia sp. HMT-879, Leptotrichia sp. HMT-909, Leptotrichia wadei, Levilactobacillus brevis, Ligilactobacillus salivarius, Limosilactobacillus coleohominis, Limosilactobacillus fermentum, Limosilactobacillus oris, Limosilactobacillus panis, Limosilactobacillus reuteri, Limosilactobacillus sp. HMT-052, Limosilactobacillus vaginalis, Lysinibacillus fusiformis, Megasphaera micronuciformis, Megasphaera sp. HMT-841, Mesorhizobium japonicum, Micavibrio aeruginosavorus, Microbacterium flavescens, Microbacterium ginsengisoli, Micrococcus luteus, Mitsuokella multacida, Mitsuokella sp. HMT-131, Mitsuokella sp. HMT-521, Mobiluncus mulieris, Mogibacterium diversum, Mogibacterium neglectum, Mogibacterium pumilum, Mogibacterium timidum, Mogibacterium vescum, Mollicutes bacterium HMT-504, Mollicutes bacterium HMT-906, Moraxella lincolnii, Moraxella nonliquefaciens, Moraxella osloensis, Moraxella sp. HMT-276, Mycolicibacterium neoaurum, Mycoplasma buccale, Mycoplasma faucium, Mycoplasma orale, Mycoplasma salivarium, Mycoplasmopsis fermentans, Mycoplasmopsis lipophila, Neisseria bacilliformis, Neisseria cinerea, Neisseria elongata, Neisseria flava, Neisseria flavescens, Neisseria lactamica, Neisseria macacae, Neisseria mucosa, Neisseria oralis, Neisseria perflava, Neisseria polysaccharea, Neisseria sicca, Neisseria sp. HMT-018, Neisseria sp. HMT-020, Neisseria sp. HMT-499, Neisseria sp. HMT-523, Neisseria subflava, Neisseria weaveri, Neisseriaceae bacterium HMT-174, Neisseriaceae bacterium HMT-327, Novosphingobium humi, Novosphingobium panipatense, Odoribacter bacterium HMT-516, Olegusella massiliensis, Olsenella profusa, Olsenella sp. HMT-807, Olsenella sp. HMT-809, Olsenella sp. HMT-939, Olsenella uli, Oribacterium asaccharolyticum, Oribacterium parvum, Oribacterium sinus, Oribacterium sp. HMT-078, Oribacterium sp. HMT-102, Oryzomicrobium terrae, Ottowia sp. HMT-894, Paenibacillus glucanolyticus, Paenibacillus phoenicis, Paenibacillus typhae, Paracoccus yeei, Parascardovia denticolens, Parvimonas micra, Parvimonas sp. HMT-110, Parvimonas sp. HMT-393, Pedobacter sp. HMT-318, Pedobacter sp. HMT-321, Pedobacter sp. HMT-933, Peptidiphaga gingivicola, Peptidiphaga sp. HMT-183, Peptococcus sp. HMT-167, Peptococcus sp. HMT-168, Peptoniphilaceae bacterium HMT-113, Peptoniphilaceae bacterium HMT-790, Peptoniphilaceae bacterium HMT-929, Peptoniphilus asaccharolyticus, Peptoniphilus harei, Peptoniphilus indolicus, Peptoniphilus lacrimalis, Peptoniphilus sp. HMT-187, Peptoniphilus sp. HMT-375, Peptoniphilus sp. HMT-386, Peptostreptococcaceae [Eubacterium] brachy, Peptostreptococcaceae [Eubacterium] infirmum, Peptostreptococcaceae [Eubacterium] minutum, Peptostreptococcaceae [Eubacterium] nodatum, Peptostreptococcaceae [Eubacterium] saphenum, Peptostreptococcaceae [Eubacterium] sulci, Peptostreptococcaceae [Eubacterium] yurii, Peptostreptococcaceae bacterium HMT-081, Peptostreptococcaceae bacterium HMT-091, Peptostreptococcaceae bacterium HMT-103, Peptostreptococcaceae bacterium HMT-369, Peptostreptococcaceae bacterium HMT-382, Peptostreptococcaceae bacterium HMT-383, Peptostreptococcaceae bacterium HMT-493, Peptostreptococcaceae bacterium HMT-495, Peptostreptococcaceae bacterium HMT-922, Peptostreptococcaceae bacterium HMT-950, Peptostreptococcus anaerobius, Peptostreptococcus stomatis, Phocaeicola abscessus, Porphyromonas asaccharolytica, Porphyromonas catoniae, Porphyromonas endodontalis, Porphyromonas gingivalis, Porphyromonas pasteri, Porphyromonas sp. HMT-275, Porphyromonas sp. HMT-277, Porphyromonas sp. HMT-278, Porphyromonas sp. HMT-284, Porphyromonas sp. HMT-285, Porphyromonas sp. HMT-930, Porphyromonas uenonis, Prevotella aurantiaca, Prevotella baroniae, Prevotella bivia, Prevotella buccae, Prevotella buccalis, Prevotella dentalis, Prevotella denticola, Prevotella enoeca, Prevotella fusca, Prevotella histicola, Prevotella intermedia, Prevotella jejuni, Prevotella koreensis, Prevotella loescheii, Prevotella maculosa, Prevotella marshii, Prevotella micans, Prevotella multiformis, Prevotella multisaccharivorax, Prevotella nanceiensis, Prevotella nigrescens, Prevotella oralis, Prevotella oris, Prevotella oulorum, Prevotella pallens, Prevotella pleuritidis, Prevotella saccharolytica, Prevotella salivae, Prevotella scopos, Prevotella shahii, Prevotella sp. HMT-300, Prevotella sp. HMT-301, Prevotella sp. HMT-304, Prevotella sp. HMT-305, Prevotella sp. HMT-306, Prevotella sp. HMT-309, Prevotella sp. HMT-314, Prevotella sp. HMT-315, Prevotella sp. HMT-317, Prevotella sp. HMT-376, Prevotella sp. HMT-396, Prevotella sp. HMT-443, Prevotella sp. HMT-472, Prevotella sp. HMT-475, Prevotella sp. HMT-515, Prevotella sp. HMT-820, Prevotella sp. HMT-942, Prevotella veroralis, Propionibacteriaceae bacterium HMT-192, Propionibacteriaceae bacterium HMT-915, Pseudoleptotrichia goodfellowii, Pseudoleptotrichia sp. HMT-219, Pseudoleptotrichia sp. HMT-221, Pseudomonas fluorescens, Pseudomonas luteola, Pseudomonas oleovorans, Pseudomonas otitidis, Pseudomonas sp. HMT-032, Pseudomonas stutzeri, Pseudoramibacter alactolyticus, Pyramidobacter piscolens, Ralstonia pickettii, Ralstonia sp. HMT-406, Rhodobacter capsulatus, Rhodopseudomonas telluris, Riemerella sp. HMT-322, Roseomonas gilardii, Roseomonas mucosa, Rothia aeria, Rothia dentocariosa, Rothia mucilaginosa, Ruminococcaceae bacterium HMT-075, Ruminococcaceae bacterium HMT-085, Ruminococcaceae bacterium HMT-366, Ruminococcaceae bacterium HMT-381, Saccharibacteria bacterium HMT-346, Saccharibacteria bacterium HMT-347, Saccharibacteria bacterium HMT-348, Saccharibacteria bacterium HMT-349, Saccharibacteria bacterium HMT-350, Saccharibacteria bacterium HMT-351, Saccharibacteria bacterium HMT-352, Saccharibacteria bacterium HMT-353, Saccharibacteria bacterium HMT-355, Saccharibacteria bacterium HMT-356, Saccharibacteria bacterium HMT-364, Saccharibacteria bacterium HMT-367, Saccharibacteria bacterium HMT-371, Saccharibacteria bacterium HMT-488, Saccharibacteria bacterium HMT-869, Saccharibacteria bacterium HMT-870, Saccharibacteria bacterium HMT-952, Saccharibacteria bacterium HMT-954, Saccharibacteria bacterium HMT-955, Saccharibacteria bacterium HMT-957, Sanguibacter keddieii, Scardovia inopinata, Scardovia wiggsiae, Schaalia cardiffensis, Schaalia georgiae, Schaalia lingnae, Schaalia meyeri, Schaalia odontolytica, Schaalia sp. HMT-172, Schaalia sp. HMT-178, Schaalia sp. HMT-180, Schaalia sp. HMT-877, Schlegelella aquatica, Schlegelella thermodepolymerans, Segetibacter aerophilus, Selenomonas artemidis, Selenomonas dianae, Selenomonas flueggei, Selenomonas infelix, Selenomonas noxia, Selenomonas sp. HMT-126, Selenomonas sp. HMT-133, Selenomonas sp. HMT-134, Selenomonas sp. HMT-136, Selenomonas sp. HMT-137, Selenomonas sp. HMT-138, Selenomonas sp. HMT-146, Selenomonas sp. HMT-149, Selenomonas sp. HMT-388, Selenomonas sp. HMT-442, Selenomonas sp. HMT-478, Selenomonas sp. HMT-479, Selenomonas sp. HMT-481, Selenomonas sp. HMT-501, Selenomonas sp. HMT-892, Selenomonas sp. HMT-919, Selenomonas sp. HMT-920, Selenomonas sp. HMT-936, Selenomonas sp. HMT-937, Selenomonas sputigena, Shuttleworthia satelles, Simonsiella muelleri, Slackia exigua, Sneathia sanguinegens, Sneathia vaginalis, Solobacterium moorei, Sphingomonas glacialis, Sphingomonas sp. HMT-004, Staphylococcus auricularis, Staphylococcus capitis, Staphylococcus caprae, Staphylococcus cohnii, Staphylococcus hominis, Staphylococcus lugdunensis, Staphylococcus pasteuri, Staphylococcus pettenkoferi, Staphylococcus schleiferi, Staphylococcus warneri, Stenotrophomonas nitritireducens, Stomatobaculum longum, Stomatobaculum sp. HMT-097, Stomatobaculum sp. HMT-373, Stomatobaculum sp. HMT-910, Streptococcus australis, Streptococcus constellatus, Streptococcus cristatus, Streptococcus downei, Streptococcus gordonii, Streptococcus infantis, Streptococcus intermedius, Streptococcus lactarius, Streptococcus mitis, Streptococcus oralis, Streptococcus parasanguinis, Streptococcus peroris, Streptococcus salivarius, Streptococcus sanguinis, Streptococcus sinensis, Streptococcus sobrinus, Streptococcus sp. HMT-056, Streptococcus sp. HMT-057, Streptococcus sp. HMT-061, Streptococcus sp. HMT-064, Streptococcus sp. HMT-066, Streptococcus sp. HMT-074, Streptococcus sp. HMT-423, Streptococcus thermophilus, Streptococcus vestibularis, Syntrophomonadaceae bacterium HMT-435, Tannerella forsythia, Tannerella sp. HMT-286, Tannerella sp. HMT-808, Tannerella sp. HMT-916, Treponema amylovorum, Treponema denticola, Treponema lecithinolyticum, Treponema maltophilum, Treponema medium, Treponema parvum, Treponema pectinovorum, Treponema putidum, Treponema socranskii, Treponema sp. HMT-226, Treponema sp. HMT-227, Treponema sp. HMT-228, Treponema sp. HMT-230, Treponema sp. HMT-231, Treponema sp. HMT-232, Treponema sp. HMT-234, Treponema sp. HMT-235, Treponema sp. HMT-236, Treponema sp. HMT-237, Treponema sp. HMT-238, Treponema sp. HMT-239, Treponema sp. HMT-242, Treponema sp. HMT-246, Treponema sp. HMT-247, Treponema sp. HMT-249, Treponema sp. HMT-250, Treponema sp. HMT-251, Treponema sp. HMT-252, Treponema sp. HMT-253, Treponema sp. HMT-254, Treponema sp. HMT-256, Treponema sp. HMT-257, Treponema sp. HMT-258, Treponema sp. HMT-260, Treponema sp. HMT-262, Treponema sp. HMT-263, Treponema sp. HMT-264, Treponema sp. HMT-265, Treponema sp. HMT-268, Treponema sp. HMT-269, Treponema sp. HMT-270, Treponema sp. HMT-271, Treponema sp. HMT-490, Treponema sp. HMT-508, Treponema sp. HMT-517, Treponema sp. HMT-518, Treponema sp. HMT-927, Treponema sp. HMT-951, Treponema vincentii, Variovorax paradoxus, Veillonella atypica, Veillonella denticariosi, Veillonella dispar, Veillonella parvula, Veillonella rogosae, Veillonella sp. HMT-780, Veillonella sp. HMT-917, Veillonellaceae bacterium HMT-129, Veillonellaceae bacterium HMT-132, Veillonellaceae bacterium HMT-135, Veillonellaceae bacterium HMT-145, Veillonellaceae bacterium HMT-148, Veillonellaceae bacterium HMT-150, Veillonellaceae bacterium HMT-155, Veillonellaceae bacterium HMT-483, Veillonellaceae bacterium HMT-918, Weeksellaceae sp. HMT-900, Weeksellaceae sp. HMT-907, and Weeksellaceae sp. HMT-931.

In certain embodiments, the oral bacteria may be any of the oral bacteria set forth in Table 1.

TABLE 1
Oral bacteria

| Bacterial species | | |
|---|---|---|
| name | Brief description | Disease type |
| Porphyromonas | Periodontopathic pathogen that | Gum, |
| gingivalis | initiate and cause periodontitis. | systemic |
| Streptococcus | Acid producing biofilm bacteria | Cavities |
| mutans | that cause caries. | |
| Fusobacterium | Periodontal bridge organism | Gum, cancer, |
| nucleatum | that recruits early and late | systemic |
| | colonizers. | |
| Aggregatibacter | Periodontal pathogen that is | Gum |
| actinomycetemcomitans | associated with aggressive | |
| | periodontitis and has various | |
| | capability to invade the human | |
| | immune system. | |
| Escherichia coli | Low abundance oral bacteria | Gum, |
| | that has the potential to cause | systemic |
| | various inflammatory diseases. | |
| Klebsiella pneumoniae | Low abundance and low | Systemic |
| | prevalence bacteria that use oral | |
| | cavity and nostril as a resevoir | |
| | to cause pneumoniae, bacteremia | |
| | and nasal infection. One of the | |
| | ESKAPE pathogens that has | |
| | high rate of antibiotic resistance. | |
| Treponema denticola | Periodontal pathogen that is part | Gum |
| | of the red complex of bacterial | |
| | group that increase in abundance | |
| | and associated with the disease. | |
| Tannerellla forsythia | Part of the red complex of | Gum |
| | bacteria in periodontitis. Also | |
| | involved in peripheral infection | |
| | near periodontal lesions. | |
| Pseudomonas | Well known human pathogen | Gum, |
| aeruginosa | that is detected in oral cavity. | Systemic |
| | Involved in periodontal | |
| | diseases as well as pneumonia, | |
| | UTI and soft tissue infection | |
| Acinetobacter | Well known human pathogen | Gum, |
| baumannii | that is detected in oral cavity. | systemic |
| | One of the ESKAPE pathogens | |
| | that has high rate of antibiotic | |
| | resistance. Pneumonia, | |
| | bacteremia, UTI | |
| Klebsiella oxytoca | Closely related to K. pneumoniae | Gum, cancer |
| | One of the ESKAPE pathogens | |
| | that has high rate of antibiotic | |
| | resistance. Detected and isolated | |
| | from human oral cavity. Infection | |
| | can result in colitis and sepsis. | |
| Staphylococcus aureus | Commonly detected and isolated | Gum, |
| | from Human oral cavity. One of | systemic |
| | the ESKAPE pathogens that has | |
| | high rate of antibiotic resistance. | |
| | Skin and airway infections. | |
| Enterococcus faecalis | Colonize human Oral, GI and | Gum |
| | vaginal sites. Very resilient | |
| | bacteria can cause periodontitis, | |
| | bacteremia and UTI. | |
| Enterococcus faecium | Close relative to E. faecalis and | Gum |
| | cause similar diseases | |
| Lactobacilli fermentum, | Produce acid to break down | Cavities |
| L. rhamnosus, | dentine | |
| L. gasseri, L. casei, | | |
| L. salivarius, | | |
| L. plantarum | | |
| Streptococcus gordonii | Accessory pathogens that support | Gum, |
| | and increase the pathogenic nature | Cavities |
| | of keystone pathogens. | |
| Streptococcus | Accessory pathogen similar to | Gum |
| parasanguinis | S. gordonii | |
| Streptococcus pyogenes | Main causative agent for strep | Strep throat, |
| | throat. It also can cause other | Systemic |
| | systemic diseases such as | |
| | necrotizing fasciitis and | |
| | streptococcal toxic shock | |
| | syndrome. | |
| Actinomyces naeslundii | Newly recognized periodontitis | Gum, |
| | oral bacteria. | systemic |
| Prevotella denticola | Newly recogonized periodontitis | Gum |
| | oral bacteria. | |
| Campylobacter | Newly recognized periodontitis | Gum |
| sputorum | oral bacteria. | |
| Saccharibacteria | Bacterial numbers increased in | Gum |
| HMT-356 | periodontal inflammation in many | |
| | studies. Part of the periodontal | |
| | red complex. | |
| Selenomonas sputigena | Associated with subgingival and | Gum |
| | supragingival chronic and | |
| | aggressive periodontitis. | |
| Corynebacterium durum | Commonly found in the human | Systemic |
| | oral cavity and known to cause | |
| | throat, respiratory infections. | |
| Corynebacterium | Commonly found in the human | Systemic |
| matruchotii | oral cavity and known to cause | |
| | throat, respiratory infections | |
| Leptotrichia HMT-498, | Typically cause oral and systemic | Gum, |
| HMT-221, HMT-417, | diseases through opportunistic | Systemic |
| L. wadei, L. buccalis, | infection. | |
| L. trevisanii | | |
| Veillonella atypica, | Commonly found in oral cavity | Gum, |
| V. parvula, dispar | and can cause bacteremis. | systemic |
| | Infrequently isolated from human | |
| | diseases | |
| Haemophilius | Commonly detected and isolated | Systemic |
| influenzae | from the human oral cavity. It | |
| | causes pneumonia, meningitis | |
| | and bacteremia. | |
| Prevotella nigrescens | Increases in children with severe | Cavities |
| | early childhood caries | |
| Solobacterium moorei | Halitosis enriched tongue dorsum | Halitosis |
| | bacteria | |
| Atopobium parvulum | Halitosis enriched tongue dorsum | Halitosis |
| | bacteria | |
| Eubacterium sulci | Halitosis enriched tongue dorsum | Halitosis |
| | bacteria | |
| Filifactor alocis | New merging periodontal | Gum |
| | pathogen that plays significant | |
| | role. | |
| Scardovia wiggsiae | Acid producing biofilm bacteria | Cavities |
| | that is associated with severe | |
| | early childhood caries (ECC). | |

### Biological Samples

In certain aspects, any biological sample that comprises nucleic acid (e.g., genomic DNA or RNA) from a subject is suitable for use in the methods of the present disclosure. In certain embodiments, the biological sample may be saliva.

### Kits

Kits are also contemplated. In certain embodiments, the kits may be used for methods of detecting oral bacteria. In certain embodiments, a kit may comprise any of the CRISPR-nuclease detection systems described herein. In certain embodiments, the kit may comprise one or more components selected from the group consisting of one or more crRNAs provided herein (or a nucleic acid encoding the one or more crRNAs provided herein), one or more RNA-guided nucleases described herein (e.g., Cas13a protein) (or a nucleic acid encoding the one or more RNA-guided nucleases described herein), one or more reporters, one or more primers (forward primer, reverse primer), replication protein A (RPA), RNAse inhibitor, ribonucleoside tri-phosphate (rNTP) mix, T7 RNA Polymerase, ethylene glycol-bis(β-aminoethyl ether)-N,N,N′,N′-tetraacetic acid (EGTA), and dithiothreitol (DTT). In certain embodiments, the kit may comprise one or more components listed in Table 3. For example, the kit may comprise one or more components comprising a forward primer, a revers primer, rehydration buffer, Cas13 protein, one or more crRNAs, reporter, RNAse inhibitor, rNTP mix, T7 RNA polymerase, MgCl₂, MgOAc, water, and RPA pellets.

In certain embodiments, the kit may comprise a laminar flow strip. In certain embodiments, the components of the kit may be lyophilized on the laminar flow strip. In certain embodiments, the kit may be a rapid-detection system.

### Methods of Treating/Preventing a Disease

In certain aspects, provided are methods for treating and/or preventing a disease in a subject in need thereof. In certain embodiments the methods of detection may be used to detect one or more oral bacterium. In certain embodiments, the oral bacterium may cause an oral disease. In certain embodiments, the oral disease may be a gum disease (e.g., without limitation, periodontitis, gingivitis), systemic disease, halitosis, cavities, sensitive and loose teeth, cancer, strep throat, oro-dental trauma, noma, ulcers, sores, acute necrotizing ulcerative gingivitis, root caries, or a combination thereof.

In certain embodiments, the methods for treating and/or preventing an oral disease caused by an oral bacterium in a subject in need thereof may comprise (a) contacting a biological sample with a CRISPR-nuclease detection system described herein, (b) detecting the presence of the one or more bacteria in the biological sample; and (c) treating and/or preventing the disease.

A person of ordinary skill in the art will be aware of the various treatments depending on the oral disease. For example, in certain embodiments, treatment of an oral disease may include one or more selected from the group consisting of administering antibiotics to the subject, frequent cleaning and removal of plaque, providing the subject with a filling, providing the subject with a crown, providing root canal therapy to the subject, extracting one or more teeth from the subject's mouth, scaling and root planning, improvement of oral hygiene, gum grafts, or flap surgery.

A person of ordinary skill in the art will be aware of the various preventative measures to provide to the subject depending on the oral disease. For example, in certain embodiments, prevention of an oral disease may include practicing good oral hygiene, providing a balanced diet, limiting sugar and beverages, regular dental check-ups, use of a specific toothbrush or toothpaste that prevent caries or periodontitis bacterial growth.

### Methods of Detecting the Presence of Bacteria

In certain aspects, provided herein are methods for detecting the presence of one or more oral bacteria in a biological sample. In certain embodiments, the methods comprise (a) contacting the biological sample with a CRISPR-nuclease detection system described herein. In certain embodiments, the methods further comprise (b) detecting the presence of the one or more bacteria in the biological sample. In certain embodiments, the CRISPR-nuclease detection system comprises a reporter comprising a molecule that releases a signal upon cleavage of the reporter by the RNA-guided nuclease.

### Methods of Bacterial Lysis

In certain aspects, provided herein are methods for lysing bacterium. In certain embodiments, the methods of lysing bacterium comprise adding EGTA and DTT to a sample. In certain embodiments, the sample may be a saliva sample. In certain embodiments, the sample may be heated for about 1 minute to about 60 minutes, about 10 minutes to about 20 minutes, or a range defined by any of the two preceding values. In certain embodiments, the sample may be heated for about 15 minutes. In certain embodiments, the sample may be heated for about 1, 2, 3, 4, 5, 6, 7, 8, 9, 10, 11, 12, 13, 14, 15, 20, 25, 30, 35, 40, 45, 50, 55, 60 minutes. In certain embodiments, the sample may be heated at about 90° C. to about 120° C., or a range defined by any of the two preceding values. In certain embodiments, the sample may be heated at about 95° C. In certain embodiments, the sample may be heated at about 95° C. for about 15 minutes. In certain embodiments, the EGTA may be added at a concentration of about 100 mM to about 1000 mM EGTA, about 400 mM to about 600 mM, or a range defined by any of the two preceding values. In certain embodiments, the EGTA may be added at a concentration about 100 mM, 200 mM, 300 mM, 400 mM, 500 mM, 600 mM, 700 mM, 800 mM, about 900 mM, about 1000 mM. In certain embodiments, the EGTA may be added at a concentration of about 500 mM. In certain embodiments, the DTT may be added at a concentration of about 0.01 mM to about 200 mM DTT, about 0.05 mM to about 10 mM DTT, or a range defined by any of the two preceding values. In certain embodiments, the DTT may be added at a concentration of about 0.5 mM, 1 mM, 5 mM, 10 mM, 15 mM, 20 mM, 25 mM, 30 mM, 35 mM, 40 mM, 45 mM, 50 mM, 100 mM, 150 mM, 200 mM DTT. In certain embodiments, the DTT may be added at a concentration of about 1 mM.

### EXAMPLES

The foregoing and the following examples are merely intended to illustrate various embodiments of the present disclosure. The specific modifications discussed above are not to be construed as limitations on the scope of the disclosure. It will be apparent to one skilled in the art that various equivalents, changes, and modifications may be made without departing from the scope of the disclosure, and it is understood that such equivalent embodiments are to be included herein.

### Example 1. Materials and Methods for One-Pot SHERLOCK

### Bacterial Strains and Culture Conditions

Bacterial strains, their sources, and their growth conditions are listed in Table 2. Before each experiment, cells from frozen stocks were recovered at the indicated conditions and in the indicated broth medium and then passaged once a day for two days to ensure homogeneity.

TABLE 2
Bacterial strains and growth conditions.

| Species name and | | Disease | | Oxygen |
|---|---|---|---|---|
| abbreviation | Strain | Association | Medium | condition |
| Staphylococcus aureus | F0253A | Oral and | Brain Heart | 23% O2, 77% N2 |
| (Sa) | | Systemic | Infusion | |
| | | Disease | | |
| Scardovia wiggsiae | F0424 | Caries | Brain Heart | 0% O2, 5% CO2, |
| (Sw) | | | Infusion | 95% N2 |
| Streptococcus mutans | F0577 | Caries | Brain Heart | 23% O2, 77% N2 |
| (Sw) | | | Infusion | |
| Porphyromonas | ATCC33277 | Periodontitis | Brain Heart | 0% O2, 5% CO2, |
| gingivalis (Pg) | | | Infusion | 95% N2 |
| Klebsiella pneumonia | Fpn6806 | Oral and | Brain Heart | 0% O2, 5% CO2, |
| (Kp) | | Systemic | Infusion | 95% N2 |
| | | Disease | | |
| Aggregatibacter | AA075 | Periodontitis | Brain Heart | 23% O2, 77% N2 |
| actinomycetemcomitans | | | Infusion | |
| (Aa) | | | | |
| Acinetobacter | 19606 | Oral and | Brain Heart | 23% O2, 77% N2 |
| baumannii (Ab) | | Systemic | Infusion | |
| | | Disease | | |
| Escherichia coli (Ec) | DH52 | Oral and | LB | 23% O2, 77% N2 |
| | | Systemic | | |
| | | Disease | | |
| Fusobacterium | ATCC23726 | Oral and | Columbia | 0% O2, 5% CO2, |
| nucleatum (Fn) | | Systemic | | 95% N2 |
| | | Diseases, as | | |
| | | well as | | |
| | | various | | |
| | | human | | |
| | | cancers | | |

### Genomic DNA Isolation

Genomic DNA (gDNA) was isolated using the MasterPure gram-positive DNA purification kit (Biosearch, #MC85200) according to the manufacturer's protocol with the addition of a bead beating step as follows: 150 μL of PBS bacteria was briefly mixed with 150 μL of Tris-EDTA (TE) buffer and transferred to a stock tube containing glass beads (Sigma, #G8772). Lysozyme was added to the bacterial mix at a final concentration of 2 mg/mL, and incubated for 1 hour at 37° C. Subsequently, the cells were disrupted by a bead beater for 3×30 seconds at 6 m/s with 1-minute pause intervals. Following this step, the gDNA was isolated according to the manufacturer's protocol. All other synthetic DNA was ordered from Integrated DNA Technologies (IDT).

### One-pot SHERLOCK Reaction (Cas13-RPA)

SHERLOCK detection of target DNA was performed in a one-pot reaction, according to a previously described protocol (Kellner 2019), which is incorporated by reference herein), in which Cas13, RPA reagents, reporter, primers, crRNA, and several adjuvant reagents are simultaneously combined together. Reagents were added in the specific order and at the volumes listed in Table 3. Cas13 reagents were repurposed from the SHERLOCK CRISPR SARS-CoV-2 kit; RPA reagents from the TwistAmp Basic Kit (TwistDx, #INTABAS) were used. Briefly, Cas13-RPA mix (16 uL/reaction) was prepared according to Table 3, then pulse mixed for 3 seconds. A Spectra Max iD3 Multi-Mode Microplate reader was preheated to 37° C. Target gDNA was diluted to the desired concentrations and was added at a volume of 4 μL to 20.6 μL of the Cas-RPA mix. The final reaction mixture containing both target gDNA and Cas-RPA mix was homogenized and centrifuged before 20 μL of each reaction was pipetted into a 384-well optical plate (Roche, #5102430001). The fluorescence reading was taken at 485 nm/528 nm using kinetic reads with 5-minute intervals for 60 minutes.

TABLE 3
Reagents used for One-pot SHERLOCK Reaction.

| Components | Vol/reaction (μL) |
|---|---|
| Forward primer | 1 |
| Reverse primer | 1 |
| Rehydration Buffer | 11.8 |
| Cas13 enzyme (0.5 mg/mL) | 0.32 |
| crRNA (1 μM) | 0.56 |
| Reporter (2 μM) | 1.56 |
| RNAse Inhibitor (40 U/μL) | 0.63 |
| rNTP mix (25 mM) | 1 |
| T7 RNA Polymerase (50 U/μL) | 0.5 |
| MgCl₂ (1 M) | 0.23 |
| MgOAc (Magnesium oxide | 1 |
| antacid complex) (280 mM) | |
| H₂O | 1 |
| RPA Pellets | 0.33 pellets |
| Total volume | 20.6 |

### Example 2. CRISPR RNAs Targeting 16S rRNA Genes

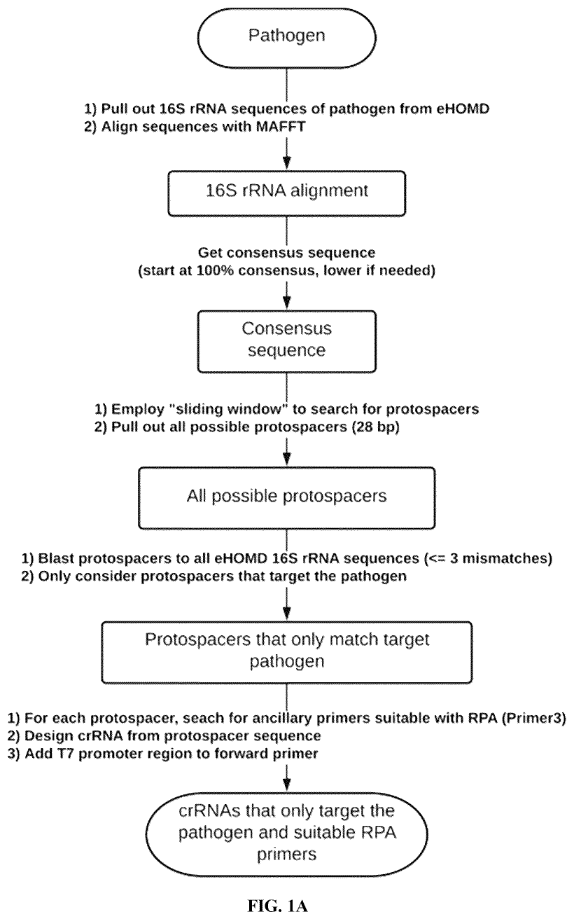

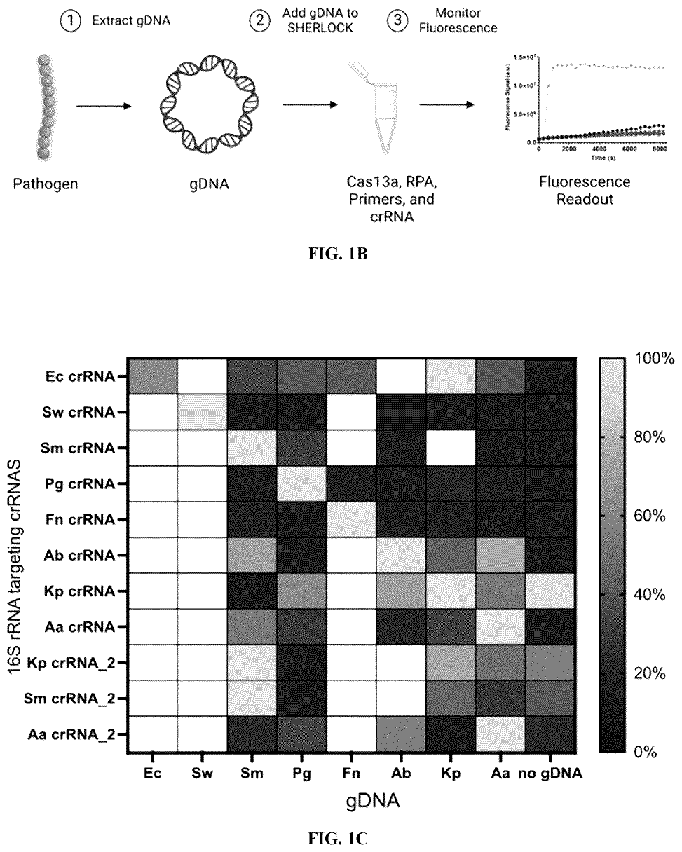

To tailor the SHERLOCK system for the detection of specific targets, two key components were designed: (1) a crRNA that can bind to a cognate 28 base pair region in the target genome (protospacer) and (2) ancillary primers flanking the protospacer that are suitable for RPA. To target specific oral bacteria, the inventors developed a computational pipeline that is capable of outputting ostensible species-specific primers and crRNAs that target the 16S rRNA gene of the bacterial target (FIG. 1A).

Briefly, to design species-specific crRNA and primer pairs targeting 16S rRNA genes suitable for SHERLOCK, a computational pipeline was developed that leverages the expanded Human Oral Microbiome Database (eHOMD) 16S rRNA database of oral bacteria (Escapa, I. F. et al., mSystems 3, e00187-18). Given a particular bacterial species, the computational pipeline first extracted all 16S rRNA gene sequences for the specific species available in eHOMD. The 16S rRNA gene sequences were then aligned using MAFFT (Katoh 2002), and a consensus sequence was determined using the Bio.Align module of Biopython (starting at 100% consensus) (Cock 2009). All possible continuous 28 bp protospacers (no gaps or ambiguities within the protospacer were tolerated) were then extracted. Each protospacer was then BLASTed against the entire eHOMD database using the specific parameters: “—perc_identity 80—word_size 7—gapopen 10—gapextend 2—penalty—1—max_target_seqs 1000”. Only candidate protospacers that contain at least three mismatches to all non-target 16S-RNA gene sequences were retained.

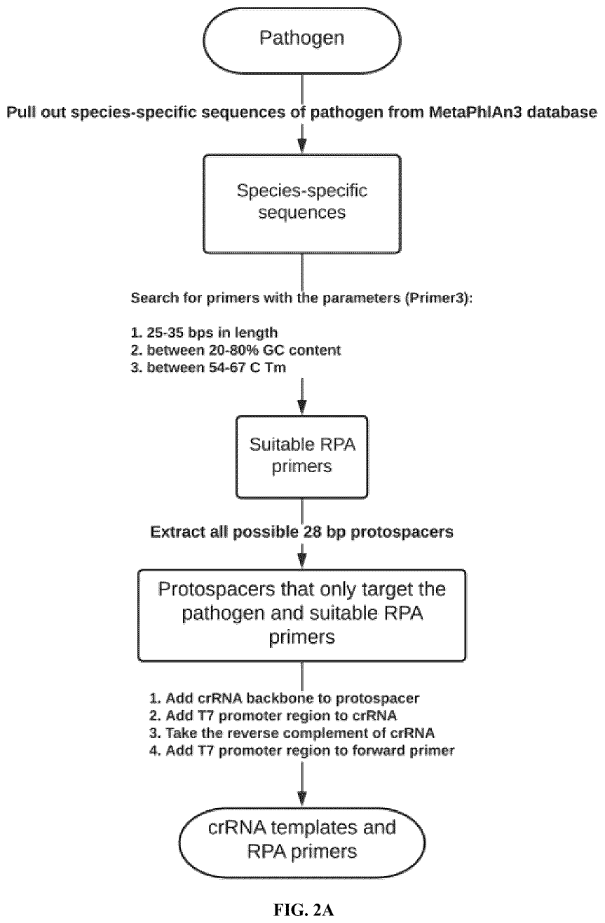

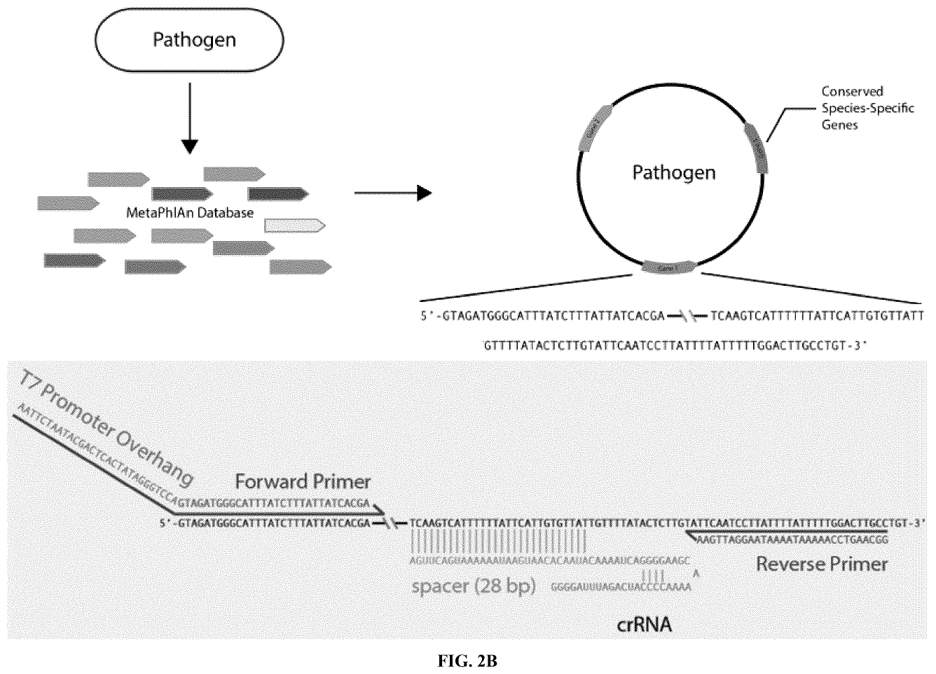

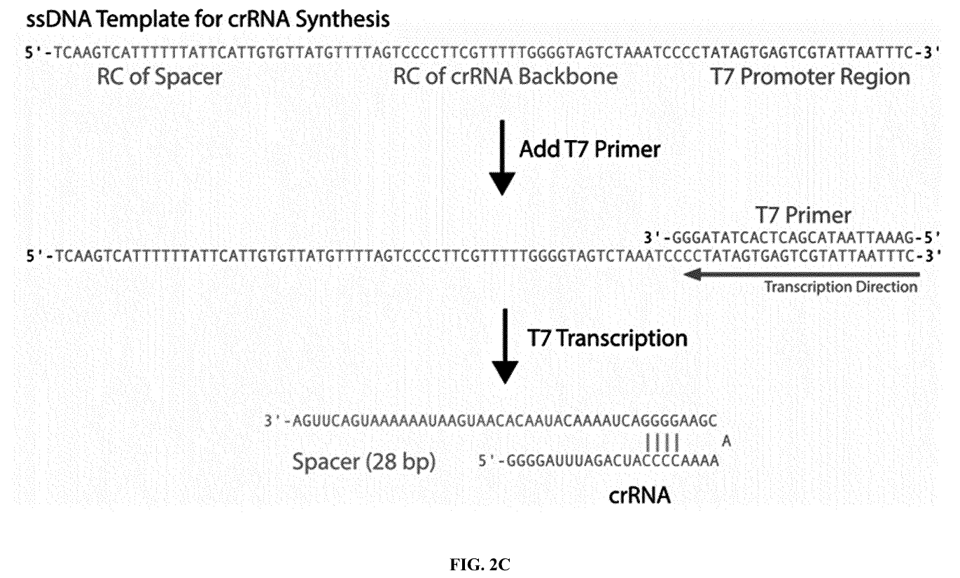

Primer3 was then used on the consensus sequence to design ancillary primers suitable for RPA that flank the protospacer (no gaps or ambiguities present in the consensus sequence are tolerated in the primer design) (Untergasser 2012); Kellner 2019). A T7 promoter (5′-AATTCTAATACGACTCACTATAGGGTCCA-3′ (SEQ ID NO:1)) was then added to the forward primer while the reverse primer was not changed for RPA primer design (see FIG. 2B for an example).

Species-specific crRNAs include a crRNA backbone compatible with LwaCas13a (5′-GGGGAUUUAGACUACCCCAAAAACGAAGGGGACUAAAAC-3′ (SEQ ID NO:2)) added to the spacer (the reverse complement of the predicted protospacer) to form a 67 bp crRNA (see FIG. 2B for an example). If no suitable crRNAs with flanking primer pairs were obtained, the consensus percentage was lowered, and the pipeline was run again.

After putative species-specific crRNAs were predicted, crRNAs were designed using a ssDNA template containing a T7 promoter sequence (5′-TATAGTGAGTCGTATTAATTTC-3′) (SEQ ID NO:124) added to the 3′ end of the reverse complement of the crRNA. All ssDNA templates were ordered from Integrated DNA Technologies (IDT). To translate the ssDNA templates into crRNA, ssDNA templates were annealed with a short T7 primer (5′-GAAATTAATACGACTCACTATAGGG-3′) (SEQ ID NO:127) and incubated with T7 RNA polymerase overnight at 30° C. using the HiScribe T7 Quick High Yield RNA Synthesis kit (NEB, #E2050S) (FIG. 2C). The crRNA was then purified with the Monarch RNA clean up kit (NEB, #T204).

Table 4 provides the protospacer sequences of the 16S rRNA targets from the eight bacterial species (E. coli (Ec), S. mutans (Sm), P. gingivalis (Pg), K. pneumoniae (Kp), A. actinomycetemcomitans (Aa), A. baumannii (Ab), S. wiggsiae (Sw), and F. nucleatum (Fn)), the crRNAs and spacer sequences (RNA and DNA), and specificity demonstrated in cross-reactivity assays described below. Table 5 provides the primers synthesized to target the 16S rRNA gene of the eight bacterial species.

TABLE 4
Protospacers, crRNAs, spacers, and specificity.

| | Protospacer | crRNA | Spacer (5′- | Spacer (5′- | |
|---|---|---|---|---|---|
| *Species | (5′-3′) | (5′-3′)(RNA) | 3′)(RNA) | 3′)(DNA) | Specificity |
| Ec | CTTTCAGC | GGGGAUUUAGA | ACUUUAC | ACTTTAC | Non- |
| | GGGGAGG | CUACCCCAAAA | UCCCUUC | TCCCTTC | Specific |
| | AAGGGAGT | ACGAAGGGGAC | CUCCCCG | CTCCCCG | |
| | AAAGT | UAAAACACUUU | CUGAAAG | CTGAAAG | |
| | (SEQ ID | ACUCCCUUCCU | (SEQ ID | (SEQ ID | |
| | NO: 3) | CCCCGCUGAAA | NO: 25) | NO: 36) | |
| | | G (SEQ ID NO: 14) | | | |
| Sw | TTGCGTCT | GGGGAUUUAGA | AAGCAGT | AAGCAGT | Specific |
| | GGTGTGAA | CUACCCCAAAA | AAGCUUU | AAGCTTT | |
| | AGCTTACT | ACGAAGGGGAC | CACACCA | CACACCA | |
| | GCTT (SEQ | UAAAACAAGCA | GACGCAA | GACGCAA | |
| | ID NO: 4) | GUAAGCUUUCA | (SEQ ID | (SEQ ID | |
| | | CACCAGACGCA | NO: 26) | NO: 37) | |
| | | A (SEQ ID NO: 15) | | | |
| Sm_1 | GGCGTAAA | GGGGAUUUAGA | UCCUGAC | TCCTGAC | Non- |
| | GGGAGCG | CUACCCCAAAA | CGCCUGC | CGCCTGC | Specific |
| | CAGGCGGT | ACGAAGGGGAC | GCUCCCU | GCTCCCT | |
| | CAGGA | UAAAACUCCUG | UUACGCC | TTACGCC | |
| | (SEQ ID | ACCGCCUGCGC | (SEQ ID | (SEQ ID | |
| | NO: 5) | UCCCUUUACGC | NO: 27) | NO: 38) | |
| | | C (SEQ ID NO: 16) | | | |
| Sm_2 | AGTGGCGA | GGGGAUUUAGA | GACAGAC | GACAGAC | Non- |
| | AAGCGGCT | CUACCCCAAAA | CAGAGAG | CAGAGAG | Specific |
| | CTCTGGTC | ACGAAGGGGAC | CCGCUUU | CCGCTTT | |
| | TGTC (SEQ | UAAAACGACAG | CGCCACU | CGCCACT | |
| | ID NO: 6) | ACCAGAGAGCC | (SEQ ID | (SEQ ID | |
| | | GCUUUCGCCAC | NO: 28) | NO: 39) | |
| | | U (SEQ ID NO: 17) | | | |
| Pg | GGCAGCTT | GGGGAUUUAGA | AGUGUCA | AGTGTCA | Specific |
| | GCCATACT | CUACCCCAAAA | GUCGCAG | GTCGCAG | |
| | GCGACTGA | ACGAAGGGGAC | UAUGGCA | TATGGCA | |
| | CACT (SEQ | UAAAACAGUGU | AGCUGCC | AGCTGCC | |
| | ID NO: 7) | CAGUCGCAGUA | (SEQ ID | (SEQ ID | |
| | | UGGCAAGCUGC | NO: 29) | NO: 40) | |
| | | C (SEQ ID NO: 18) | | | |
| Fn | CTTTCAGT | GGGGAUUUAGA | CCGUCAU | CCGTCAT | Specific |
| | TGGGAAGA | CUACCCCAAAA | UUUUUUC | TTTTTTCT | |
| | AAAAAATG | ACGAAGGGGAC | UUCCCAA | TCCCAAC | |
| | ACGG (SEQ | UAAAACCCGUC | CUGAAAG | TGAAAG | |
| | ID NO: 8) | AUUUUUUUCUU | (SEQ ID | (SEQ ID | |
| | | CCCAACUGAAA | NO: 30) | NO: 41) | |
| | | G (SEQ ID NO: 19) | | | |
| Ab | ATGGGAGT | GGGGAUUUAGA | AGCUACU | AGCTACT | Non- |
| | TTGTTGCA | CUACCCCAAAA | UCUGGUG | TCTGGTG | Specific |
| | CCAGAAGT | ACGAAGGGGAC | CAACAAA | CAACAAA | |
| | AGCT (SEQ | UAAAACAGCUA | CUCCCAU | CTCCCAT | |
| | ID NO: 9) | CUUCUGGUGCA | (SEQ ID | (SEQ ID | |
| | | ACAAACUCCCA | NO: 31) | NO: 42) | |
| | | U (SEQ ID NO: 20) | | | |
| Kp_1 | GAGATGGA | GGGGAUUUAGA | ACAGUUC | ACAGTTC | Non- |
| | TTGGTGCC | CUACCCCAAAA | CCGAAGG | CCGAAG | Specific |
| | TTCGGGAA | ACGAAGGGGAC | CACCAAU | GCACCAA | |
| | CTGT (SEQ | UAAAACACAGU | CCAUCUC | TCCATCT | |
| | ID NO: 10) | UCCCGAAGGCA | (SEQ ID | C (SEQ ID | |
| | | CCAAUCCAUCU | NO: 32) | NO: 43) | |
| | | C (SEQ ID NO: 21) | | | |
| Kp_2 | CAGGGCTA | GGGGAUUUAGA | AUGCCAU | ATGCCAT | Non- |
| | CACACGTG | CUACCCCAAAA | UGUAGCA | TGTAGCA | Specific |
| | CTACAATG | ACGAAGGGGAC | CGUGUGU | CGTGTGT | |
| | GCAT (SEQ | UAAAACAUGCC | AGCCCUG | AGCCCTG | |
| | ID NO: 11) | AUUGUAGCACG | (SEQ ID | (SEQ ID | |
| | | UGUGUAGCCCU | NO: 33) | NO: 44) | |
| | | G (SEQ ID NO: 22) | | | |
| Aa 1 | TAGGGAGC | GGGGAUUUAGA | CAUGCAG | CATGCAG | Non- |
| | TTTGAGAC | CUACCCCAAAA | CACCUGU | CACCTGT | Specific |
| | AGGTGCTG | ACGAAGGGGAC | CUCAAAG | CTCAAAG | |
| | CATG (SEQ | UAAAACCAUGC | CUCCCUA | CTCCCTA | |
| | ID NO: 12) | AGCACCUGUCU | (SEQ ID | (SEQ ID | |
| | | CAAAGCUCCCU | NO: 34) | NO: 45) | |
| | | A (SEQ ID NO: 23) | | | |
| Aa 2 | GGATGTAC | GGGGAUUUAGA | GCUUUCG | GCTTTCG | Non- |
| | TGACGCTG | CUACCCCAAAA | CACAUCA | CACATCA | Specific |
| | ATGTGCGA | ACGAAGGGGAC | GCGUCAG | GCGTCAG | |
| | AAGC (SEQ | UAAAACGCUUU | UACAUCC | TACATCC | |
| | ID NO: 13) | CGCACAUCAGC | (SEQ ID | (SEQ ID | |
| | | GUCAGUACAUC | NO: 35) | NO: 46) | |
| | | C (SEQ ID NO: 24) | | | |

*Species abbreviation: E. coli (Ec), S. mutans (Sm), P. gingivalis (Pg), K. pneumoniae (Kp), A. actinomycetemcomitans (Aa), A. baumannii (Ab), S. wiggsiae (Sw), and F. nucleatum (Fn).

TABLE 5
Primers for One-pot SHERLOCK Reaction.

| *Species | Forward Primer (5′-3′) | Reverse Primer (5′-3′) |
|---|---|---|
| Ec | AATTCTAATACGACTCACTATAGG | CGGGTAACGTCAATGAGCAAAG |
| | GTCCAGGGAATATTGCAATGGGC | GTATT (SEQ ID NO: 58) |
| | GCAAG (SEQ ID NO: 47) | |
| Sw | AATTCTAATACGACTCACTATAGG | TTACACCGAGAATTCCAGTCTCC |
| | GTCCAGTTGTCCGGATTTATTGG | CCTACTG (SEQ ID NO: 59) |
| | GCGTAAAG (SEQ ID NO: 48) | |
| Sm_1 | AATTCTAATACGACTCACTATAGG | GTTGAGCCATAGCCTTTTACTCC |
| | GTCCAGTAGCTTACCAGAAAGGG | AGA (SEQ ID NO: 60) |
| | ACGGCTAACTAC (SEQ ID NO: 49) | |
| Sm_2 | AATTCTAATACGACTCACTATAGG | GACTACCAGGGTATCTAATCCTG |
| | GTCCAGTGAAATGCGTAGATATA | TTC (SEQ ID NO: 61) |
| | TGGAGGAACAC (SEQ ID NO: 50) | |
| Pg | AATTCTAATACGACTCACTATAGG | CTAGTAATCATCGTTTACTGCGT |
| | GTCCAGTGAAATGCATAGATATC | GGACTAC (SEQ ID NO: 62) |
| | ACGAGGAACTC (SEQ ID NO: 51) | |
| Fn | AATTCTAATACGACTCACTATAGG | CACGTATTTAGCCGTCACTTCTT |
| | GTCCAGAATATTGGACAATGGAC | CTGTTGGT (SEQ ID NO: 63) |
| | CGAGAGTCTGAT (SEQ ID NO: 52) | |
| Ab | AATTCTAATACGACTCACTATAGG | GTGGTAACCGCCCTCTTTGCAG |
| | GTCCAGAATCGCTAGTAATCGCG | TTAG (SEQ ID NO: 64) |
| | GATCAGAATG (SEQ ID NO: 53) | |
| Kp_1 | AATTCTAATACGACTCACTATAGG | GACTTAACCCAACATTTCACAAC |
| | GTCCACTTACCTGGTCTTGACAT | ACGAG (SEQ ID NO: 65) |
| | CCACAGAACTTT (SEQ ID NO: 54) | |
| Kp_2 | AATTCTAATACGACTCACTATAGG | GGACTACGACATACTTTATGAGG |
| | GTCCAGTGATAAACTGGAGGAAG | TCC (SEQ ID NO: 66) |
| | GTGGGGATGAC (SEQ ID NO: 55) | |
| Aa_1 | AATTCTAATACGACTCACTATAGG | GACTTAACCCAACATTTCACAAC |
| | GTCCACTTACCTACTCTTGACATC | ACGAG (SEQ ID NO: 67) |
| | CGAAGAAGAAC (SEQ ID NO: 56) | |
| Aa_2 | AATTCTAATACGACTCACTATAGG | ATCCCCAAATCGACACCGTTTAC |
| | GTCCAGAAATGCGTAGAGATGTG | AG (SEQ ID NO: 68) |
| | GAGGAATAC (SEQ ID NO: 57) | |

*Species abbreviation: E. coli (Ec), S. mutans (Sm), P. gingivalis (Pg), K. pneumoniae (Kp), A. actinomycetemcomitans (Aa), A. baumannii (Ab), S. wiggsiae (Sw), and F. nucleatum (Fn).

The cross-reactivity of the 16S-rRNA gene targeting crRNAs against the genomic DNA (gDNA) of the other bacterial targets was assessed (FIG. 1B). Briefly, in order to test the specificity of the designed crRNAs against gDNA from other oral bacterial pathogens, individual Cas13-RPA reactions were run for each oral pathogen as described above in Example 1 for the “One-pot SHERLOCK Reaction (Cas13-RPA).” The crRNAs from Table 4 and primers from Table 5 were used in the assay. 4 μL of gDNA was added to separate aliquots of 20.6 μL of Cas mix for a final concentration of 1 picomolar of target gDNA. 20 μL of each of these reactions was then transferred to individual wells on 384-well optical plate for fluorescence reading.

While there was successful detection of three oral pathogens, F. nucleatum (Fn), S. wiggsiae (Sw), and P. gingivalis (Pg), at a high specificity, the 16S-rRNA gene-targeting crRNAs were found to consistently display non-specific signal (FIG. 1C). Only 3 of the 11 crRNAs that were designed displayed a specific signal for their target bacterial gDNA (FIG. 1C, Table 4).

As the computational pipeline designed crRNAs with at least three mismatches to other non-target 16S rRNA sequences in the eHOMD database, these data first suggest that the SHERLOCK system may have a higher tolerance to mismatches than previously reported (1 mismatch). Alternatively, perhaps the eHOMD 16S rRNA database is under-sampled and does not encompass enough 16S rRNA diversity among oral bacteria for specific crRNA design. Nevertheless, the 16S rRNA gene may be too conserved across many species for it to be a suitable target for consistent, reliable species-specific detection using SHERLOCK.

### Example 3. High Specificity from CRISPR-RNAs that Target Conserved Species-Specific Genes

As crRNAs targeting the 16S rRNA gene of bacteria exhibited more than expected non-specific signal as shown in Example 2, the inventors designed a new computational pipeline that co-opts a previously compiled database of species-specific genes with conserved nucleotide sequences, Metagenomic Phylogenetic Analysis (MetaPhIAn), to design primer pairs and crRNAs suitable for SHERLOCK (FIGS. 2A-2B).

Briefly, to design crRNA and primer pairs suitable for SHERLOCK that target conserved species-specific genes, a computational pipeline was developed that leverages the MetaPhIAn database, which is composed of species-specific genes with conserved nucleotide sequences (Beghini 2021). First, all genes specific to a given bacterial species were extracted from the MetaPhIAn database (FIG. 2A). Specifies-specific refers to genes that are only present in a single species. Species-specific genes from seven oral pathogens (A. actinomycetemcomitans, A. baumannii, K. pneumoniae, P. gingivalis, S. aureus, S. mutans, and S. wiggsiae) were selected for targeting. The species-specific genes that were selected are set forth below in SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus).

The pipeline then extracted all possible candidate 28 bp protospacers from each gene. For each protospacer, Primer3 was used to design ancillary primers suitable for RPA that flank the protospacer. As with the 16S rRNA targeting crRNA pipeline described in Example 2, a T7 promoter sequence was added to the forward primer and a Cas13a crRNA backbone was added to the spacer. crRNA was then synthesized using the ssDNA template method as described above.

Table 6 provides the protospacer sequences of the specifies-specific target genes, the crRNA sequences, the spacer sequences of the crRNAs (RNA and DNA), and specificity demonstrated in cross-reactivity assays described below. Table 7 provides the RPA primers synthesized to for each of the bacterial species-specific genes tested.

TABLE 6
Protospacers, crRNAs, spacers, and specificity.

| | Protospacer | crRNA | Spacer (5′- | Spacer (5′- | |
|---|---|---|---|---|---|
| Species* | (5′-3′) | (5′-3′)(RNA) | 3′)(RNA) | 3′)(DNA) | Specificity |
| Sm | TCAAGTCAT | GGGGAUUUAG | AUAACAC | ATAACAC | Specific |
| | TTTTTATTCA | ACUACCCCAAA | AAUGAAU | AATGAAT | |
| | TTGTGTTAT | AACGAAGGGG | AAAAAAU | AAAAAAT | |
| | (SEQ ID | ACUAAAACAUA | GACUUGA | GACTTGA | |
| | NO: 69) | ACACAAUGAAU | (SEQ ID | (SEQ ID | |
| | | AAAAAAUGACU | NO: 83) | NO: 90) | |
| | | UGA (SEQ ID | | | |
| | | NO: 76) | | | |
| Sw 1 | ATATTATTCT | GGGGAUUUAG | AAAGAAA | AAAGAAA | Non- |
| | TATTCTTCTT | ACUACCCCAAA | AAAGAAG | AAAGAAG | specific |
| | TTTTCTTT | AACGAAGGGG | AAUAAGA | AATAAGA | |
| | (SEQ ID | ACUAAAACAAA | AUAAUAU | ATAATAT | |
| | NO: 70) | GAAAAAAGAAG | (SEQ ID | (SEQ ID | |
| | | AAUAAGAAUAA | NO: 84) | NO: 91) | |
| | | UAU (SEQ ID | | | |
| | | NO: 77) | | | |
| Sw 2 | TCATATTATT | GGGGAUUUAG | AGAAAAA | AGAAAAA | Specific |
| | CTTATTCTTC | ACUACCCCAAA | AGAAGAA | AGAAGAA | |
| | TTTTTTCT | AACGAAGGGG | UAAGAAU | TAAGAAT | |
| | (SEQ ID | ACUAAAACAGA | AAUAUGA | AATATGA | |
| | NO: 71) | AAAAAGAAGAA | (SEQ ID | (SEQ ID | |
| | | UAAGAAUAAUA | NO: 85) | NO: 92) | |
| | | UGA (SEQ ID | | | |
| | | NO: 78) | | | |
| Aa | CATTACTTC | GGGGAUUUAG | AUAACGA | ATAACGA | Specific |
| | ATTATACTA | ACUACCCCAAA | GGCUAG | GGCTAGT | |
| | GCCTCGTTA | AACGAAGGGG | UAUAAUG | ATAATGA | |
| | T (SEQ ID | ACUAAAACAUA | AAGUAAU | AGTAATG | |
| | NO: 72) | ACGAGGCUAG | G (SEQ ID | (SEQ ID | |
| | | UAUAAUGAAG | NO: 86) | NO: 93) | |
| | | UAAUG (SEQ ID | | | |
| | | NO: 79) | | | |
| Pg | TGCTGTATC | GGGGAUUUAG | AAGUGGA | AAGTGGA | Specific |
| | TTATCTCCTT | ACUACCCCAAA | CAAAGGA | CAAAGGA | |
| | TGTCCACTT | AACGAAGGGG | GAUAAGA | GATAAGA | |
| | (SEQ ID | ACUAAAACAAG | UACAGCA | TACAGCA | |
| | NO: 73) | UGGACAAAGG | (SEQ ID | (SEQ ID | |
| | | AGAUAAGAUAC | NO: 87) | NO: 94) | |
| | | AGCA (SEQ ID | | | |
| | | NO: 80) | | | |
| Ab | GGTCTGTTT | GGGGAUUUAG | AAAUUAU | AAATTAT | Specific |
| | GCTATATTTT | ACUACCCCAAA | UGAAAAU | TGAAAAT | |
| | CAATAATTT | AACGAAGGGG | AUAGCAA | ATAGCAA | |
| | (SEQ ID | ACUAAAACAAA | ACAGACC | ACAGACC | |
| | NO: 74) | UUAUUGAAAAU | (SEQ ID | (SEQ ID | |
| | | AUAGCAAACAG | NO: 88) | NO: 95) | |
| | | ACC (SEQ ID | | | |
| | | NO: 81) | | | |
| Kp | CAGTGATGG | GGGGAUUUAG | AUUUUUU | ATTTTTTA | Specific |
| | CAGTGTTTA | ACUACCCCAAA | AAAUAAA | AATAAAC | |
| | TTTAAAAAAT | AACGAAGGGG | CACUGCC | ACTGCCA | |
| | (SEQ ID | ACUAAAACAUU | AUCACUG | TCACTG | |
| | NO: 75) | UUUUAAAUAAA | (SEQ ID | (SEQ ID | |
| | | CACUGCCAUC | NO: 89) | NO: 96) | |
| | | ACUG (SEQ ID | | | |
| | | NO: 82) | | | |
| Sa | AACTACATA | GGGGAUUUAG | AAAAAUA | AAAAATA | Specific |
| | TGTTTTTCTG | ACUACCCCAAA | AGCAGAA | AGCAGAA | |
| | CTTATTTTT | AACGAAGGGG | AAACAUA | AAACATA | |
| | (SEQ ID | ACUAAAACAAA | UGUAGU | TGTAGTT | |
| | NO: 120) | AAUAAGCAGAA | U (SEQ ID | (SEQ ID | |
| | | AAACAUAUGUA | NO: 122) | NO: 123) | |
| | | GUU (SEQ ID | | | |
| | | NO: 121) | | | |

*Species abbreviations = A. actinomycetemcomitans (Aa), A. baumannii (Ab), K. pneumoniae (Kp), P. gingivalis (Pg), S. aureus (Sa), S. mutans (Sm), and S. wiggsiae (Sw)

TABLE 7
Primers for One-pot SHERLOCK Reaction.

| Species* | Forward Primer (5′-3′) | Reverse Primer (5′-3′) |
|---|---|---|
| Sm | AATTCTAATACGACTCACTATAG | GGCAAGTCCAAAAATAAAATAAG |
| | GGTCCAGTAGATGGGCATTTATC | GATTGAA (SEQ ID NO: 105) |
| | TTTATTATCACGA (SEQ ID NO: 97) | |
| Sw 1 | AATTCTAATACGACTCACTATAG | ATGAAGAGGAATAAGGATGAAG |
| | GGTCCATCCCTTATTCTTTCTTAT | ATGAGATG (SEQ ID NO: 106) |
| | TTCCTCTGTTTT (SEQ ID NO: 98) | |
| Sw 2 | AATTCTAATACGACTCACTATAG | GAAGAGGAATAAGGATGAAGAT |
| | GGTCCATTCCCTTATTCTTTCTTA | GAGATGAG (SEQ ID NO: 107) |
| | TTTCCTCTGTTT (SEQ ID NO: 99) | |
| Aa | AATTCTAATACGACTCACTATAG | TGCAATATGAATAATAGAGGTAT |
| | GGTCCACCCTTTGTTAAAAACGA | CAGGAGA (SEQ ID NO: 108) |
| | TAGCAGAAAAATA (SEQ ID | |
| | NO: 100) | |
| Pg | AATTCTAATACGACTCACTATAG | AATCACTTTTTCACTCGTATAGG |
| | GGTCCAGATTTTCTTATCGGATT | TCAGTTG (SEQ ID NO: 109) |
| | TCACAAATGACTG (SEQ ID | |
| | NO: 101) | |
| Ab | AATTCTAATACGACTCACTATAG | GCAGTACAAATGAAAATTGAACC |
| | GGTCCATACAATAATGCTAGATT | AGATTTA (SEQ ID NO: 110) |
| | TATCGCACTAGTG (SEQ ID | |
| | NO: 102) | |
| Kp | AATTCTAATACGACTCACTATAG | GATTTTATAAATTGATGCCATCT |
| | GGTCCAGATACGATGATCTTCAG | GCTGATT (SEQ ID NO: 111) |
| | GCGTTATACAATG (SEQ ID | |
| | NO: 103) | |
| Sa | AATTCTAATACGACTCACTATAG | AAGCGACAATCTAACATTAAAGA |
| | GGTCCAGAAACCTTTGCTATAAT | AGTGATA (SEQ ID NO: 112) |
| | ACCTATCGTCTTT (SEQ ID | |
| | NO: 104) | |

*Species abbreviations = A. actinomycetemcomitans (Aa), A. baumannii (Ab), K. pneumoniae (Kp), P. gingivalis (Pg), S. aureus (Sa), S. mutans (Sm), and S. wiggsiae (Sw)

The following are the sequences of the conserved species-specific genes used in Example 3 to generate the crRNAs in Table 6 and the RPA primers in Table 7:

S. mutans (Sm), Gene ID: 1309_Q93ES7_mreD, Gene Sequence:
(SEQ ID NO: 113)
ATGTCTATCTTTAAAAATAAATTATTTGTCATTTTATTTGCCTTTCTTATGTTACTTGT
AGATGGGCATTTATCTTTATTATCACGAATCTTATTTCAAAATCAGTTTATTGTGTCA
AGTCATTTTTTATTCATTGTGTTATTGTTTTATACTCTTGTATTCAATCCTTATTTTATT
TTTGGACTTGCCTGTGTTTTAGGAATTATTTATGATTTTTATTATTTAGGGGTATATA
ATTTGGGAATTGCAACTATGTTGTATCCTTTAACAATTGTTATTATGTTTAAATTATG
GAAACATATTCCAAATGGTCCTGTTCAACGCTTTCTAGTTTTTTTTATCCTAATCTTT
TTTCTTGATTTTGCTAGTATTGGTATGGCCTATTTATATCAATTGACAGCTTATCCTT
TAAATGATTTTATCACTTATAATTTAGCACCTTCCTTAATTTTTAATATACTGGCTTTT
CTCTTCTTTCAAAAACTTTTAGAAAGGATTTACCTATGA
S. wiggsiae (Sw) 1 and 2, Gene ID:
230143_J0LL06_HMPREF9156_00967, Gene Sequence:
(SEQ ID NO: 114)
ATGATAGTTAAGGGGGGGGAAATAATCCCGCCCCTTAACTATTTCCCTTATTCTTTC
TTATTTCCTCTGTTTTCATATTATTCTTATTCTTCTTTTTTCTTTTTACCCTTTCGCACT
CGCCCTCTTCACCTGCGTTCTCCTTCTTCCTTCCCTCATCTCATCTTCATCCTTATT
CCTCTTCATCCTCGCTCCGGTCGTCATCCCGCTTCACCTTCCAATCTCACTCTGCC
CTTCCTTACACCCTGCCTTTTCTTACTCTCCAATCTCATCTCACCTCCTTATTTTTCG
ATCTCACCCCGTTCTTCCTTACTCTCCGATTTCATCTCACTTCTTTACTTTCCGACCT
TTTATCCACATACATATGTAGCCCCACTTCCGACCTCACTCCATCTCTCCCTACCTT
CTGACCTCACTCCATACCCTCCTTACTTTCCGATCGTACTCCACCATCTCCTTACTO
TCCAATCTCACCCCATCCCCTCACCCAACTCGAAGATACGGATCCTCCTCCGACG
GCCTCCTCCCACATCCGTACAGCTGAAACTATAA
A. actinomycetemcomitans (Aa), Gene ID:
714_A0A142G015_D17P2_0309860, Gene Sequence:
(SEQ ID NO: 115)
ATGAAAGCATTACTCCGAAAAATCCGTTTAGCCTTAGGCAAAATGTTGCTAGACAAA
AATGTTCAGGGGCAGGCTTTGCCGGCAAATCCGAAAATTATTGTGTTACAACAAGA
CGGAAAAATCGGAGATTATATTGTCAGTTCATTTATTTTCAGAGAATTAAAACGACA
TAACCCTAAAATGCAGGTTGATGTTGTATGTTCACCTAAAAATGTCAATTTATTTGAA
CAAAATCCATCTATTGATCATTGTTTTATTTTAAATAGAAAAGAACATTGCGCTTACA
GCAGAATGGGAAAACAACTTTCACATGAACATTATGATGTATTAATTAATTTACCGG
TATTATTACGGAATCGTGATTTGTGGCTAACCCGATTAATTCATGCAAAAAATAATA
TCGGCTATAAAAAACAAAACTACAAACTATTTAATTTGAATGTTACTCAAGATCAATT
ACATTTTTCCAAAGTTTATGCAGAAGCCATTAAATTATGTGGTGTAAAAGATATTAAT
CTTGAATATGACATTCCAAATCACAGTGATAAAAAAGAAGATATTGCAAATTTCATA
CAAAAAAATCATCTTGTCGATTGCATCGCTATAAACTTTTTTGGTGCAGCCGGCACC
AGAAAATTTACGGAACAAAACATTTATCGGTTTATGGAAAAATTTAAAGCCGAAAAT
AAAAAAGCGCTGTTACTAACTTACCCCGAAGTAACCCCTTTGTTAAAAACGATAGCA
GAAAAATATACCAATGCTTTTATCTATGAAAATACTGAAAATATCTTTGATACGATAA
CATTACTTCATTATACTAGCCTCGTTATTTCTCCTGATACCTCTATTATTCATATTGC
AGCAGGATTAAATAAAAAAATTATTGGATTTTATAAGCTCGCTGATAAAGAAAACTTT
ACGCATTGGAATCCAAACTGTAAAAATAAAACATATATCCTTAATTTTATTGAAAATG
TCAATGAAATCTCTCCCGATGAAATTAAATCAGAATGGTTAAAATAA
P. gingivalis (Pg), Gene ID: 837_B2RJX1_CBG53_02860, Gene
Sequence:
(SEQ ID NO: 116)
GTGGCAAAATCCGATAGAACGTTTTTAATGCCGGTTACGATATTGATCGCCCTCGT
GTGTAGGATGCTGTCCTACGCATGGGGGCTTTCTGCTGATATTTCTCTTCAAGAGT
GTAGTCTTCCCCTCTTCGGCACGATCCCTTTCTTATGGCAAACCGTTATCTCGTTTG
CCATTGGCTTGGTTGCATCTTTTATTGCGGTTCGCTTCAGTGCTTTCTATCTTCTGC
TTCATGAAGGTGGATTCCGGCCATTTGCTTTCTTGATGATTCTTTTGCTGAACACCC
ATCAGGTTTTCTTCCCCATGCAGCCATACTCGCTGTCGATACTGCTGCTGATAGTA
CTCTTTTTCTGCCTTTTTGGCACTTATGGCCGTAATAATATCCCGCCCAAGATGCTT
AACGTAGGCTTTTGTGTCGGGCTTTCGGCTGTGTTGTGGTCTCCTTCGCTGCTTCT
TGCACCGTTTGTGCTCATACAGTTTTATCTGATGAAGAGTTTGTCTTTCAAAAACTT
GATTGCATTTTTCTTCGGACTGTTGCTTCCTCTGTGGTGCTGTCTTCCTTTATTGGT
ATTGGCCGGACAGGAGCAGTTTGTTATCGACAATATGACCCTTTTGACTCGGTGGG
GATTTTCTTATCGGATTTCACAAATGACTGCGTGGAAGGGGCTGTATCCGGCTCTA
CTGTCAGTGCTGTATCTTATCTCCTTTGTCCACTTGCAACTGACCTATACGAGTGAA
AAAGTGATTGCACGCATTTACTTCTTCAGCCTTTTGTGTGCCGGTTGTTATATGCTG
CTACTCTCTGCCGTGATGCCTGCTGCTTCCGAAGGATTCGTATATCTGGCTACCTT
CCCTGTGTCGGTACATGCAGCTCGTTTCTTGAATTCTCTGAATCGTCGTGTTGCCA
ATATCTTGATTCCTTTTACGTTTTTGGTTTTCTTGTCCTCTTTTTTGCTGTCTTCTTTT
TTCCTTTAG
A. baumannii (Ab), Gene ID: 470_A0A0E1FMW9_BWI80_12865, Gene
Sequence:
(SEQ ID NO: 117)
ATGGCTAAACGCTACTTACCCTTCTACAATAATGCTAGATTTATCGCACTAGTGTTA
GTCGGTCTGTTTGCTATATTTTCAATAATTTTTAAATATTTAGAGTTAAATATTACAAT
AAATCTGGTTCAATTTTCATTTGTACTGCTTCTCCCTTTAAGTCAAATTTATTTGGCT
TATAAAGGTATGCTCGATGCATTAAAGCTTGATGGTTTAAATCAGTCAGAACGAGAT
AGGTTGACTTCAACTGTGGACATAAGAAGTAAGTCATCTTTATATGTGGCTATGCTT
TTTATTATTCTTGTTTTTAGTATGTATATACTTAATTTATTAGGCTTACTTTCAGCTAA
GCATCTTTTAGCTCTAATACTTTCTGTTGGACTCACCTCAATTTTTAGCTTCTTCTTA
GCTTGGTCTGACTTAAGAGAAATCTCTTTGCTTGAAAAAACATTAAAAGATCGCAAA
GAATCAAGAGAGGCAAAAGCAAAAGTATTGAGCAATAAGTAA
K. pneumoniae (Kp), Gene ID:
573_A0A1D3KMF9_SAMEA2273765_03871, Gene Sequence:
(SEQ ID NO: 118)
GTGAAACAGACCCATTATTTCACCGTGAATTTTACCGGCTTCACCACTGCCGCCTC
GGAAGAGCAAAGCTATTTACGCCTCATTGCAGGCGAGCATGCCTTTTATACCGATA
AACGCCATTTTAAAGACCCCTCGCTTTTTGACCGTCTGAGGCTCGGCCAGCCGCTA
CATATCGGCACTTGCCGCCTAAAGGATGGCAGTTACTGGATCCACTGGTTAAGCG
ATGGCCACATTTTGCTCGAACCTTCCCGGCAGCCGCTGAGTATTAAAAAGCTTCTT
CGACCTGTCGGCTTGGTCTTTTTTGTTTGCTTGATCATTCTCGCTTGTGCTCCATGG
TTGGATATTTTTTTATTATTATGTTTTATCACAATGTTGGTGAATCCCTTCGGCATCT
TCTCTTTTTGCTCTTGCAATTTCTCACTCAGGTTACGTGTTCTCAATGCGAAAATGC
AGCGAACGAAGCAAGGCGATATTTCATTCTGTCAGCGTCTGGAAATTCTACCCGCT
ACTTCTGAAAGGCATCCACCATCGGCCAACAACGAGATAGCGCTGCCAAAAGAATT
TGCGCTTGAAGAAGGCGTGATTCTGAATAGTTATTATCAAATATGGATCGCTACGA
GGGTAAGGCCTTATTTTATCAAAGAAGGCGCCTGTTTTCAGCTGGCGACTCGTCAC
TCGTTTTTTTCAGCGCGCTTGACCTGGCTGAGCAATGCCCTGAATAATCGTTGTCG
CCCGCCATTTCTTGCCGATGGGGACTGGGTTATCGCCGCACGCCGGCATGGATAC
GATGATCTTCAGGCGTTATACAATGTCAGTGATGGCAGTGTTTATTTAAAAAATAGC
CCCTTTCATCCCGGCAATCAGCAGATGGCATCAATTTATAAAATCGCGTACGGCCT
CGTACTCATAGTGATGTTTTTTACGCTCACCATCAAACTGCACGACCCCATGCTTAA
TCTGGGCAGTTGGCCATTTTATCGTGAAATGCTTACCAGGCTGACAGGCACCCTGC
TCCCGATTAACCTGCTGTTAATAGTGATGGAGTTTGTCCGTATGATTGCCCATCCC
CTGTCACGTCGGGTGGCCGGTAGAGTGAAGGTACATCAGGCCATATTACGTTATG
CCAGACGTCAGGGCGCCAGAATGACGCCATGGGAACTGAAGTAA
S. aureus (Sa), Gene ID: 1280_A0A386C2Q6_
B8A11_00750, Gene Sequence:
(SEQ ID NO: 119)
TTGGTAGTATTGTTATCTTATAGTTGCAGTTGTATCTTTAGTTTTCTATATCAATTTAT
ATCTATAGAAGAAACGTCATTTGATTATTTACATAGAAGATCGAAATGTGATTATTGT
AATTCATCACTCAAATGGTATGAATTAATGCCGATTATTAGTTTTTTATTATTAAAAG
GGCGATGTCGAAACTGTCGAAAGCGTATTTCCCTAACACATTTCTTAGGGGAAACC
TTTGCTATAATACCTATCGTCTTTATTAAGTATGATTTCACATACGTAAATGCTACGC
TATTTATAACTACATATGTTTTTCTGCTTATTTTTACTATGACCGATATCACTTCTTTA
ATGTTAGATTGTCGCTTAATTATAATTTATTGTATCGTTTCTCTCTCGTTAAGTATGA
TTTATCCAGTAGCTTTTATCATTATTAGTATGACCACGCATATATTCTACTTTTTATTT
CGGGCATATATTGGTTATGGTGACGTTTTACTAATATCTGCACTTTCTTTGTTTTTCC
CTCTCCAATTCACTATTTATGTCATTTTATTTACATTTGTCATTGCTGGTTTAGTTGC
TTTAATTACCATGATATTTAAGCCGATTAAACTATTACCCCTTGTTCCATTTATATTTA
TTTCATTTTTTATCAATTCACTTTTTTATAATGATATCCATCAATTTTTAGGAGGCGTA
TATTTTTGA

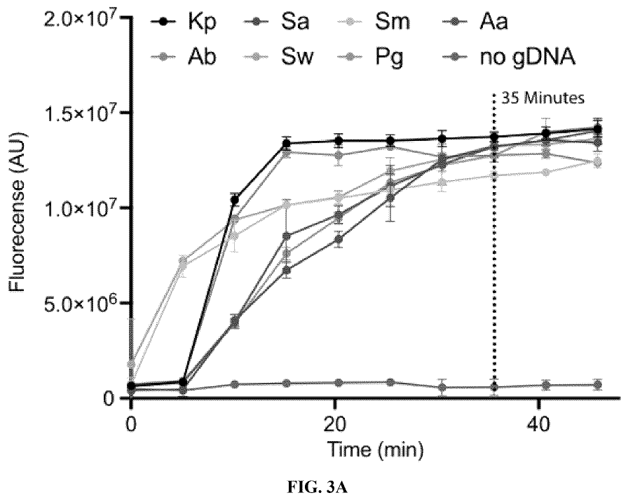

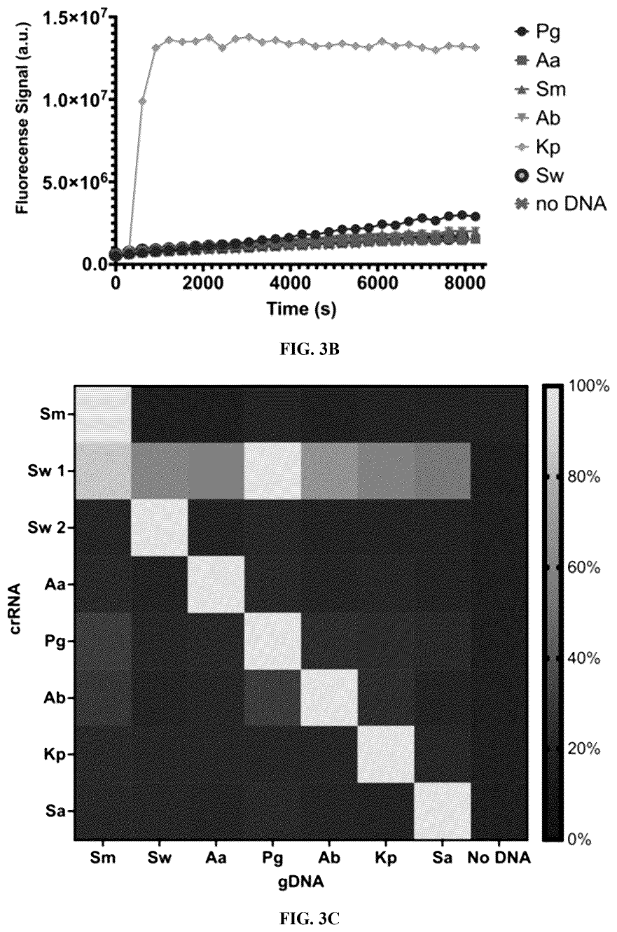

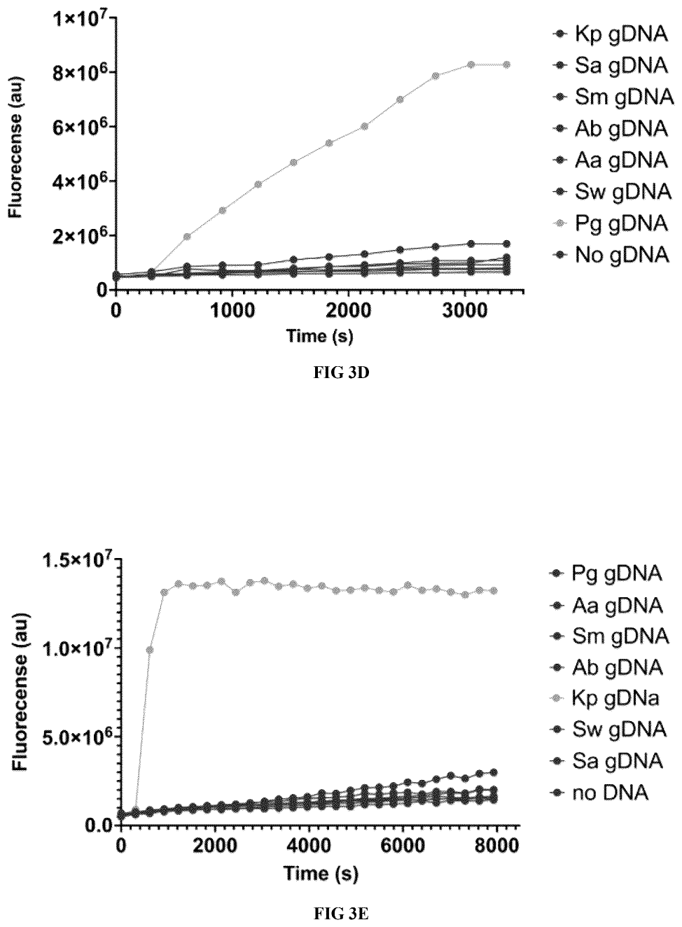

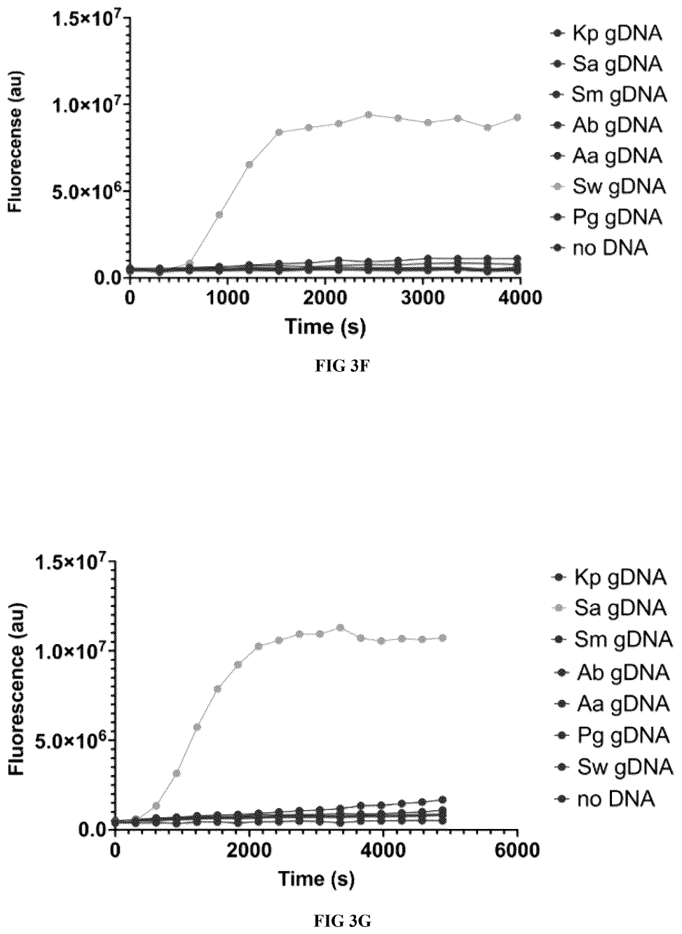

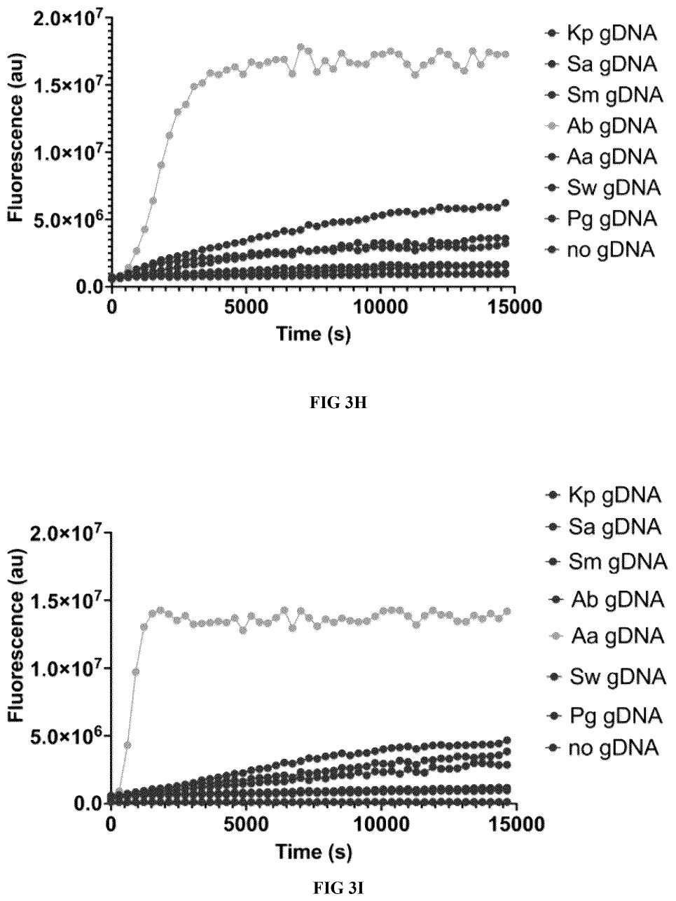

All seven crRNA-primer sets produced target-specific fluorescent signal over time, and at 35 minutes of detection, the fluorescent signals were greater than 19-fold change in the presence of target gDNA (1 pM; 600,000 copies/rxn) as compared to a negative control containing no target gDNA (FIG. 3A). These results confirmed that the computational pipeline produced crRNA-primer sets compatible with SHERLOCK detection. Cross-reactivity of the constructs was tested against the gDNA of the other bacterial targets (FIGS. 3B and 3C) using the “One-pot SHERLOCK Reaction (Cas13-RPA)s” as described above in Examples 1 and 2. Surprisingly, highly specific signals were achieved for all seven of the bacterial targets in this cross-reactivity assay. Remarkably, for six of the pathogens, A. actinomycetemcomitans, A. baumannii, K. pneumoniae, P. gingivalis, S. aureus, and S. mutans, the first crRNAs that were tested for each pathogen displayed highly specific signals and did not require the testing of a second crRNA. The only exception was S. wiggsiae: the first crRNA that was tested displayed a low, non-specific signal (Sw 1). Instead, the second S. wiggsiae crRNA that was tested (Sw 2) exhibited a strong, specific signal (FIG. 3C). At 35 minutes of detection, when in the presence of gDNA isolated from their intended target, all crRNA-primer sets produced a fluorescent signal greater than 10-fold change higher than off-target reactions, which can be seen in the individual plots (FIG. 3D-3I). These cross-specificity assays indicated that when tested against gDNA from off-target bacterial pathogens, all designed crRNA-primer sets produced a clear and discriminant signal only against their intended target.

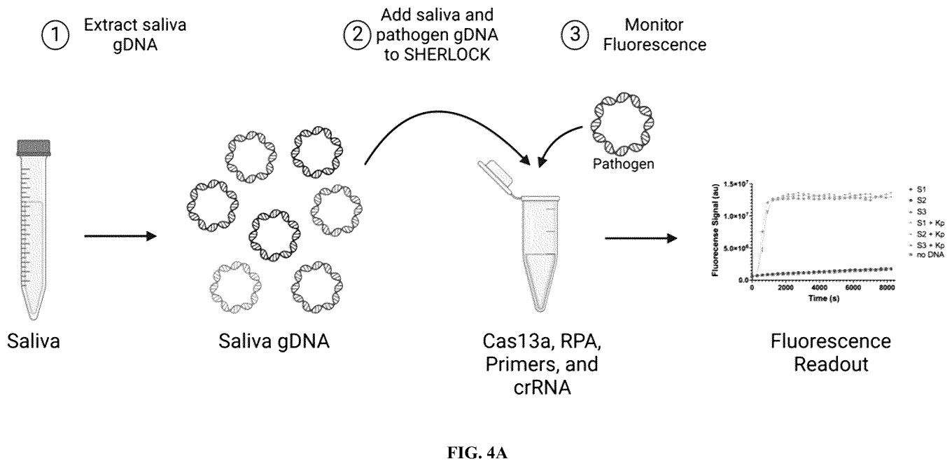

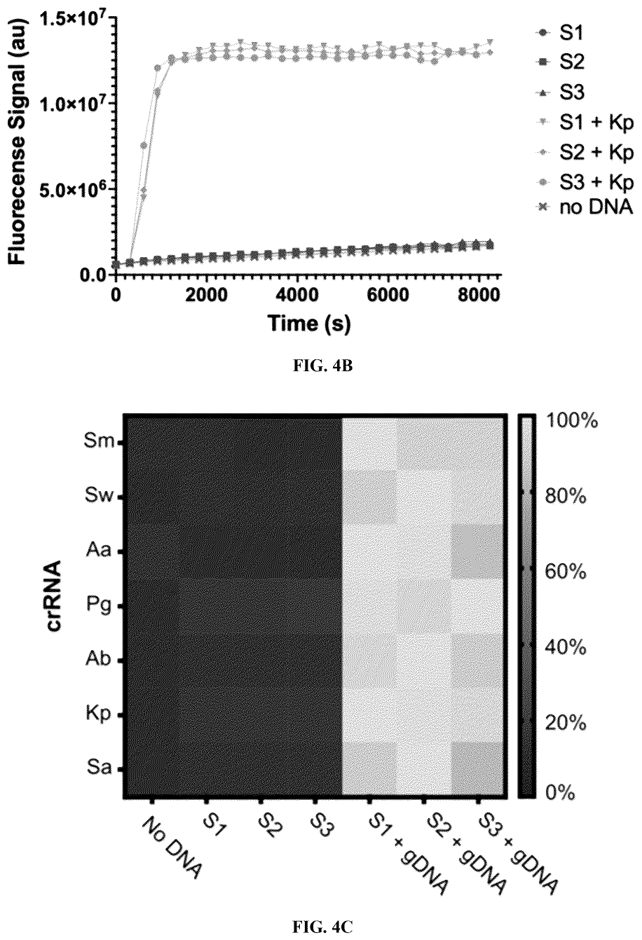

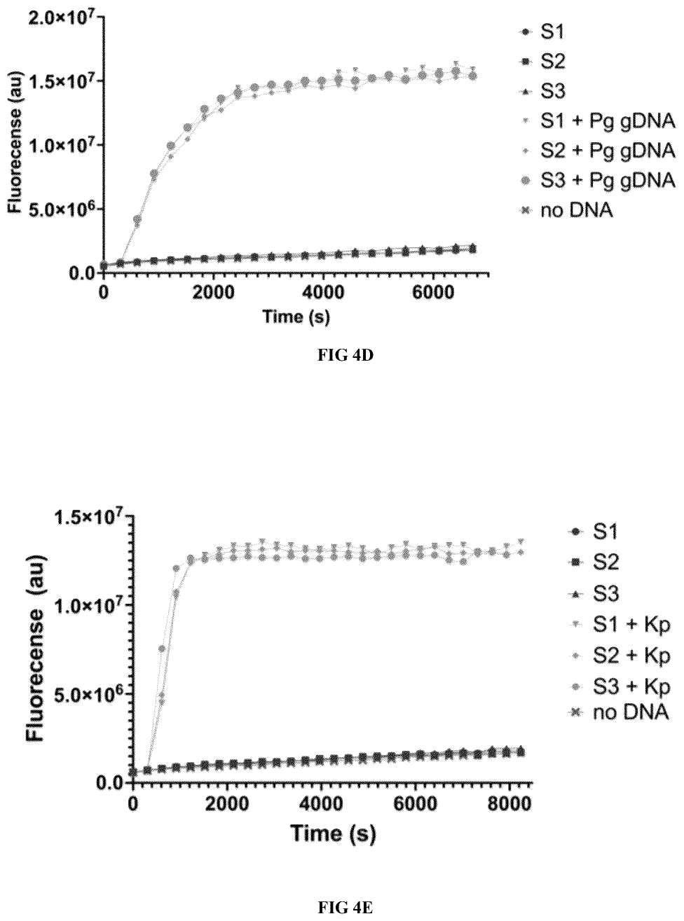

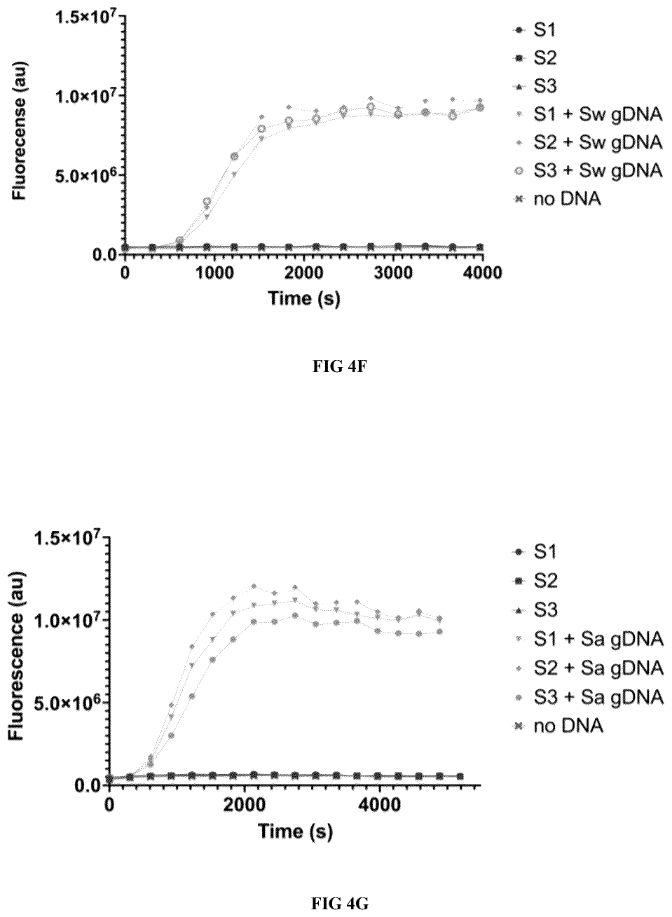

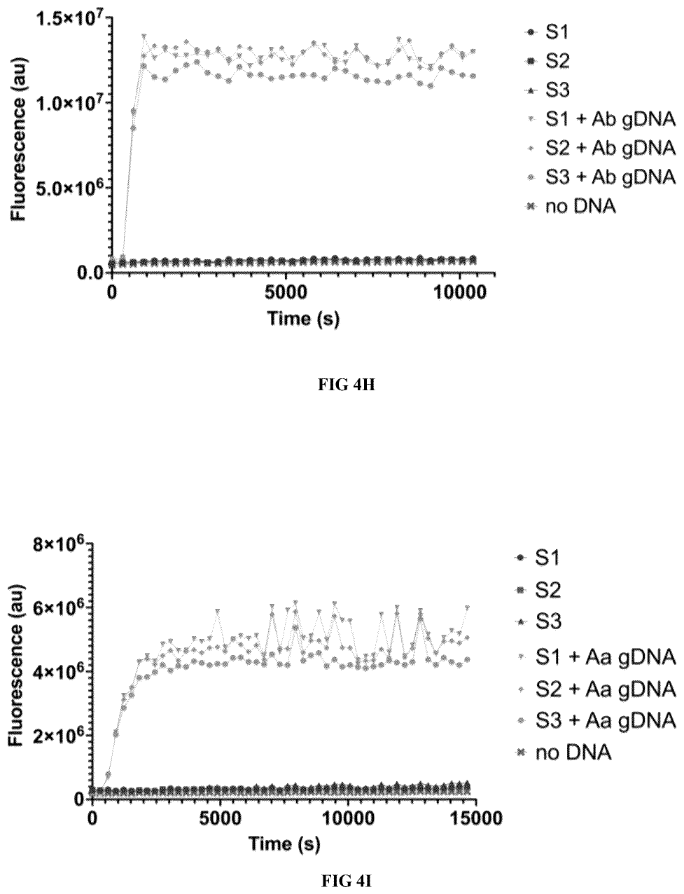

With no nonspecific signal detected in the cross-reactivity assay, the specificity of the crRNAs and primers against background gDNA isolated from saliva was further interrogated (FIGS. 4A-4I). Briefly, in order to test the specificity of the designed crRNAs against human DNA, DNA was isolated from three saliva samples using the MasterPure gram-positive DNA purification kit (Biosearch, #MC85200) according to the protocol referenced above. The fluorescence signal was surveyed at 35 minutes of detection produced by each crRNA-primer set when tested against 1) saliva gDNA (10 ng); 2) saliva gDNA (10 ng) spiked with target bacterial gDNA (1 pM, 600,000 copies/rxn); and 3) no saliva or target bacterial gDNA. 20 μL of each reaction was then transferred to individual wells on 384-well optical plate for fluorescence reading. In the presence of background saliva DNA, which contains commensal microbe and human gDNA, all seven of the crRNAs remained specific (FIG. 4C). Each crRNA-primer set displayed a greater than 12-fold change increase in fluorescent signal when in the presence of gDNA from their bacterial target as compared to saliva gDNA alone, which can be observed in the individual plots (FIGS. 4D-4I). Altogether, these results validated the specificity of the crRNA-primer sets in the presence of off-target oral bacterial and human gDNA.

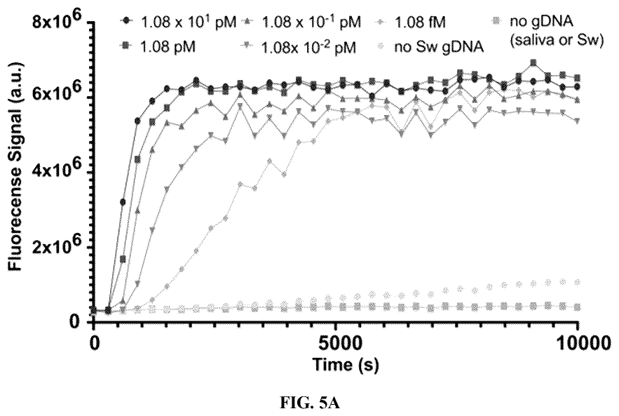

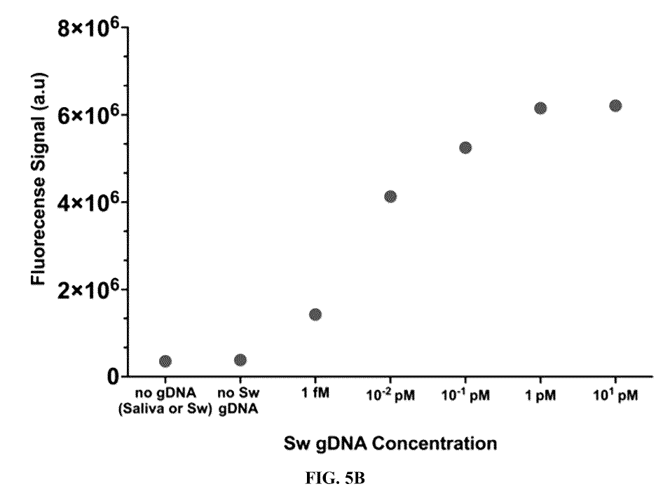

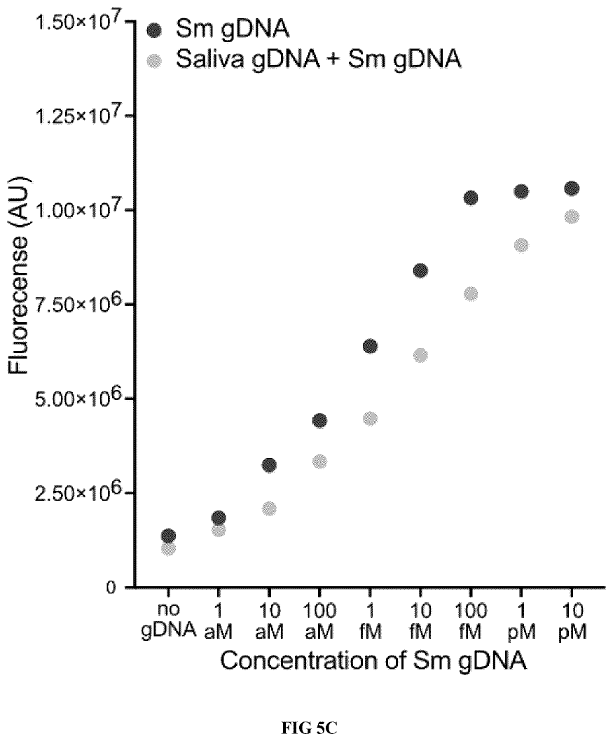

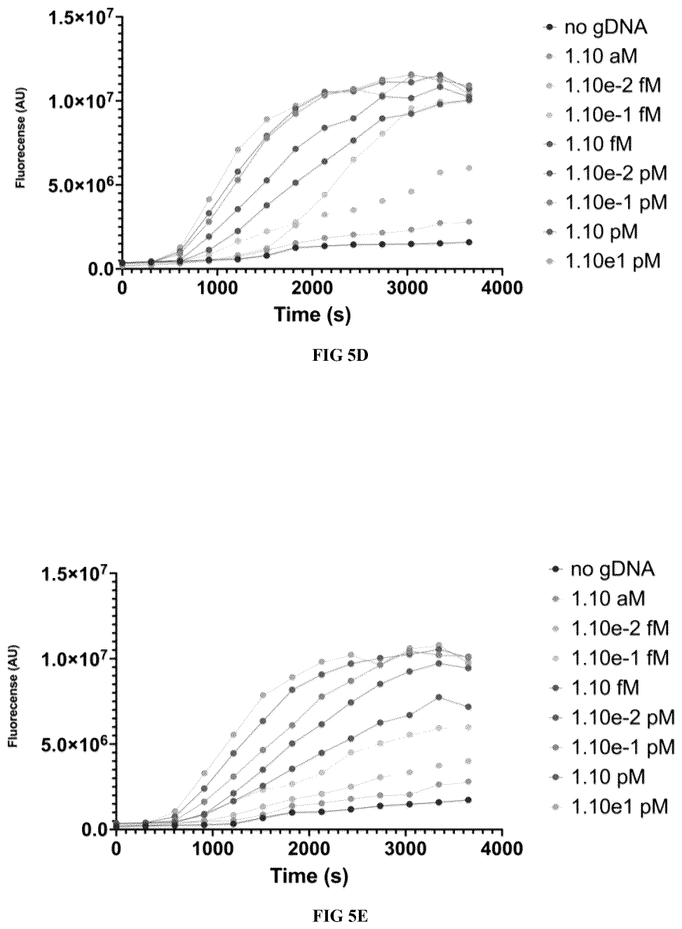

The sensitivity of designed crRNAs was additionally tested, a crRNA targeting S. wiggsiae, against a dilution series of target gDNA in the presence of background gDNA. Even in the presence of background gDNA, which included human gDNA and other bacterial gDNA, detection was achieved at the femtomolar range (˜600 copies/μL) (FIG. 5A). While at 35 minutes the femtomolar signal appears low relative to the other dilutions (FIG. 5B), all dilutions including at one femtomolar eventually reached saturation (FIG. 5A). These results indicate that the crRNAs are likely sensitive to even lower concentrations of target gDNA but may require more detection time. The addition of off-target saliva gDNA may also affect the sensitivity of SHERLOCK detection. To assess this, SHERLOCK detection was performed against a concentration gradient of Sm gDNA ranging from 1 attomolar (aM) (10 copy number/rxn) to 10 pM (6 million copy numbers/rxn) in two conditions: 1) with 10 ng of background saliva gDNA; and 2) without any background gDNA. Across the Sm gDNA dilutions, the saliva gDNA background dampened the fluorescent signal strength by an average of 20% at 35 minutes of detection (FIG. 5C). Despite this decrease in signal strength incurred by the addition of saliva gDNA, at the lowest concentration of Sm gDNA, 1 aM, a fluorescent signal 1.4 fold change greater was achieved with Sm gDNA than without Sm gDNA in a background of saliva gDNA (FIG. 5D-5E). The ability of this assay, therefore, to detect target sequences down to a concentration of 1 aM (10 copy number/rxn) was not hindered by the addition of a saliva gDNA background.

The addition of off-target saliva gDNA may also affect the sensitivity of SHERLOCK detection. To assess this, SHERLOCK detection was performed against a concentration gradient of Sm gDNA ranging from 1 aM (10 copy number/rxn) to 10 pM (6 million copy numbers/rxn) in two conditions: 1) with 10 ng of background saliva gDNA; and 2) without any background gDNA. Across the Sm gDNA dilutions, the saliva gDNA background dampened the fluorescent signal strength by an average of 20% at 35 minutes of detection (FIG. 5C). Despite this decrease in signal strength incurred by the addition of saliva gDNA, at the lowest concentration of Sm gDNA, 1 aM, a fluorescent signal 1.4 fold change greater was achieved with Sm gDNA than without Sm gDNA in a background of saliva gDNA (FIGS. 5D-5E). The ability of this assay, therefore, to detect target sequences down to a concentration of 1 aM (10 copy number/rxn) was not hindered by the addition of a saliva gDNA background.

Altogether, seven of eight of the tested crRNAs displayed highly specific signals both in the presence of gDNA from other pathogens and saliva (FIGS. 3A-3I and 4A-4I, Table 6). These data were remarkable and suggest that crRNAs targeting conserved species-specific genes rather than 16S rRNA genes have a higher propensity for specific detection. This method of targeting conserved species-specific genes rather than the 16S rRNA gene in bacteria avoids much of the trial-and-error testing associated with crRNAs.

### Example 4. Highly Specific Detection Directly from Saliva Samples

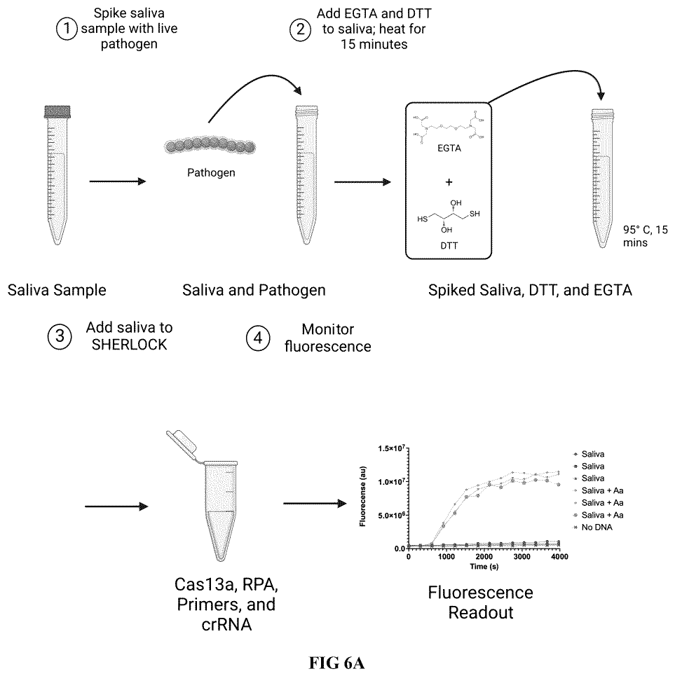

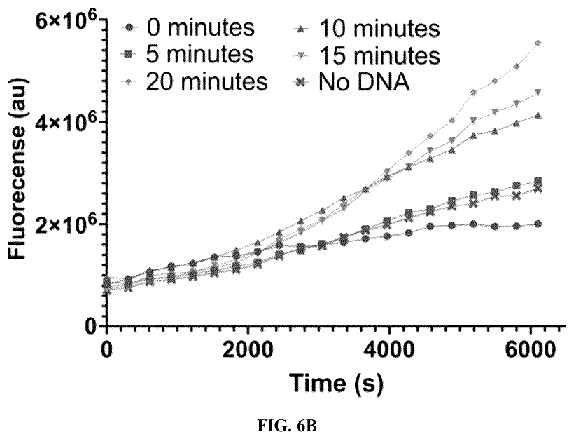

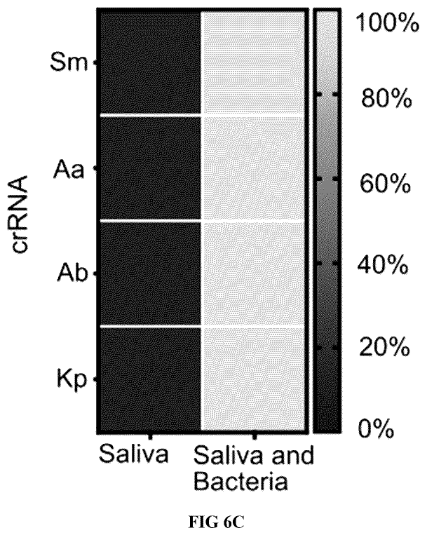

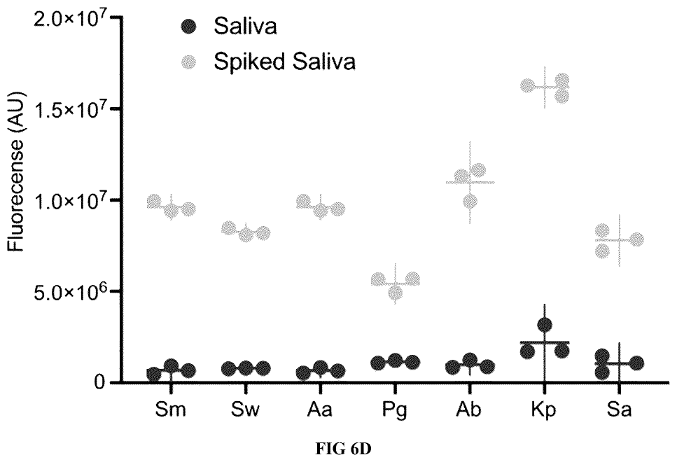

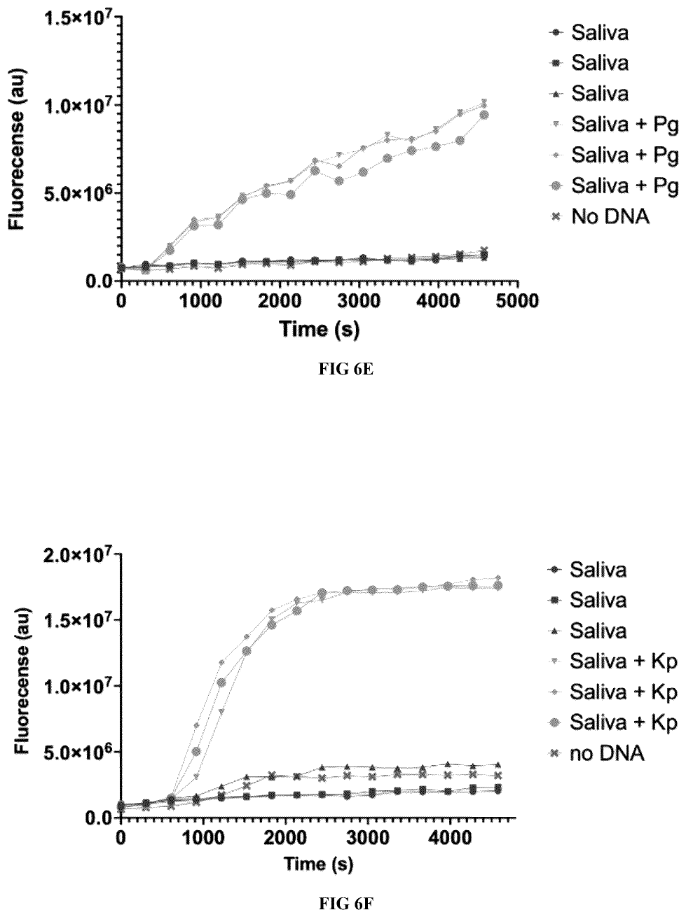

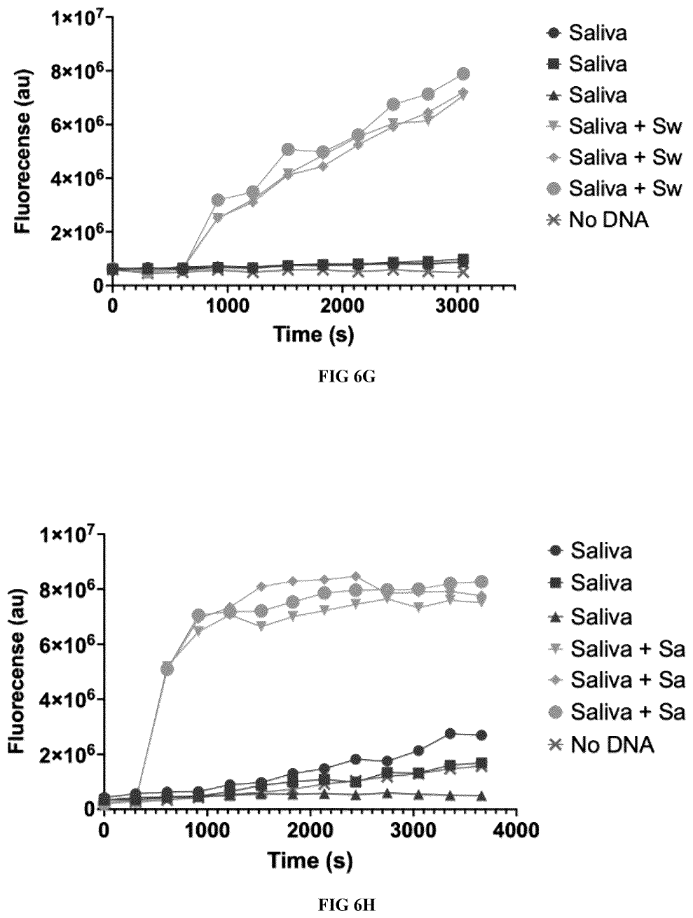

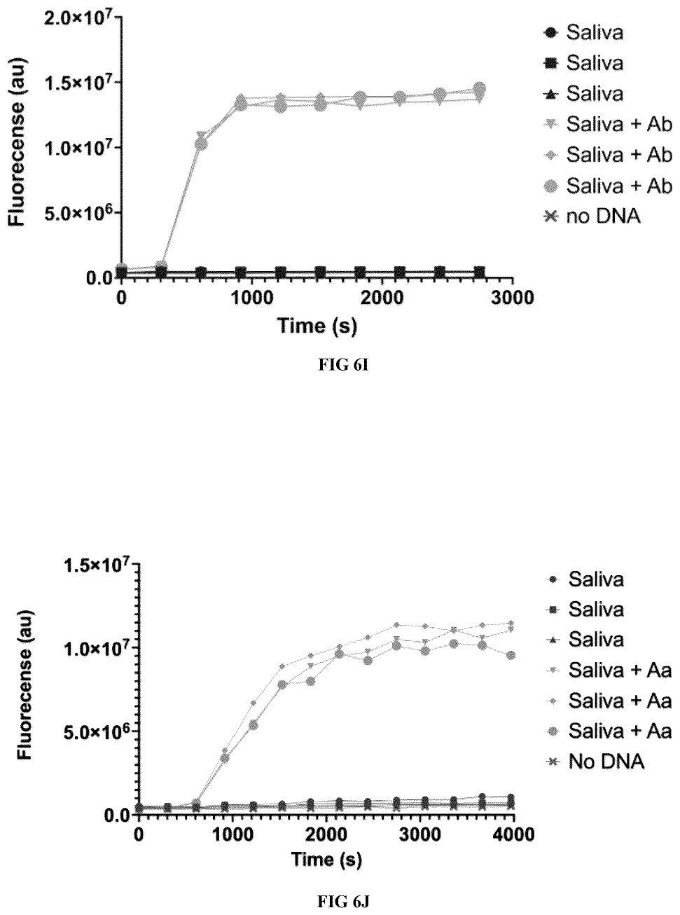

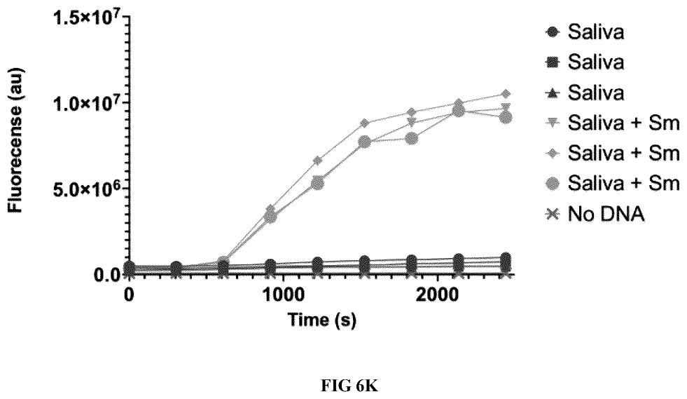

The above results demonstrated that the crRNAs designed to target species-specific genes of oral bacteria provided highly specific detection. Thus, the SHERLOCK assay was further translated for clinical use to directly test saliva samples. To achieve this, a previously described protocol was tailored to make SHERLOCK-EGTA+DTT capable of bacterial detection directly from saliva samples (FIG. 6A).

Briefly, in order to test the specificity of the designed crRNAs targeting the species-specific genes of A. actinomycetemcomitans (Aa), A. baumannii (Ab), K. pneumoniae (Kp), S. mutans (Sm), P. gingivalis (Pg), S. aureus (Sa), and S. wiggsiae (Sw) (Table 6) directly in saliva, two 1 mL samples of fresh saliva were collected. One of the saliva samples was then spiked with target bacteria from fresh liquid culture for a final concentration of 300 CFUs/reaction. The concentration of target bacteria was determined by taking OD600 readings and using previously published standard growth curves to determine CFUs/pL (Zhang 2015; Stahl 2015; Kim 2013; Castillo-Ruiz 2011). 20 μL of 500 mM EGTA and 20 μL of 1 mM DTT was added to both samples before being pulsed (i.e., vortexed) for 3 seconds. 10 μL of each sample was added to a strip tube and heated in a thermocycler at 95° C. for 15 minutes. 4 μL of each sample was added to separate aliquots of 20.6 μL of Cas mix using the “One-pot SHERLOCK Reaction (Cas13-RPAs)” described above in Examples 1 and 2. The crRNAs from Table 6 and primers from Table 7 were used in the assay. 20 μL of each of these reactions was then transferred to individual wells on 384-well optical plate for fluorescence reading (FIG. 6A). This modified SHERLOCK is herein referred to as SHERLOCK-EGTA+DTT.

In optimizing the direct-saliva testing protocol in tandem with testing the specificity of the crRNAs, two notable results were obtained. First, whether lysis of the bacterial targets was achievable by heating at 95° C. was assessed. It was determined that 15 minutes was the optimal minimum time to heat the samples (FIG. 6B). Second, highly specific detection was validated with the designed crRNAs in saliva for A. actinomycetemcomitans, A. baumannii, K. pneumoniae, S. mutans, P. gingivalis, S. aureus, and S. wiggsiae (FIGS. 6C-6K).

All seven crRNA-primer sets produced a fluorescent signal greater than 6-fold change higher when tested against unprocessed saliva spiked with live target bacterium (300 cfu/mL) compared to unprocessed saliva without the target bacteria (FIGS. 6D-6K). As observed in the isolated gDNA detection (FIG. 4C), the different crRNAs displayed varying levels of fluorescent signal at 35 minutes of detection (FIG. 60). However, this did not affect detection results. In no reaction did unprocessed saliva samples without spiked target bacteria produce a fluorescent signal at 35 minutes greater than 19% of samples with the target bacteria (FIGS. 6E-6K). These two observations indicated that the added reagents and additional heating step in SHERLOCK-EGTA+DTT effectively lysed bacterial cells and neutralized endogenous RNase activity and that the designed crRNA-primers sets remained effective when used for detection from unprocessed saliva samples. These results indicate that the treatment effectively inhibits the inherent RNAse activity present in saliva and that the detection is specific even in the presence of mucins, diverse proteins, and other cellular detritus.

Taken together, these results, achievable within an hour, demonstrate the clinical utility of the platform and open the door to future applications at routine dental appointments. Further, given the high efficacy of the designed species-specific crRNAs, this methodology could be applied at scale for the detection of almost any oral bacteria. In certain embodiments, one or more oral bacteria may be detected. For example, skilled artisans would understand that multiple oral bacteria may be detected using the SHERLOCK-EGTA+DTT system (Kellner 2019; Ackerman 2020).

### Example 5: Detection of Bacteria from Unprocessed Saliva Using SHERLOCK-EGTA+DTT

Next, the efficiency of SHERLOCK-EGTA+DTT detection of live bacteria in unprocessed saliva samples using EGTA and DTT (FIG. 6A) was compared to SHERLOCK detection of isolated gDNA directly. The two methodologies were compared using gram-negative Pg and gram-positive Sm.

Briefly, EGTA and DTT were added at a concentration of 500 mM and 1 mM, respectively, to a saliva sample collected from a healthy subject. The saliva sample was then heated at 95° C. for 15 minutes to facilitate bacterial lysis and inactivation of endogenous RNases. The saliva mixture, in lieu of target gDNA, was then used in a one-pot SHERLOCK-EGTA+DTT assay. The same methods described in Example 4 for the SHERLOCK-EGTA-DTT assay were used. In particular, the same “One-pot SHERLOCK Reaction (Cas13-RPA)” described above in Examples 1 and 2 and the crRNAs from Table 6 and primers from Table 7 were used in the assay.

To evaluate the efficacy of this method, the fluorescent signal produced by the Pg and Sm crRNA-primer sets were compared under two conditions: 1) One-pot SHERLOCK-EGTA+DTT with an unprocessed saliva sample spiked with live target bacteria; and 2) One-pot SHERLOCK with target gDNA. To equate spiked bacteria in the unprocessed saliva sample (condition 1) to isolated target gDNA (condition 2), the CFU/μL of live bacteria was estimated using previously established optical density (OD) to CFU measurements for both Pg and Sm (Tavares 2018; Kim 2013). Live bacteria was then added to unprocessed saliva samples (condition 1) at a CFU/μL estimated to be equivalent to the gDNA copy number in condition 2. Unprocessed saliva samples were collected from medically healthy subjects with no oral and periodontal disease or condition.

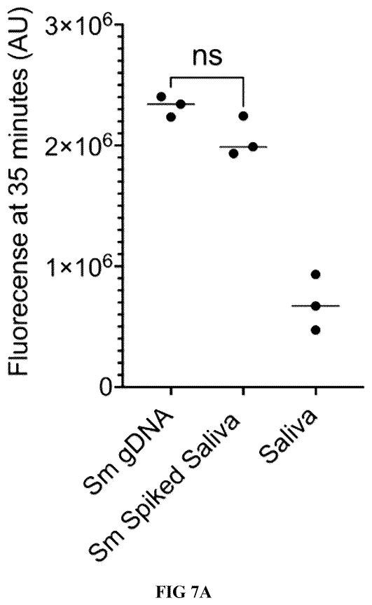

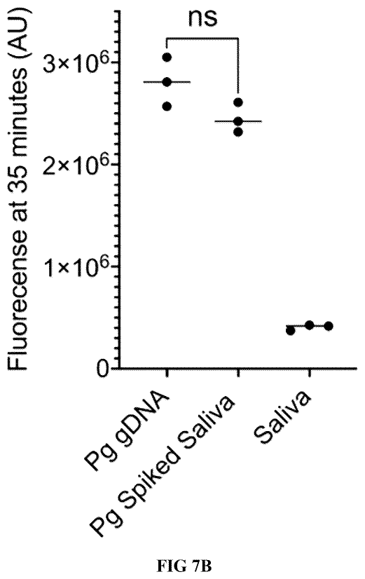

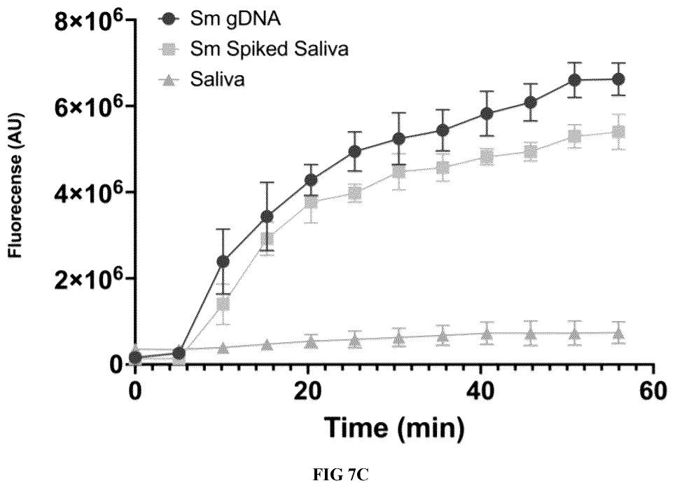

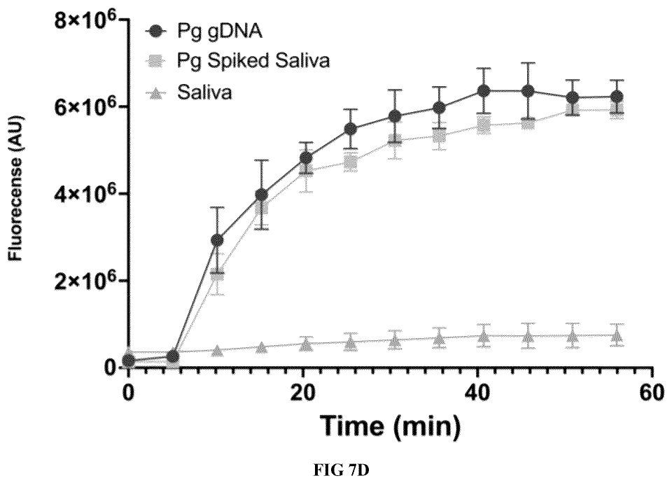

At 35 minutes of Sm or Pg detection, when equivalent amounts of cfu/mL and gDNA copy numbers were added to SHERLOCK-ETGA+DTT and SHERLOCK, respectively, no significant differences were observed between the fluorescent signals produced by the two methodologies (FIGS. 7A-7D). Therefore, in the case of Sm and Pg detection, the SHERLOCK-EGTA+DTT methodology is presumably as effective as SHERLOCK is at detecting target nucleic acid sequences from isolated gDNA.

### Example 6. Detection of Target Bacteria in Clinical Samples Aligns with 16S rRNA Sequence Profiling

To be effective in a clinical setting, detection needs to be similarly sensitive and accurate compared to established methods such as 16S rRNA sequencing. To this end, 30 saliva samples were collected from healthy subjects and 16S rRNA sequencing was performed on all 30 healthy saliva samples. 16S rRNA sequencing method uses broad range (V3-V4 16S) primer sets to amplify all bacterial species in the sample, followed by illumine sequencing to determine bacterial prevalence and abundance in the saliva sample. Due to this lengthy procedure, 16S rRNA sequencing method is expensive, lengthy, complex and require expertise in computational bioinformatics. In comparison, SHERLOCK-EGTA+DTT method avoids all these drawbacks.

Briefly, unstimulated saliva samples (1.5 mL) were collected from 30 adults (18 females, 12 males) with documented medical, dental and periodontal health as previously described (PMID: 33611834). The study received Forsyth Institutional Review Board approval, and all subjects provided written informed consent. Saliva samples were split into two aliquots: one for 16S rRNA sequencing and one for SHERLOCK-EGTA+DTT detection. gDNA was isolated from an aliquot of each saliva sample using the protocol described above. V3-V4 16S sequencing was then performed on all 30 samples (Zymogen). 16S rRNA relative abundances by 16S rRNA sequencing were calculated through an in-house Zymogen analysis, which, briefly, utilizes DADA2 (Callahan 2016) and a proprietary 16S rRNA database. For SHERLOCK-EGTA+DTT detection, the second aliquot of each saliva sample was diluted 1:4 with sterile PBS.

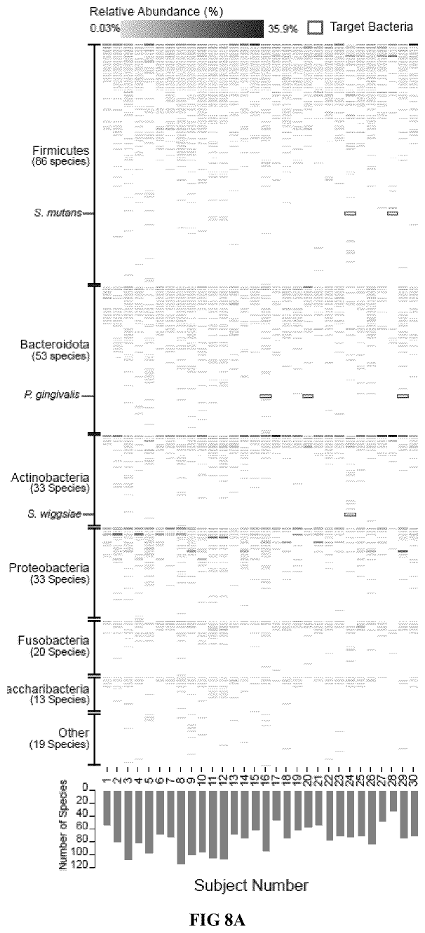

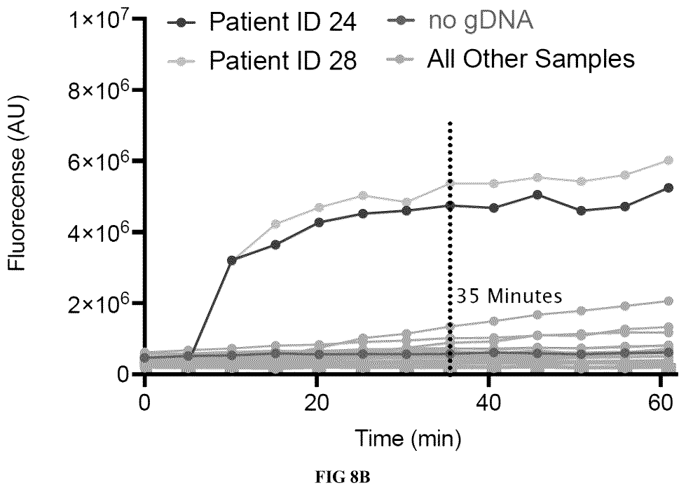

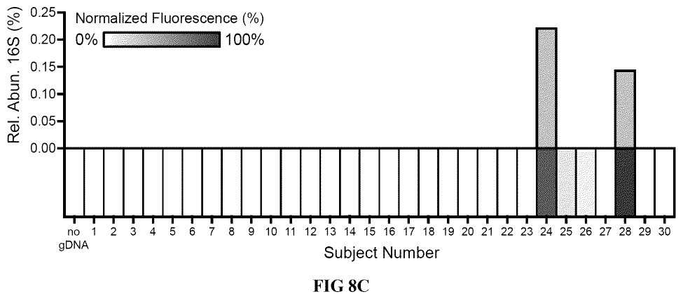

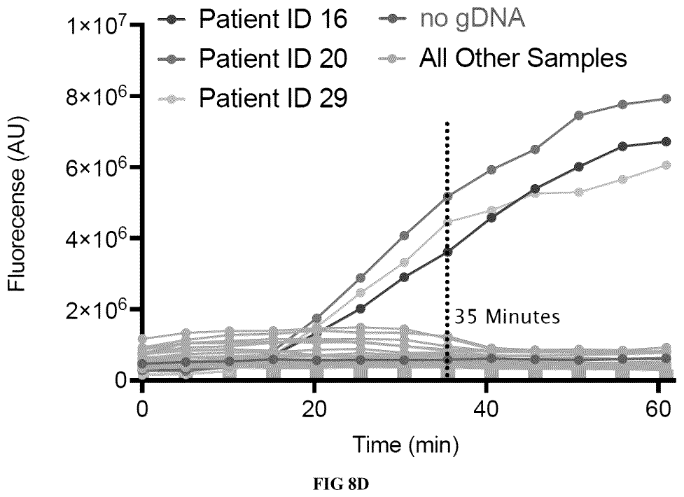

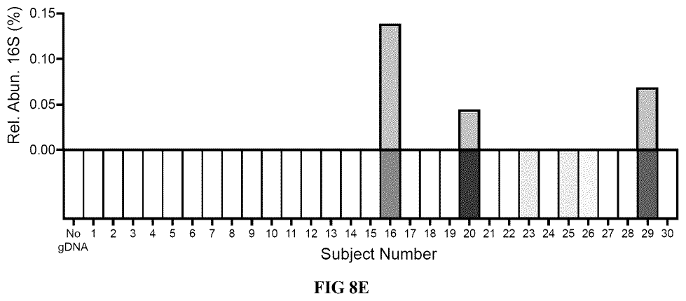

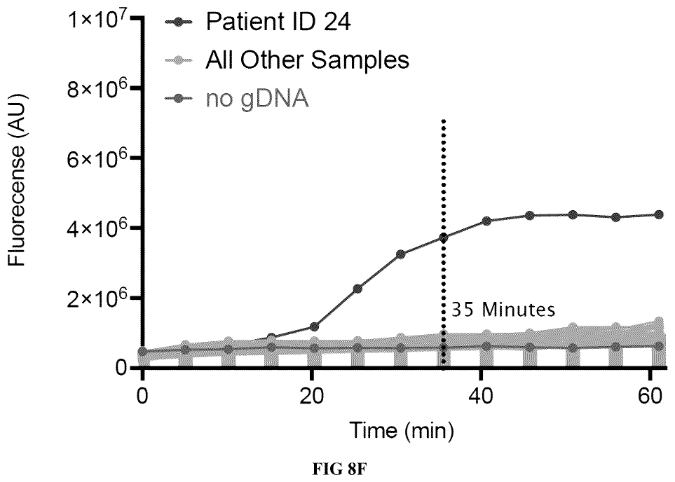

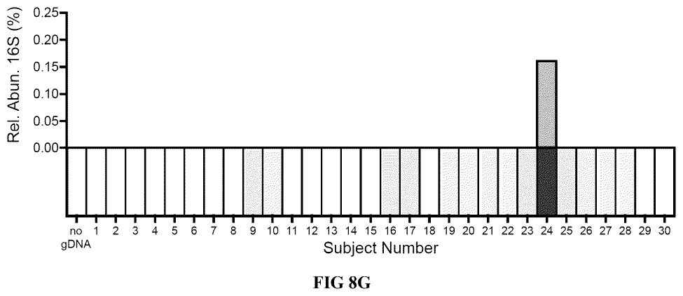

All samples were deidentified and numbered 1-30. Within the 30 saliva samples, 16S rRNA sequencing identified 257 different bacterial species (FIG. 8A). These saliva samples each contained between 34 and 115 bacterial species, a typical range found in other studies (Aas 2005). Of the seven candidate oral pathogens (A. actinomycetemcomitans, A. baumannii, K. pneumoniae, P. gingivalis, S. aureus, S. mutans, and S. wiggsiae), 16S rRNA sequencing identified two (Sample #24 and #28), three (Sample #16, 20, and 29), and one (Sample #24) samples positive for S. mutans (Sm), P. gingivalis (Pg), S. wiggsiae (Sw), respectively (FIG. 8A). None of the other four bacterial targets (A. actinomycetemcomitans (Aa), A. baumannii (Ab), K. pneumoniae (Kp), S. aureus (Sa)) were identified in any of the 30 samples by 16S rRNA sequencing. This was not surprising given that these bacteria are rarely found in healthy oral cavities (Eren 2014).

SHERLOCK-ETGA+DTT was performed on all thirty samples using the Sm, Pg, and Sw crRNA-primer sets set forth in Table 7. By Sm detection, samples 24 and 28 produced a signal greater than 8-fold change over a no gDNA control, while no other samples produced a signal greater than 1.8-fold change over the negative control (FIG. 8B). These results completely aligned with the 16S rRNA detection of Sm (FIG. 8C). Similarly, for Pg detection by SHERLOCK-EGTA+DTT, three fluorescent signals were observed that were over 6-fold change greater than the negative control while no other samples produced a signal greater than 1.7 (FIG. 80). The three samples that produced discernible signal, samples 16, 20, and 29, were the same samples that were identified to contain Pg by 16S rRNA sequencing (FIG. 8E). In Sw SHERLOCK-EGTA+DTT detection, only one discernible signal was observed: the signal produced by sample 24 was 6.5-fold change greater than the negative control, while all other samples were no more than 1.5-fold change greater (FIG. 8F). As observed in Sm and Pg detection, only the sample identified to contain Sw by 16S rRNA sequencing produced a discernable signal by SHERLOCK-EGTA+DTT detection FIG. 8G). Altogether, the SHERLOCK-EGTA+DTT detection results accurately recapitulated the 16S rRNA sequencing results for Sm, Pg, and Sw presence (100% agreement between the two methods).

In sum, a computational pipeline capable of generating species-specific crRNAs and primer pairs for SHERLOCK was designed and demonstrated highly specific and sensitive detection for seven established oral pathogens. Further, SHERLOCK-EGTA+DTT was tailored for the detection of oral bacteria directly from unprocessed saliva samples and validated the detection on clinical samples. The results suggest that the crRNA-primer sets paired with SHERLOCK-EGTA+DTT are on par with canonical detection techniques such as 16S rRNA sequencing. Therefore, SHERLOCK-EGTA+DTT detection and sensitivity is equal to 16S rRNA sequencing method, but much more simpler, faster and cost effect way to detect these bacteria.

Although saliva contains numerous compounds that could interfere with SHERLOCK detection, such as endogenous RNases, off-target nucleic acid sequences, and salivary mucins, no interference was observed in the results using the SHERLOCK-EGTA+DTT methodology. This modification is a step forward for SHERLOCK detection from unprocessed saliva samples that could provide increased flexibility to consumers and medical personnel.

As more oral microorganisms and their molecular underpinnings are implicated with disease, it is expected that the need for a reliable, rapid detection system will continue to grow. With this expectation in mind, the multiplexing of SHERLOCK detection for oral microorganisms will likely be a necessary future advancement. Cas13-based detection methods have been multiplexed in other scenarios (Kellner 2019; Ackerman 2020). The CARMEN system, for instance, allows for the simultaneous detection and subtyping of 100+ human viruses (Ackerman 2020). Implementation of such multiplexing with constructs from the computational pipeline described herein could allow for the simultaneous detection of many oral microorganisms. It is conceivable that with multiplexed detection, the comprehensive profiling of an individual's oral microbiome from a single saliva sample could be achieved.

SHERLOCK has also been proposed as a healthcare tool for under-resourced areas. SHERLOCK reagents, including crRNAs and primers, can be lyophilized and employed on a laminar flow strip for an estimated cost of $0.60 per single-plex assay. Translating the constructs from this study into a similar application may allow for detection beyond traditional point-of-care dental settings and into underserved areas. While detection may be the first step towards treatment, precise treatments designed to eliminate specific species, and even strains, of oral pathogens are gaining momentum. Coupled with targeted treatment, a rapid detection system such as the one proposed here is an important step forward toward individualized and precise oral care.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### INCORPORATION BY REFERENCE

All references mentioned herein are hereby incorporated by reference in their entirety as if each individual reference was specifically and individually indicated to be incorporated by reference.

### REFERENCES

Aas, J. A., et al. J. Clin. Microbiol. 43, 5721-5732 (2005)
Ackerman, C. M., et al. Nature 582, 277-282 (2020)
Beghini, F. et al., eLife 10, e65088 (2021)
Blanco-Miguez, A., et al. Extending and improving metagenomic taxonomic profiling with uncharacterized species with MetaPhIAn 4. 2022.08.22.504593 Preprint at https://doi.org/10.1101/2022.08.22.504593 (2022)
Callahan, B. J., et al. Nat. Methods 13, 581-583 (2016)
Castillo-Ruiz, et al., Appl. Environ. Microbiol. 77:3157-3159 (2011)
Cock, P. J. A., et al. Bioinformatics 25, 1422-1423 (2009)
Eren, A. M., et al. Proc. Natl. Acad. Sci. 111, E2875-E2884 (2014)
Escapa, I. F., et al., mSystems 3, e00187-18
Gootenberg, J. S., et al. Science 356, 438-442 (2017)
Katoh, et. al., Nucleic Acids Res. 30, 3059-3066 (2002)
Kellner, M. J., et al. Nat. Protoc. 14, 2986-3012 (2019)
Kim, S., et al. Restor. Dent. Endod. 38, 65-72 (2013)
Lorenz, R., et al. ViennaRNA Package 2.0. Algorithms Mol. Biol. 6, 26 (2011)
Stahl, et al., PLoS ONE 10, e0138360 (2015)
Tavares, L. J., et al. J. Periodontal Implant Sci. 48, 12 (2018)
Untergasser, A., et al. Nucleic Acids Res. 40, e115 (2012)
Zhang, et al., J. Pure Appl. Microbiol. 9(1):299-305 (2015)

1. A CRISPR-RNA (crRNA) comprising a spacer that binds a target nucleic acid sequence in a species-specific gene in an oral bacterium.

2. (canceled)

3. The crRNA of claim 1, wherein the oral bacterium is from a species selected from the group consisting of Aggregatibacter actinomycetemcomitans, Acinetobacter baumannii, Klebsiella pneumoniae, Porphyromonas gingivalis, Staphylococcus aureus, Streptococcus mutans, and Scardovia wiggsiae.

4. The crRNA of claim 3, wherein the species-specific gene comprises a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus).

5. The crRNA of claim 4, wherein the spacer comprises a sequence that is the same as, or differs no more than 1, 2, 3, 4, or 5 nucleotides from, a sequence selected from the group consisting of the spacer RNA sequences set forth in SEQ ID NOs:83-89, and 122.

6-25. (canceled)

26. A CRISPR-nuclease detection system comprising
(i) an RNA-guided nuclease; and
(ii) a first crRNA comprising a spacer that binds a target nucleic acid sequence in a species-specific gene in an oral bacterium.

27. (canceled)

28. The CRISPR-nuclease detection system of claim 26, wherein the oral bacterium is from a species selected from the group consisting of Aggregatibacter actinomycetemcomitans, Acinetobacter baumannii, Klebsiella pneumoniae, Porphyromonas gingivalis, Staphylococcus aureus, Streptococcus mutans, and Scardovia wiggsiae.

29. The CRISPR-nuclease detection system of claim 28, wherein the species-specific gene comprises a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus).

30. The CRISPR-nuclease detection system of claim 29, wherein the spacer comprises a sequence that is the same as, or differs no more than 1, 2, 3, 4, or 5 nucleotides from, a sequence selected from the group consisting of the spacer RNA sequences set forth in SEQ ID NOs:83-89, and 122.

31-37. (canceled)

38. The CRISPR-nuclease detection system of claim 30, wherein the RNA-guided nuclease is a Cas13 protein.

39. The CRISPR-nuclease detection system of claim 38, wherein the Cas13 protein is a Cas13a protein.

40-41. (canceled)

42. The CRISPR-nuclease detection system of claim 39, further comprising one or more components selected from the group consisting of a forward primer, a reverse primer, a reporter, replication protein A (RPA), RNAse inhibitor, ribonucleoside tri-phosphate (rNTP) mix, and T7 RNA Polymerase.

43-46. (canceled)

47. The CRISPR-nuclease detection system of claim 42, wherein the reporter comprises a molecule that releases a signal upon cleavage of the reporter by the RNA-guided nuclease.

48. A method of detecting the presence of one or more oral bacteria in a biological sample, comprising
(a) contacting the biological sample with a CRISPR-nuclease detection comprising:
(i) an RNA-guided nuclease; and
(ii) a first crRNA comprising a spacer that binds a target nucleic acid sequence in a species-specific gene in the oral bacterium; and
(b) detecting the presence of the one or more oral bacteria in the biological sample.

49-69. (canceled)

70. The method of claim 48, wherein the oral bacterium is from a species selected from the group consisting of Aggregatibacter actinomycetemcomitans, Acinetobacter baumannii, Klebsiella pneumoniae, Porphyromonas gingivalis, Staphylococcus aureus, Streptococcus mutans, and Scardovia wiggsiae.

71. The method of claim 70, wherein the species-specific gene comprises a sequence selected from the group consisting of SEQ ID NO:113 (S. mutans), SEQ ID NO:114 (S. wiggsiae), SEQ ID NO:115 (A. actinomycetemcomitans), SEQ ID NO:116 (P. gingivalis), SEQ ID NO:117 (A. baumannii), SEQ ID NO:118 (K. pneumoniae), and SEQ ID NO:119 (S. aureus).

72. The method of claim 71, wherein the spacer comprises a sequence that is the same as, or differs no more than 1, 2, 3, 4, or 5 nucleotides from, a sequence selected from the group consisting of the spacer RNA sequences set forth in SEQ ID NOs:83-89, and 122.

73. The method of claim 72, further comprising one or more components selected from the group consisting of a forward primer, a reverse primer, a reporter, replication protein A (RPA), RNAse inhibitor, ribonucleoside tri-phosphate (rNTP) mix, and T7 RNA Polymerase.

74. The method of claim 73, wherein the reporter comprises a molecule that releases a signal upon cleavage of the reporter by the RNA-guided nuclease.

75. The method of claim 74 wherein the biological sample is saliva.

76. The method of claim 75, wherein ethylene glycol-bis(β-aminoethyl ether)-N,N,N′,N′-tetraacetic acid (EGTA) and dithiothreitol (DTT) are added to the biological sample prior to step (a) of contacting the biological sample with the CRISPR-nuclease detection system.
